(19) United States
(12) Patent Application Publication
(10) Pub. No.: US 2022/0164628 A1
(43) Pub. Date: May 26, 2022

(54) SYSTEM AND METHOD FOR MODELING THE STRUCTURE AND FUNCTION OF BIOLOGICAL SYSTEMS

(71) Applicant: University of Southern California, Los Angeles, CA (US)

(72) Inventor: Joel Hahn, Los Angeles, CA (US)

(21) Appl. No.: 17/500,312

(22) Filed: Oct. 13, 2021

Related U.S. Application Data
(63) Continuation of application No. 15/706,564, filed on Sep. 15, 2017, now abandoned.
(60) Provisional application No. 62/396,001, filed on Sep. 16, 2016.

(51) Int. Cl. G06N 3/04; G06F 16/25; G16B 5/00; G16B 50/00; G16B 50/30; G16B 50/20

(52) U.S. Cl. CPC G06N 3/04; G06F 16/258; G16B 5/00; G06N 5/022; G16B 50/30; G16B 50/20; G16B 50/00

(57) ABSTRACT

Described herein are systems and methods for modeling the structure and function of the nervous system including the central nervous system and the peripheral nervous system. The system and methods provide novel tools for systematizing the construction of connectomes.

### CROSS-REFERENCE TO RELATED APPLICATION

This application is a continuation of U.S. patent application Ser. No. 15/706,564, filed Sep. 15, 2017, which claims priority under 35 U.S.C. § 119(e) to U.S. Provisional Application No. 62/396,001, filed Sep. 16, 2016, the content of each of which is incorporated herein by reference in its entirety.

### BACKGROUND

Throughout this disclosure, various technical and patent publications are referenced to more fully describe the state of the art to which this invention pertains, the full bibliographic citations for some of the publications may be found at the end of the specification, immediately preceding the claims. All publications noted in the present specification are incorporated by reference, in their entirety, into this application.

The nervous system is comprised of two major divisions: the Central Nervous System (CNS) and the Peripheral Nervous System (PNS). The CNS is comprised of the brain and spinal cord. The PNS is comprised of the nerves and ganglia that control the muscles and glands of the body. There is a need to identify and analyze networks between different regions, tissue, cell types, hubs, neurons, and/or circuits of the CNS and PNS to better understand their relationships and function from a network perspective. The present disclosure satisfies this need and provides related advantages as well.

### SUMMARY OF THE DISCLOSURE

This disclosure provides a system and method for modeling connections, structure, and/or function of biological systems, such as the nervous system including the CNS and/or PNS. In one aspect, the method comprises generating, with a processor, a plurality of connections corresponding to nervous system data, e.g., the CNS and/or PNS data, wherein each of the plurality of connections comprises an origin, a termination, and a degree of connection. The method also includes storing the plurality of connections in a first memory location, receiving a nervous system atlas, and storing the nervous system atlas in a second memory location. The method also includes matching, with a processor on a connection-by-connection basis, each origin and each termination of the plurality of connections to a corresponding position of the nervous system atlas to produce an annotated connection matrix. The method further includes converting the annotated connection matrix to one or more modules, wherein the one or modules comprise an aggregated ranking of connections exceeding a threshold.

In one aspect, the method further comprises deriving the data from CNS and/or PNS connection information. In another aspect, the method further comprises deriving the data from nervous system gene expression data, proteomic data, or markers of neural activity. In yet another aspect, the nervous system gene expression data comprises expressions of a neurotransmitter, a neurotransmitter receptor, or a nervous system cellular marker. In still yet another aspect, the method further comprises presenting the one or more modules as two-dimensional models. In yet another aspect, the method further comprises projecting the one or more modules onto an atlas of the nervous system (e.g. an atlas of the rat brain). In another aspect, the ranking of connections comprises one or more of a node degree, node strength, node betweenness and node closeness.

In a yet further embodiment, a method for modeling connections, structure, and/or function of the nervous system including the CNS and/or PNS comprises generating, with a processor, a matrix by use of a nervous system atlas. The method also includes storing the matrix in a first memory location, and receiving data of cerebral nuclei to generate a plurality of connections corresponding to the data of cerebral nuclei, where each of the plurality of connections comprises an origin, a termination, and a degree of connection The method also includes storing the plurality of connections in a second memory location, matching each origin and each termination of the plurality of connections to a corresponding position of the matrix to produce an annotated connection matrix, and converting the annotated connection matrix to one or more modules, where the one or modules comprise an aggregated ranking of connections exceeding a threshold.

The foregoing is a summary of the disclosure and thus by necessity contains simplifications, generalizations, and omissions of detail. Consequently, those skilled in the art will appreciate that the summary is illustrative only and is not intended to be in any way limiting. Other aspects, features, and advantages of the devices and/or processes described herein, as defined by the claims, will become apparent in the detailed description set forth herein and taken in conjunction with the accompanying drawings.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1A-1I show an Axiome template that facilitates entry, collation, and comparison of neural connection data in accordance with an illustrative embodiment. FIG. 1A shows a partial view of the Axiome template. FIG. 1B shows a partial view of the Axiome template. FIG. 1C shows a partial view of the Axiome template. FIG. 1D shows a partial view of the Axiome template. FIG. 1E shows a partial view of the Axiome template. FIG. 1F shows a partial view of the Axiome template. FIG. 1G shows a partial view of the Axiome template. FIG. 1H shows a partial view of the Axiome template. FIG. 1I shows a partial view of the Axiome template.

FIG. 2 shows another Axiome template that facilitates entry, collation, and comparison of neural expression data in accordance with an illustrative embodiment.

FIGS. 3A-3H show a user interface that generates real-time analysis and feedback by use of the Axiome template of FIGS. 1A-1I in accordance with an illustrative embodiment. FIG. 3A shows a partial view of the user interface. FIG. 3B shows a partial view of the user interface. FIG. 3C shows a partial view of the user interface. FIG. 3D shows a partial view of the user interface. FIG. 3E shows a partial view of the user interface. FIG. 3F shows a partial view of the user interface. FIG. 3G shows a partial view of the user interface. FIG. 3H shows a partial view of the user interface.

FIGS. 4A-4AB show a user interface that generates real-time analysis and feedback by use of the Axiome template of FIG. 2 in accordance with an illustrative embodiment. FIG. 4A shows a partial view of the user interface. FIG. 4B shows a partial view of the user interface. FIG. 4C shows a partial view of the user interface. FIG. 4D shows a partial view of the user interface. FIG. 4E shows a partial view of the user interface. FIG. 4F shows a partial view of the user interface. FIG. 4G shows a partial view of the user interface. FIG. 4H shows a partial view of the user interface. FIG. 4I shows a partial view of the user interface. FIG. 4J shows a partial view of the user interface. FIG. 4K shows a partial view of the user interface. FIG. 4L shows a partial view of the user interface. FIG. 4M shows a partial view of the user interface. FIG. 4N shows a partial view of the user interface. FIG. 4O shows a partial view of the user interface. FIG. 4P shows a partial view of the user interface. FIG. 4Q shows a partial view of the user interface. FIG. 4R shows a partial view of the user interface. FIG. 4S shows a partial view of the user interface. FIG. 4T shows a partial view of the user interface. FIG. 4U shows a partial view of the user interface. FIG. 4V shows a partial view of the user interface. FIG. 4W shows a partial view of the user interface. FIG. 4X shows a partial view of the user interface. FIG. 4Y shows a partial view of the user interface. FIG. 4Z shows a partial view of the user interface. FIG. 4AA shows a partial view of the user interface. FIG. 4AB shows a partial view of the user interface.

FIGS. 5A-5J show a comparison template that facilitates comparison between two exemplary datasets generated by use of the Axiome template of FIG. 2 in accordance with an illustrative embodiment. FIG. 5A shows a partial view of the comparison template. FIG. 5B shows a partial view of the comparison template. FIG. 5C shows a partial view of the comparison template. FIG. 5D shows a partial view of the comparison template. FIG. 5E shows a partial view of the comparison template. FIG. 5F shows a partial view of the comparison template. FIG. 5G shows a partial view of the comparison template. FIG. 5H shows a partial view of the comparison template. FIG. 5I shows a partial view of the comparison template. FIG. 5J shows a partial view of the comparison template.

FIG. 6 shows rat cerebral nuclei association connectome. Directed synaptic macroconnection matrix with gray matter region sequence in the topographically ordered nomenclature hierarchy is provided in Table 4. Grey scale of connection weights and properties is at the bottom. Abbreviations: AAA, anterior amygdalar area; ACB, accumbens nucleus; BA, bed nucleus of accessory olfactory tract; BAC, bed nucleus of anterior commissure; BSTal, anterolateral area of bed nuclei of terminal stria; BSTam, anteromedial area of bed nuclei of terminal stria; BSTd, dorsal nucleus of bed nuclei of terminal stria; BSTdm, dorsomedial nucleus of bed nuclei of terminal stria; BSTfu, fusiform nucleus of bed nuclei of terminal stria; BSTif, interfascicular nucleus of bed nuclei of terminal stria; BSTju, juxtacapsular nucleus of bed nuclei of terminal stria; BSTmg, magnocellular nucleus of bed nuclei of terminal stria; BSTov, oval nucleus of bed nuclei of terminal stria; BSTpr, principal nucleus of bed nuclei of terminal stria; BSTrh, rhomboid nucleus of bed nuclei of terminal stria; BSTse, strial extension of bed nuclei of terminal stria; BSTtr, transverse nucleus of bed nuclei of terminal stria; BSTv, ventral nucleus of bed nuclei of terminal stria; CEAc, capsular part of central amygdalar nucleus; CEA1, lateral part of central amygdalar nucleus; CEAm, medial part of central amygdalar nucleus; CP, caudoputamen; FS, striatal fundus; GP1, lateral globus pallidus; GPm, medial globus pallidus; IA, intercalated amygdalar nuclei; LSc.d, dorsal zone of caudal part of lateral septal nucleus; LSc.v, ventral zone of caudal part of lateral septal nucleus; LSr.dl, dorsolateral zone of rostral part of lateral septal nucleus; LSr.m.d, dorsal region of medial zone of rostral part of lateral septal nucleus; LSr.m.v, ventral region of medial zone of rostral part of lateral septal nucleus; LSr.vl, ventrolateral zone of rostral part of lateral septal nucleus; LSv, ventral part of lateral septal nucleus; MA, magnocellular nucleus; MEAad, anterodorsal part of medial amygdalar nucleus; MEAav, anteroventral part of medial amygdalar nucleus; MEApd, posterodorsal part of medial amygdalar nucleus; MEApv, posteroventral part of medial amygdalar nucleus; MS, medial septal nucleus; NDB, diagonal band nucleus; OT, olfactory tubercle; SF, septofimbrial nucleus; SH, septohippocampal nucleus; SI, innominate substance; TRS, triangular septal nucleus.

FIGS. 7A and 7B show in degree/out degree (FIG. 7A) and in strength/out strength (FIG. 7B) for all regions of the single-hemisphere rat cerebral nuclei association macroconnection network. Regions are ranked by total degree, in descending order. Greyscale indicates the asymmetry in in/out degree and in/out strength, respectively, computed as (in degree−out degree)/(in degree+out degree) and (in strength−out strength)/(in strength+out strength). A value of −1 (light grey) indicates strong prevalence of out degree/strength (the area is a “sender”) and a value of +1 (dark grey) indicates a strong prevalence of in degree/strength (the area is a “receiver”). For abbreviations see the description of FIG. 6.

FIGS. 8A-8E show stability of module partitions under variation of spatial resolution parameter γ. FIGS. 8A-8D show four stable Q:14 module partitions encountered in the range γ=[0.5 1.5], with regions within modules arranged by their total node strength. FIG. 8A shows one of the four stable Q:14 module partitions. FIG. 8B shows the second of the four stable Q:14 module partitions. FIG. 8C shows the third of the four stable Q:14 module partitions. FIG. 8D shows the fourth of the four stable Q:14 module partitions. FIG. 8E plots the number of modules encountered at each level of γ. All four partitions shown here remain stable across their respective ranges of γ, as verified by computing the variation of information across partitions. Arrows between FIGS. 8A-8D indicate modules that remain unchanged across different solutions. Note that M4 is robustly detected at all levels of γ. An unstable solution encountered around γ=1.2 is excluded from the analysis. The four-module solution (FIG. 8A) is adopted in the remainder of the study. Region abbreviations along the axes for A are provided in the same sequence as in FIG. 9 and are defined in the description of FIG. 6. The scale refers to the seven weight categories (1, very weak; 2, weak; 3, weak to moderate; 4, moderate; 5, moderate to strong; 6, strong; 7, very strong) of existing connections, and 0 corresponds to connections that are shown to be absent or for which there are no data.

FIG. 9 shows weighted connection matrix (log₁₀ scale) for 45 (single-hemisphere) areas. Ordering is as for the four-module matrix in FIG. 8A, with regions within modules arranged by total node strength. For abbreviations see the description for FIG. 6.

FIGS. 10A and 10B show layout diagrams of connection patterns in a single hemisphere. (FIG. 10A) Nodes (regions) and edges (connections) are projected onto two dimensions, using a Fruchterman-Reingold energy minimization layout algorithm. Nodes are: M1: CP, GPI, PGm, and FS; M2: OT, BSTju, SI, BSTam, CEAc, BSTal, BSTrh, CEAm, BSTfu, ACB, BSTov, CEAI, BSTmg, BSTv, and BSTdm; M3: BSTse, IA, MEAav, AAA, BSTtr, MEAad, MEApv, BA, BSTd, BSTif, MA, MEApd, BAC, BSTpr, LSr.vl, and LSv; M4: LSR.dl, LSr.m.v, TRS, LSc.v, LSr.m.d, SF, MS, LSc.d, NDB, and SH. To simplify the plot, connections are drawn without reference to directionality (gray color level and thickness of line proportional to log 10 of connection weight). (FIG. 10B) Summary layout of aggregated connection weights between modules M1-M4. Arrows show directionality of connections and their thickness is proportional to the average connection weights of between-module connections. For abbreviations see description of FIG. 6.

FIGS. 11A-11H. Topographic distribution of modules and rich club. Regions, modules, and rich club are plotted on a standard atlas of the rat brain (Swanson, L. W. (2004) Brain Maps: Structure of the Rat Brain. A Laboratory Guide with Printed and Electronic Templates for Data, Models and Schematics (Elsevier, Amsterdam), 3rd Ed.), with atlas levels (AL in FIGS. 11A-11G) arranged from rostral to caudal, as indicated in the dorsal view of the rat brain (FIG. 1111). FIG. 11A shows AL12. FIG. 11B shows AL15. FIG. 11C shows AL18. FIG. 11D shows AL20. FIG. 11E shows AL22. FIG. 11F shows AL26. FIG. 11G shows AL29. FIG. 11H shows the dorsal view of the rat brain. For clarity, the rich club is shown only on the left side of the brain; for abbreviations see the description of FIG. 6.

FIGS. 12A-12C. FIG. 12A shows module stability for connections between the cerebral nuclei in each hemisphere, displayed here for the bihemispheric 90-region network. FIGS. 12B and 12C show stable module partitions encountered in the range γ=[0.5 1.5], with regions within modules arranged by their total node strength. FIG. 12B shows a stable module partition. FIG. 12C shows a stable module partition. One partition into 4+4 modules (identical to that in FIG. 8A) remains stable across most of the parameter range. Regions in FIG. 12C are arranged in the same order as for FIG. 8A, in both hemispheres. For abbreviations see description of FIG. 6.

FIG. 13 shows layout for the 90-region network between the cerebral nuclei regions on both sides of the brain. Methodology and all conventions are as in FIG. 10A. Region (node) labels are shown only for side 1 (Left), and region positions are symmetric across the midline, with homotopic connections extending horizontally as they connect symmetrically arranged node pairs on the two sides. For abbreviations see description of FIG. 6.

FIG. 14 shows layout for association (Left) and commissural (Right) connections originating from the 45 cerebral nuclei regions in one hemisphere. Association and commissural connections originating from the cerebral nuclei regions in the other hemisphere are assumed to be symmetric in rat (FIG. 13) based on current data. All conventions are as in FIGS. 10A and 13. For abbreviations see description of FIG. 6.

FIG. 15 shows scatter plot of each region's intrahemisphere degree (the number of its distinct input plus output connections, absent any commissural connections) vs. the region's interhemisphere degree (the number of its distinct commissural connections, both homo- and heterotopic inputs plus outputs). The two measures are significantly correlated (Pearson correlation, R=0.619, P=5.84×10⁻⁶; Spearman correlation, R=0.641, P=2.14×10⁻⁶). For abbreviations see description of FIG. 6.

FIGS. 16A and 16B show distribution of weight categories of ipsilateral macroconnections (FIG. 16A) and weight scale used for weighted network analysis (FIG. 16B).

FIG. 17 shows rankings of regions according to four centrality measures: degree, strength, betweenness, and closeness. Regions are ranked by degree across all four plots, with the top 20th percentile for each measure colored in grey.

FIGS. 18A and 18B show rich club organization. (FIG. 18A) Plots of the weighted rich club coefficient of the empirical network (dark grey curve) and the mean (light grey curve) and mean±SD (dashed light grey curves) for a population of randomized networks. (FIG. 18B) Normalized rich club coefficient across node degree, with statistically significant (after false discovery rate correction) data points shown in black.

FIG. 19 shows rat intracerebral nuclei connectome. Directed synaptic macroconnectome matrix with gray matter region sequence (Left, Top to Bottom, list of macroconnection origins/from; Top, Left to Right, same list of macroconnection terminations/to) in the Brain Maps 4 (Table 4) topographic nomenclature hierarchy. Ipsilateral connections are shown in Upper Left and Lower Right, whereas contralateral connections are shown in Upper Right and Lower Left. The grey diagonal lines show homotopic commissural connections, that is, a connection arising from a region of interest on one side and terminating in the same region of interest on the other side. Use of these values for network analysis is described in the Materials and Methods section below.

### DETAILED DESCRIPTION

It is to be understood that this invention is not limited to particular embodiments described, as such may of course vary. It is also to be understood that the terminology used herein is for the purpose of describing particular embodiments only, and is not intended to be limiting, since the scope of the present invention will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

### Definitions

Unless defined otherwise, all technical and scientific terms used herein have the same meaning as commonly understood by one of ordinary skill in the art to which this invention belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The use of the term “or” in the claims is used to mean “and/or” unless explicitly indicated to refer to alternatives only or the alternatives are mutually exclusive, although the disclosure supports a definition that refers to only alternatives and “and/or.”

The term “about” when used before a numerical designation, e.g., temperature, time, amount, and concentration, including range, indicates approximations which may vary by (+) or (−) 10%, 5%, or 1%. “About” may indicate that a value includes the standard deviation of error for the device or method being employed to determine the value.

As used herein, the term “nervous system” refers to the central nervous system, the peripheral nervous system, or both the central and peripheral nervous systems. The nervous system coordinates an organism's actions by transmitting signals to and from different parts of its body. The functions of the nervous system include but are not limited to sensory division, motor division, sympathetic division, impulse control via the autonomic nervous system, parasympathetic division, and somatic motor control. The central nervous system is comprised of the brain and spinal cord and it houses the integrative and control centers. The peripheral nervous system is comprised of cranial nerves and spinal nerves and functions to communicate between the CNS and the rest of the body.

As used herein, the term “nervous system data” refers to data produced by or collected from the nervous system or a part of the nervous system. Such data includes but is not limited to cerebral nuclei data, gene expression data, protein expression data, proteomic data, genetic data, expression of neural cell biomarkers, expression or detection of neurotransmitters, neurotransmitter receptors, and cellular markers, brain imaging data including magnetic resonance imaging and computed tomography data, and neural circuit data. In some embodiments, the data is produced by or collected from the CNS. In other embodiments, the data is produced by or collected from the PNS. In further embodiments, the data is produced by or collected from the CNS or PNS. In some embodiments, nervous system data is mined from a search of the relevant literature.

As used herein, “gene expression” refers to the process by which polynucleotides are transcribed into RNA such as mRNA and/or the process by which a transcribed mRNA is subsequently translated into peptides, polypeptides, or proteins. If the polynucleotide is derived from genomic DNA, expression may include splicing of the mRNA in an eukaryotic cell. Gene expression data, including detection of gene expression products, may be measured by any method known in the art including but not limited to PCR, quantitative PCR, gene expression array (e.g. gene expression chip or microarray), sequencing including high throughput sequencing, immunohistochemistry, immunofluorescence, immunoprecipitation, Western blot, Northern blot, chromatin immunoprecipitation, mass spectrometry, flow cytometry, ELISA, and high performance liquid chromatography.

Classes of cells of the central nervous system include but are not limited to neurons, excitable cells which process information, and glia, which provide the neurons with mechanical and metabolic support. Three general categories of neurons are commonly recognized including receptors, which are highly specialized neurons that act to encodesensory information, interneurons, which send and receive signals to/from other nerve cells, and effectors or motor neurons which send signals to the muscles and glands of the body, effecting behavior. Principal neurons are long-axoned cells that transmit information over long distances and provide pathways of communication within the nervous system. Local circuit neurons lack long axons and perform integrative and modulating functions in local brain regions.

Neurons are found in the brain, the vertebrate spinal cord, the invertebrate ventral nerve cord and the peripheral nerves. Neurons can be identified by expression of a number of markers that are listed herein. Antibodies and other detection reagents to identify expression of these markers are available through, for example, EMD Millipore (Massachusetts, USA) and Abcam (Cambridge, United Kingdom). For example, neurons may be identified by expression of neuronal markers B-tubulin III (neuron marker, Millipore), Tuj1 (beta-III-tubulin); MAP-2 (microtubule associated protein 2, other MAP genes such as MAP-1 or -5 may also be used); anti-axonal growth clones; ChAT (choline acetyltransferase (motoneuron marker, Millipore); Olig2 (motorneuron marker, Millipore, Chemicon), Olig2 (Millipore), CgA (anti-chromagranin A); DARRP (dopamine and cAMP-regulated phosphoprotein); DAT (dopamine transporter); GAD (glutamic acid decarboxylase); GAP (growth associated protein); anti-HuC protein; anti-HuD protein; alpha-internexin; NeuN (neuron-specific nuclear protein); NF (neurofilament); NGF (nerve growth factor); gamma-NSE (neuron specific enolase); peripherin; PH8; PGP (protein gene product); SERT (serotonin transporter); synapsin; Tau (neurofibrillary tangle protein); anti-Thy-1; TRK (tyrosine kinase receptor); TRH (tryptophan hydroxylase); anti-TUC protein; TH (tyrosine hydroxylase); VRL (vanilloid receptor like protein); VGAT (vesicular GABA transporter), VGLUT (vesicular glutamate transporter). Cellular markers specific for intermediate progenitor cells include TBR2 and MASH1/Ascl1. Cellular markers specific for immature neurons include Doublecortin, beta III tubulin, NeuroD1, TBR1, and stathmin 1. Cellular markers specific for mature neurons include NeuN, MAP2, 160 kDa neurofilament medium, 200 kDa neurofilament heavy, synaptophysin, and PSD95. Cellular markers specific for glutamatergic neurons include vGluT1, vG1, uT2, NMDAR1, NMDAR2B, glutaminase, and glutamine synthetase. Cellular markers specific for GABAergic neurons include but are not limited to GABA transporter 1, GABAB receptor 1, GABAB receptor 2, GAD65, and GAD67. Cellular markers specific for dopaminergic neurons include but are not limited to Tyrosine hydroxylase, dopamine transporter, FOXA2, GIRK2, Nurr1, LMX1B. Cellular markers specific for serotonergic neurons include but are not limited to Tryptophan hydroxylase, serotonin transporter, and Pet1. Cellular markers specific for cholinergic neurons include but are not limited to Choline acetyltransferase, vesicular acetylcholine transporter, and acetylcholinesterase.

Glial cells are non-neuronal cells that function to surround neurons and provide positional support, supply neurons with nutrients and oxygen, insulate neurons from other cells, and remove pathogens and damaged neurons. In the CNS, glial cells include oligodendrocytes, astrocytes, ependymal cells and microglia, and in the PNS, glial cells include Schwann cells and satellite cells. Cellular markers specific for radial glia include vimentin, PAX6, HES1, HES5, GFAP, EAAT1/GLAST, BLBP, TN-C, N-cadherein, Nestin, and SOX2. Cellular markers specific for oligodendrocyte precursor cells include PDGFRA and NG2. Cellular markers specific for oligodendrocytes include Olig1, Olig2, Olig3, OSP, MBP, MOG, and SOX10. Cellcular markers specific for astrocytes include but are not limited to GFAP, EAAT1/GLAST, EAAT2/GLT-1, Glutamine synthetase, S100 beta, ALDH1L1. Cellular markers specific for Microglia include CD11b, CD45, Iba1, F4/80, CD68, and CD40. Cellular markers specific for non myelinating schwann cells include SOX10, S100, GAP43, NCAM, and P75NTR. Cellular markers for myelinating schwann cells include SOX10, S100, EGR2, MBP, and MPZ. Cellular markers specific for Schwann cell precursors include SOX10, GAP43, BLBP, MPZ, Dhh, P75NTR. Cellular markers specific for Neuroepithelial cells include Nestin, SOX2, Notch1, HES1, HES3, Occludin, E-cadherin, and Sox10.

A neurotransmitter is a chemical messenger that enables neurotransmission across a chemical synapse. Examples of neurotransmitters include but are not limited to adrenaline, noradrenaline, dopamine, serotonin, GABA, acetylcholine, glutamate, aspartate, opioids, and endorphins. A neurotransmitter receptor is a membrane receptor that recognizes and is activated by a neurotransmitter. The two major types of neurotransmitter receptors include ligand-gated receptors and G protein-coupled receptors. Examples of neurotransmitter receptors include but are not limited to: α1A, α1b, α1c, α1d, α2a, α2b, α2c, α2d, β1, β2, and β3 adrenergic receptors; D1, D2, D3, D4, and D5 dopaminergic receptors; GABA_{A}, GABA_{B1a}, GABA_{B1δ}, GABA_{B2}, and GABA_{C} GABAergic receptors; NMDA, AMPA, kainate, mGluR1, mGluR2, mGluR3, mGluR4, mGluR5, mGluR6, and mGluR7 glutaminergic receptors; H1, H2, and H3 histaminergic receptors; Muscarinic: M1, M2, M3, M4, M5: Nicotinic: muscle, neuronal (α-bungarotoxin-insensitive), and neuronal (α-bungarotoxin-sensitive) cholinergic receptors; μ, δ1, δ2, and κ opioid receptors; 5-HT1A, 5-HT1B, 5-HT1D, 5-HT1E, 5-HT1F, 5-HT2A, 5-HT2B, 5-HT2C, 5-HT3, 5-HT4, 5-HT5, 5-HT6, and 5-HT7 serotonergic receptors; and glycinergic receptors.

As used herein, a “processor” may comprise any suitable device that provides processing, storage, and input/output devices executing application programs and the like. Exemplary processors may be implemented in integrated circuits, field-programmable gate arrays, and/or any other suitable architecture. In some embodiments, processors are linked through communications networks to other computing devices, including other processors and/or server computer(s). In some embodiments, the communications network are part of a remote access network, a global network (e.g., the Internet), a worldwide collection of computers, Local area or Wide area networks, and gateways that currently use respective protocols (TCP/IP, Bluetooth, etc.) to communicate with one another. Other electronic device/computer network architectures are also suitable.

As used herein, “memory” refers to a non-transitory computer-readable storage medium having computer code thereon for performing various computer-implemented operations. Such non-transitory computer-readable storage medium is any medium that is capable of storing or encoding a sequence of instructions or computer codes for performing the operations, methodologies, and/or techniques described herein. Examples of memory include, but are not limited to discs such as internal hard drives, removable hard drives, magneto-optical, CD, DVD, and Blu-ray discs, memory sticks, and other hardware devices that are specially configured to store and execute program code, such as ASICs, FPGAs or programmable logic devices (PLDs); semiconductor devices such as RAM, ROM, EPROM, EEPROM, and flash memory devices; and cloud-based storage wherein the memory is stored in logical pools accessible through a co-located cloud computer service, a web service application programming interface or by applications that utilize the application programming interface (e.g. cloud storage gateway, cloud desktop storage, or Web-based content management systems). The physical storage for cloud data can be located across multiple servers.

### Descriptive Embodiments

This disclosure provides a system and method for modeling connections, structure, and/or function of biological systems such as the the nervous system including the CNS and/or PNS. In one aspect, the method comprises generating, with a processor, a plurality of connections corresponding to CNS and/or PNS data, wherein each of the plurality of connections comprises an origin, a termination, and a degree of connection. The method also includes storing the plurality of connections in a first memory location, receiving a nervous system atlas, and storing the nervous system atlas in a second memory location. The method also includes matching, with a processor on a connection-by-connection basis, each origin and each termination of the plurality of connections to a corresponding position of the nervous system atlas to produce an annotated connection matrix. The method further includes converting the annotated connection matrix to one or more modules, wherein the one or modules comprise an aggregated ranking of connections exceeding a threshold.

Axiome, a set of exemplary information science (informatics) tools, can be implemented in various forms such as supported by an in-development guide and supporting website, and involving highly structured, systematized, and interactive spreadsheet templates for use with Microsoft Excel, and designed primarily for data entry, collation, and comparison of neuroscience information data (neuroinformatics). In some embodiments, Axiome may be used on a different spreadsheet platform such as Google Sheets (part of the Google Drive suite). In some aspects, Axiome is a platform or software tool. Axiome M, Axiome C, and four supporting/accessory tools are described herein.

More specifically, the tools are designed to mesh with mapping of neuroscience brain data obtained from research using the rat as a research animal to a specific reference brain or nervous system atlas (e.g., Brain Maps 4 beta version, by Larry W. Swanson, available Open Access under a Creative Commons Attribution-No Commercial 4.0 International License at larrywswanson.com/?page_id=901,). The nervous system atlas may be an atlas of any part or the whole of the nervous system of an organism such as the brain or a subregion of the brain. The tools include various features available within Excel or an equivalent platform, including (but not limited to) Data Validation, and Conditional Formatting rules to reduce data entry errors while providing an intuitive and user-friendly interface for the end user.

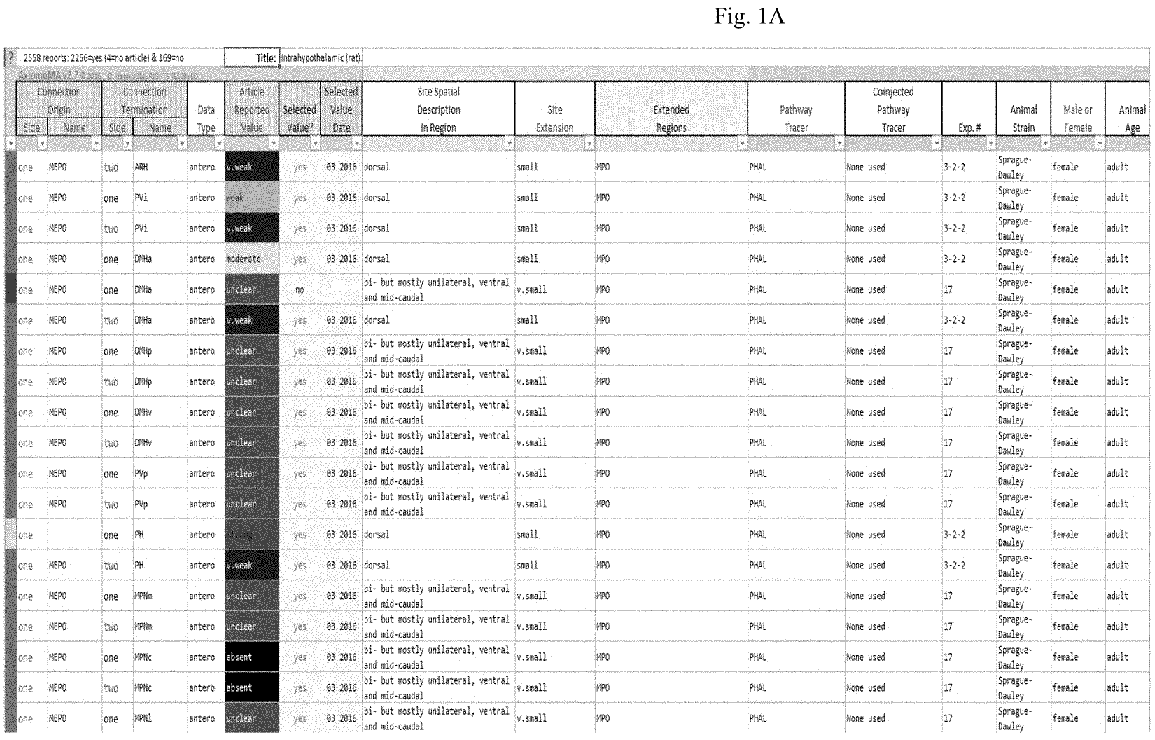

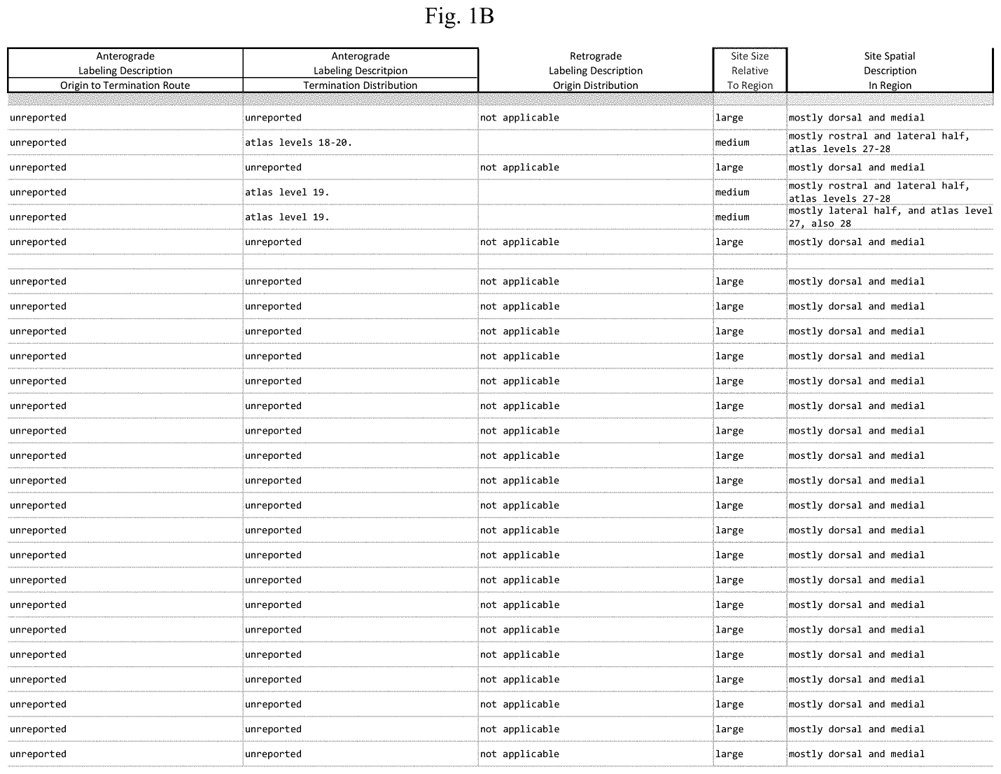

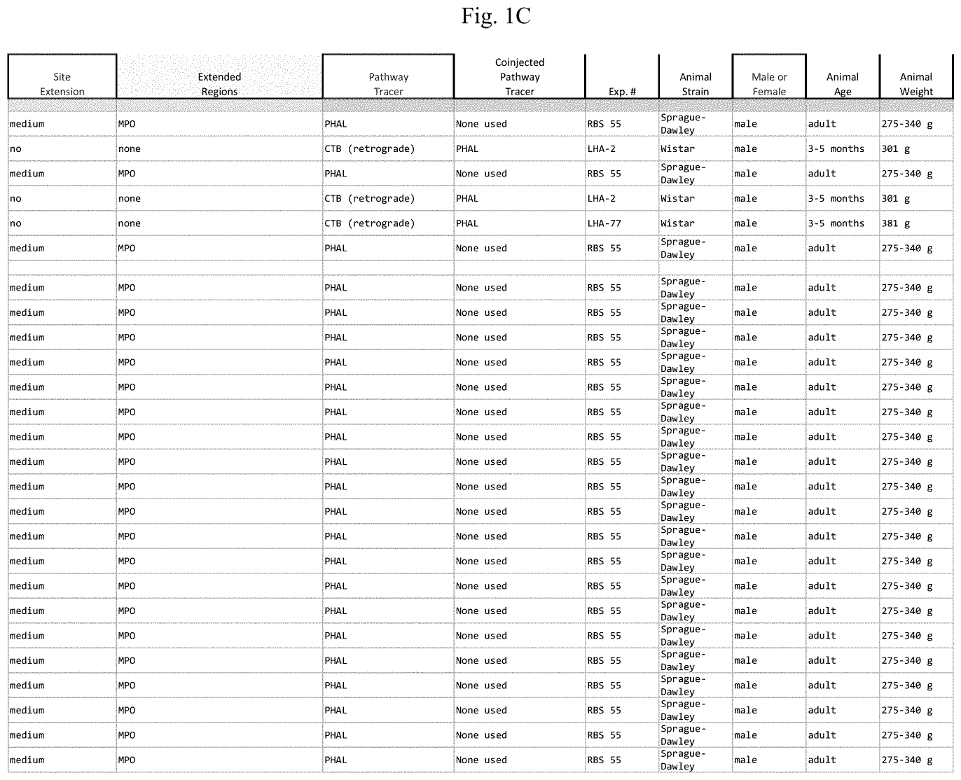

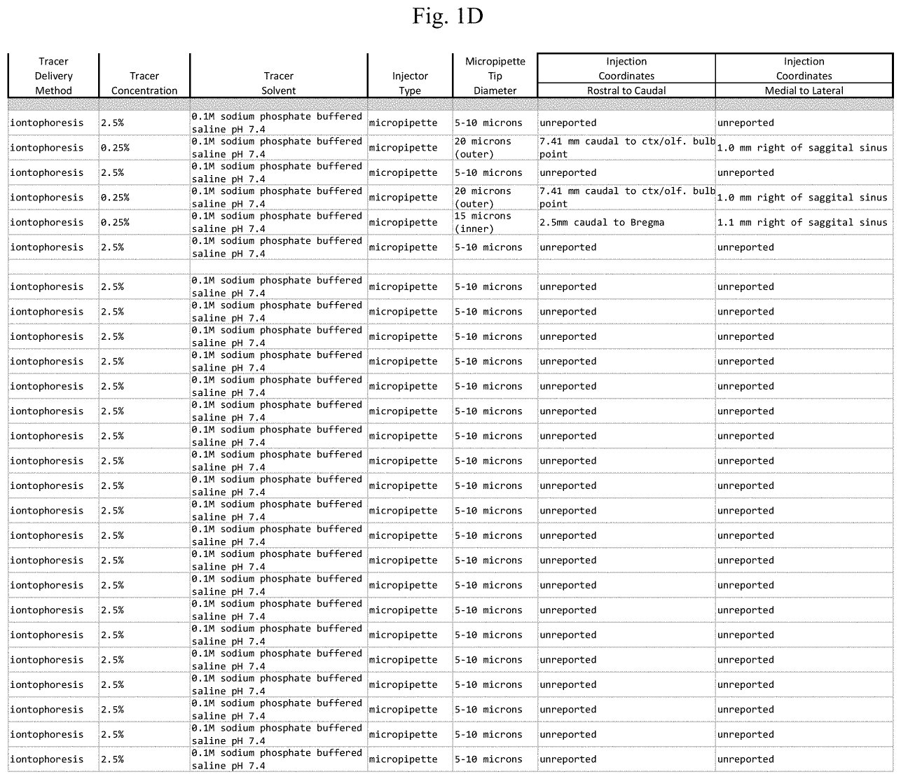

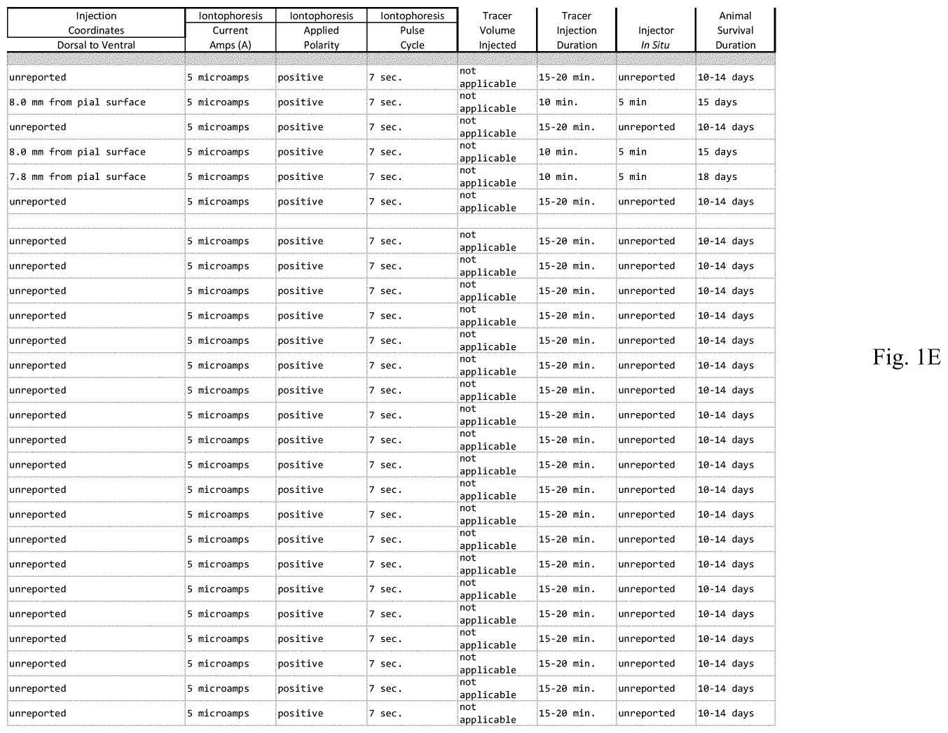

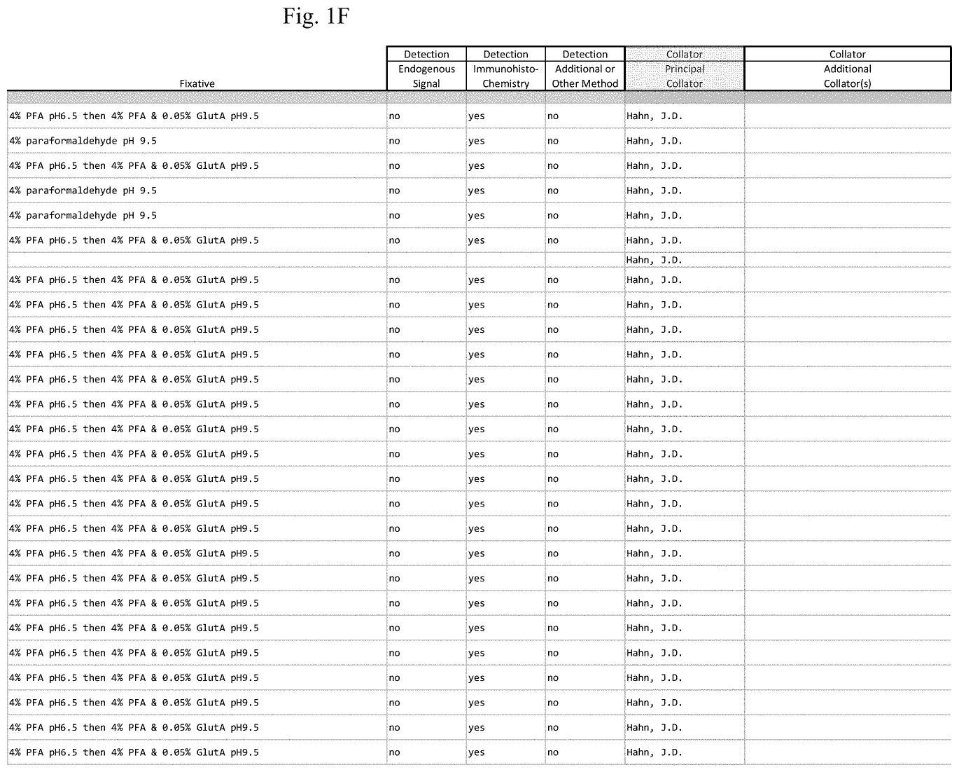

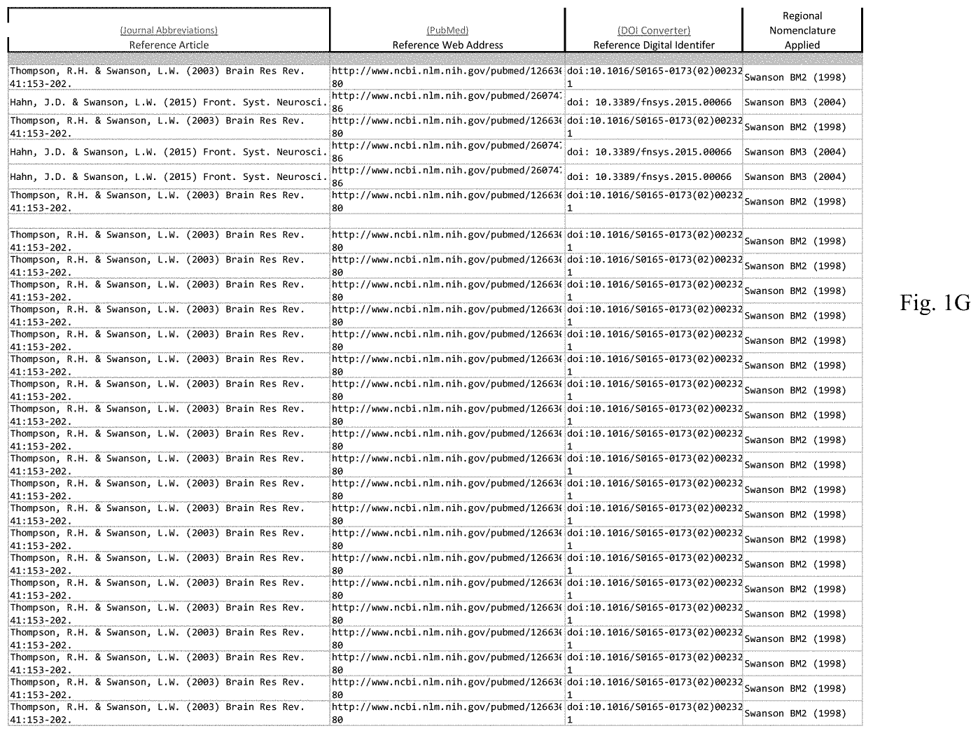

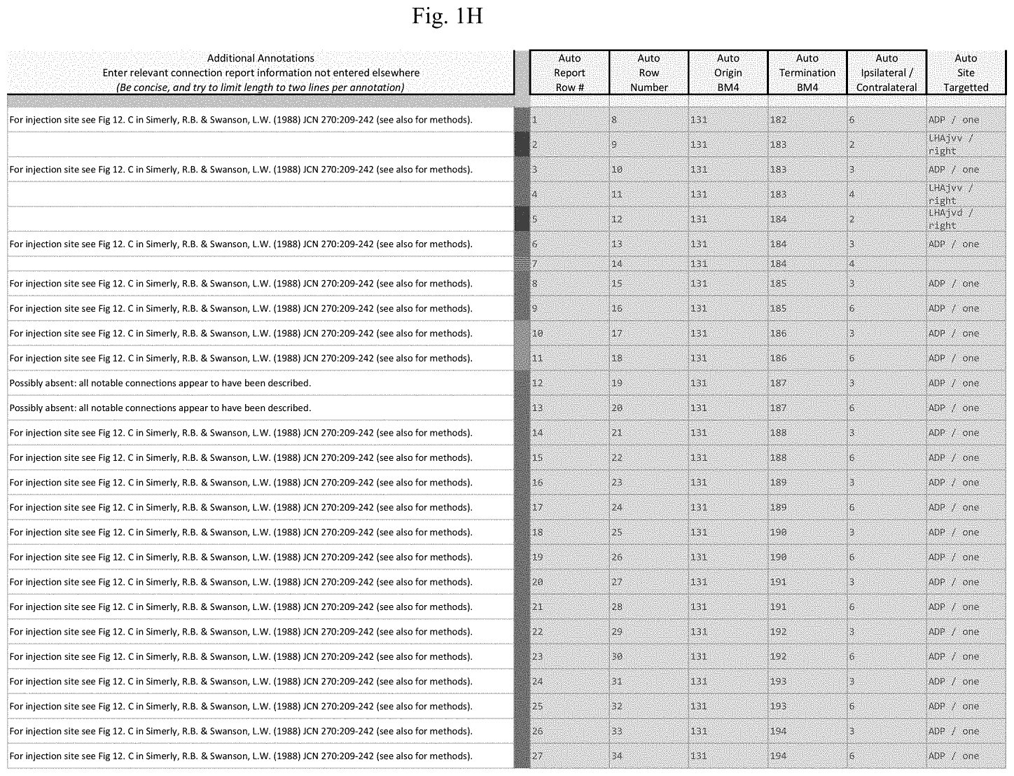

In one embodiment, as illustrated in FIGS. 1A-1I, the Axiome tool, M template (“Axiome M”) facilitates entry, collation, and comparison of brain connection data. Specifically, Macroconnection data (hence the “M” in Axiome M) that is data on connections between brain regions identified and defined by their cellular (or cyto) architecture. The Axiome M template was used to acquire the dataset that formed the basis of a recent original research on network architecture of the cerebral nuclei (basal ganglia) association and commissural connectome.

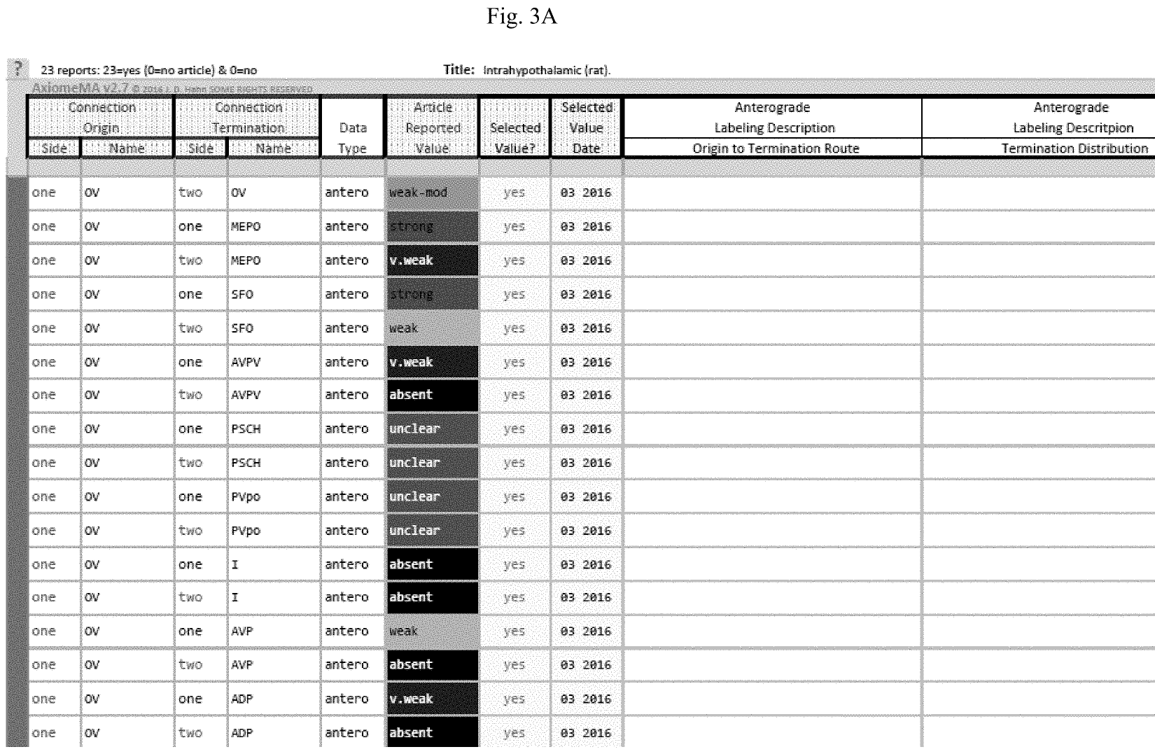

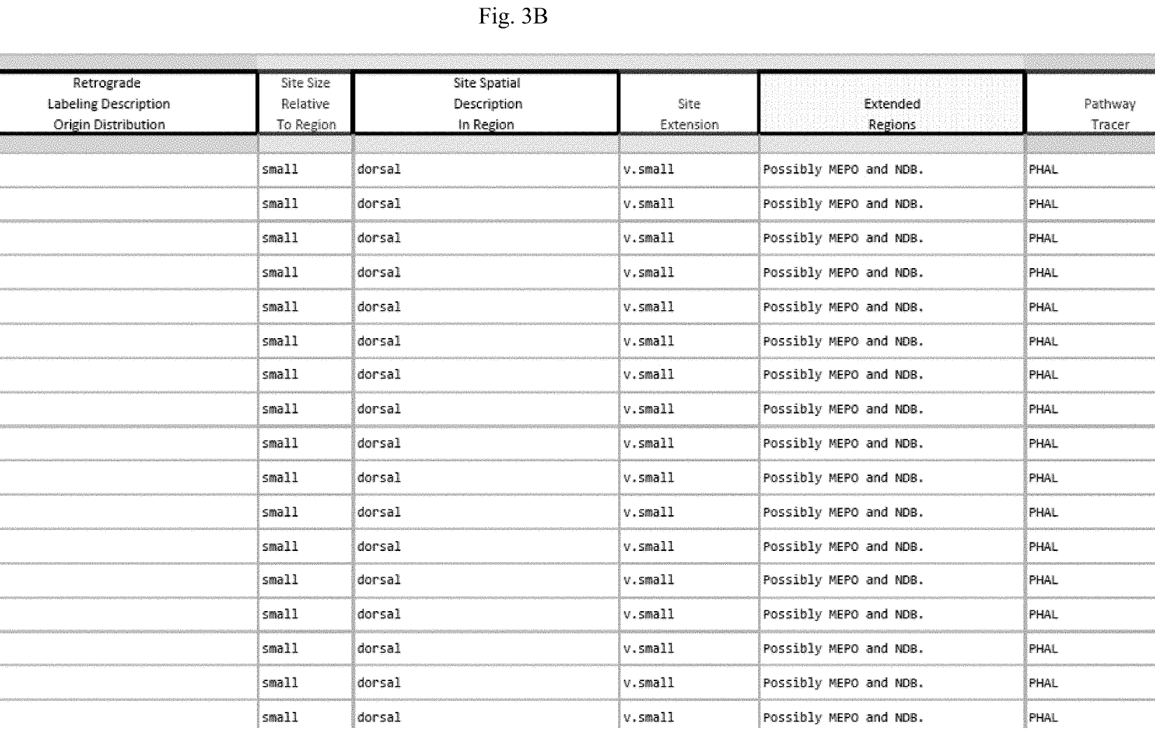

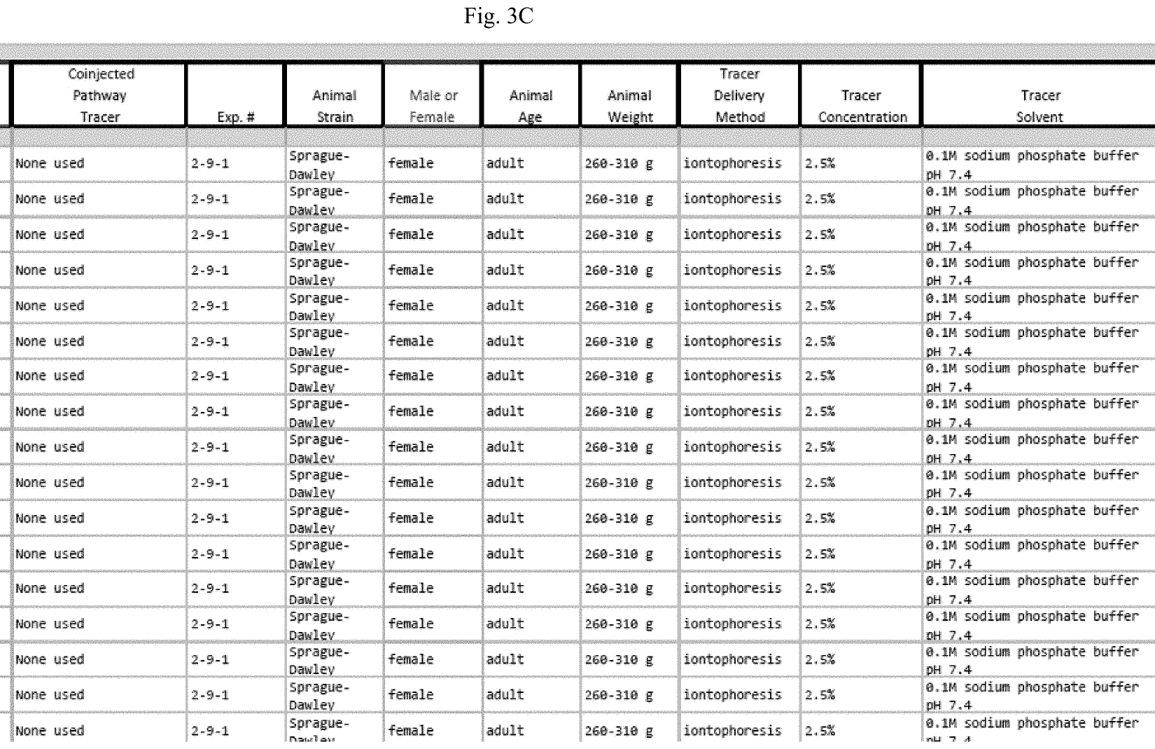

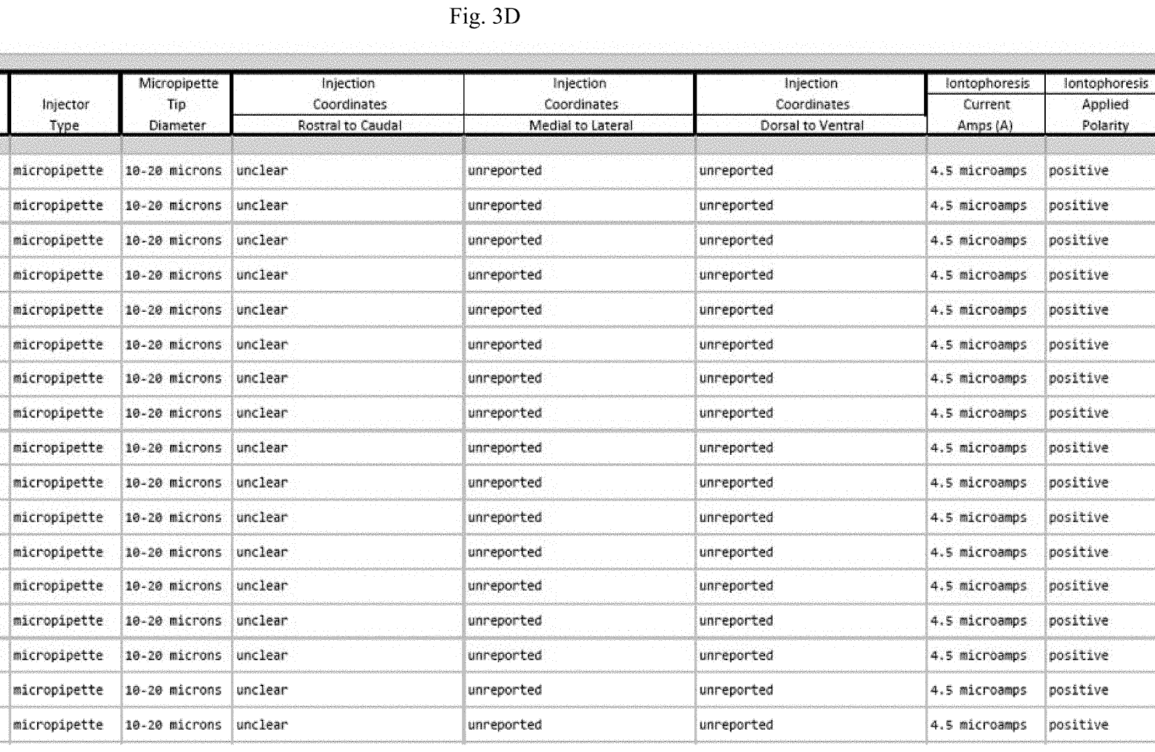

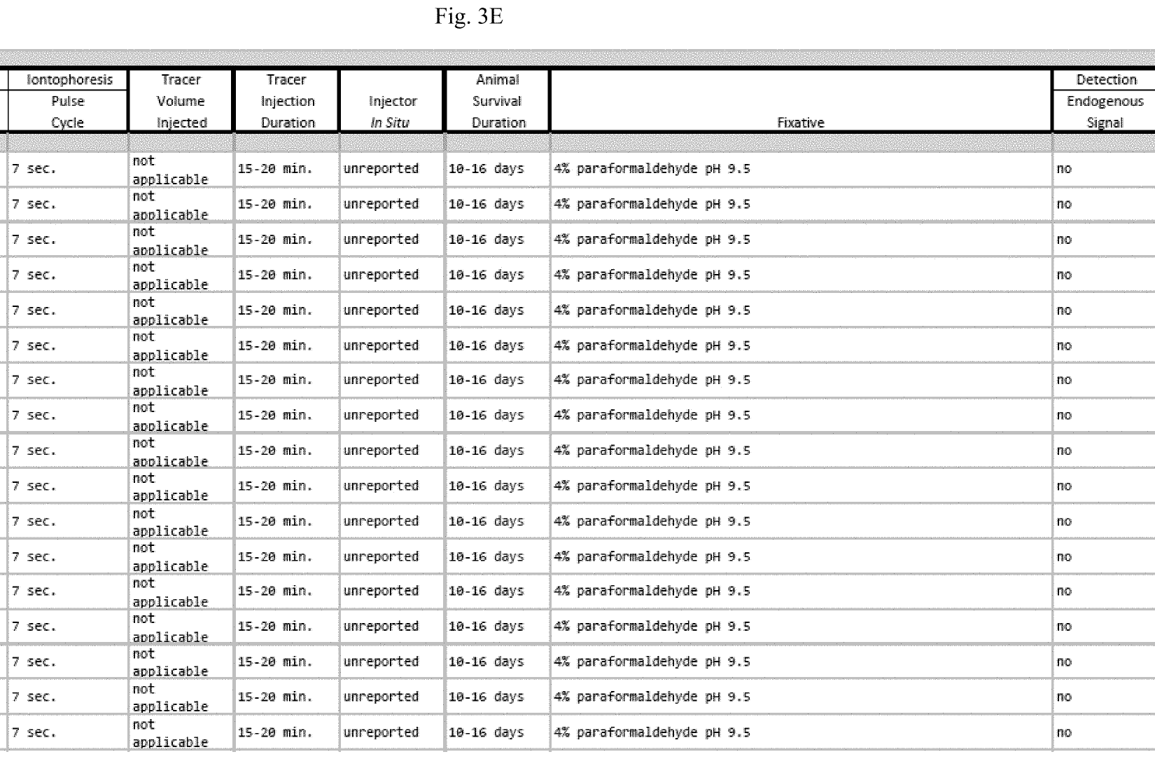

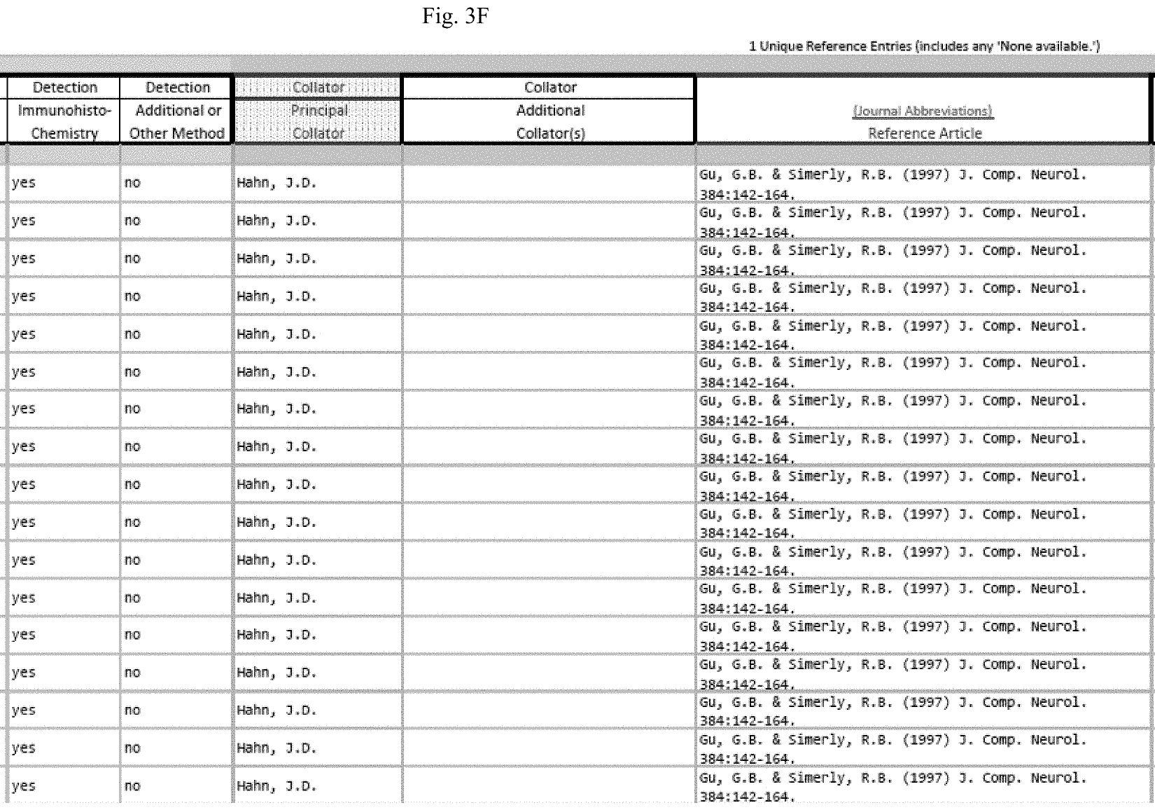

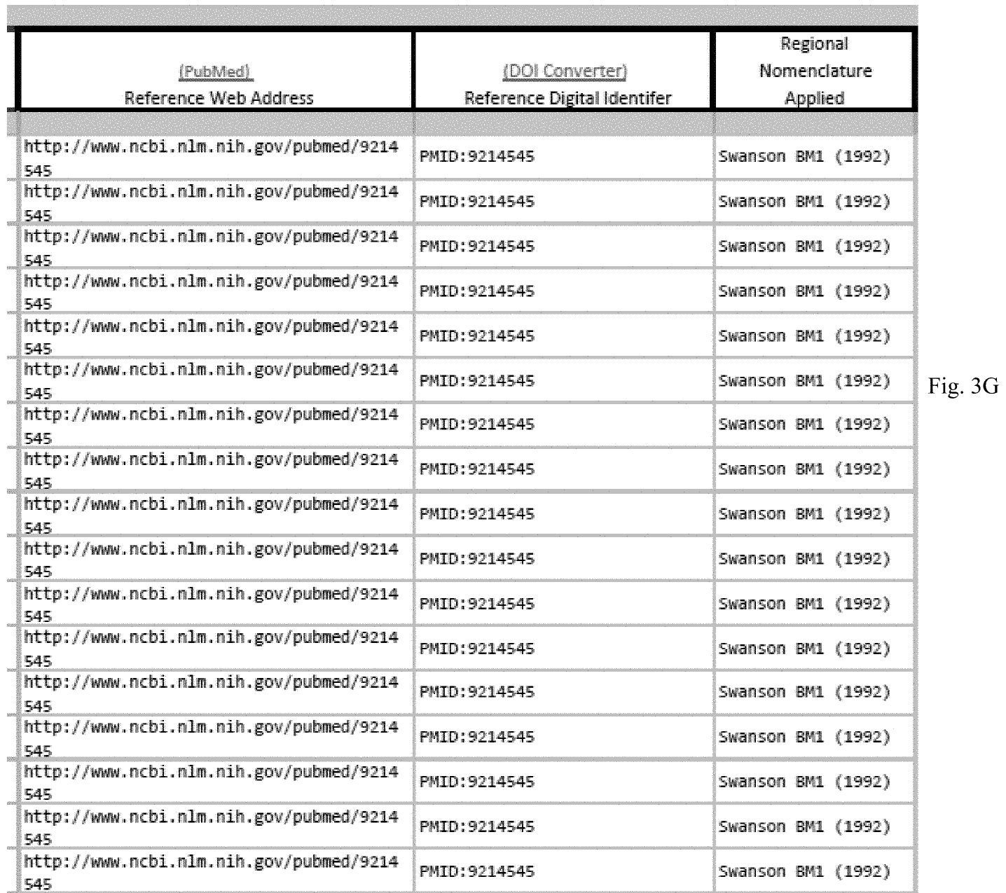

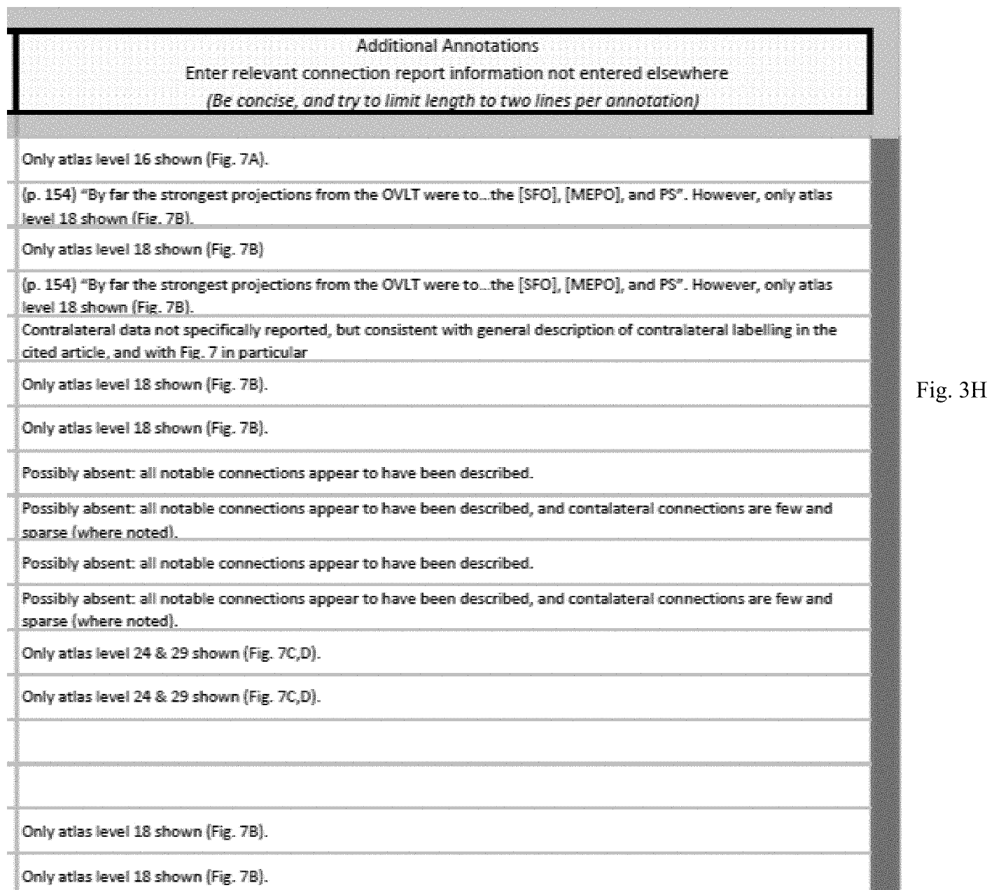

As illustrated in FIGS. 3A-3H, in addition to basic connection data (origin and termination sites), several other exemplary values may be entered in a highly structured fashion (each using a separate data column). The template incorporates several exemplary calculation features combined with a graphical user interface that provides real-time feedback, including a count of the number of connection reports and their relative value, as well as identifying different types of reports, and incomplete reports. Axiome M combines combinatorial analysis of multi-criteria data matrices generated from Axiome M datasets.

In other embodiments, the Axiome M platform can be used for mapping mesoconnections (connections between different cell types). Axiome M can also be modified for mapping brain data in multiple species and to collate data for other networks (such as social networks, transport networks, or communications networks).

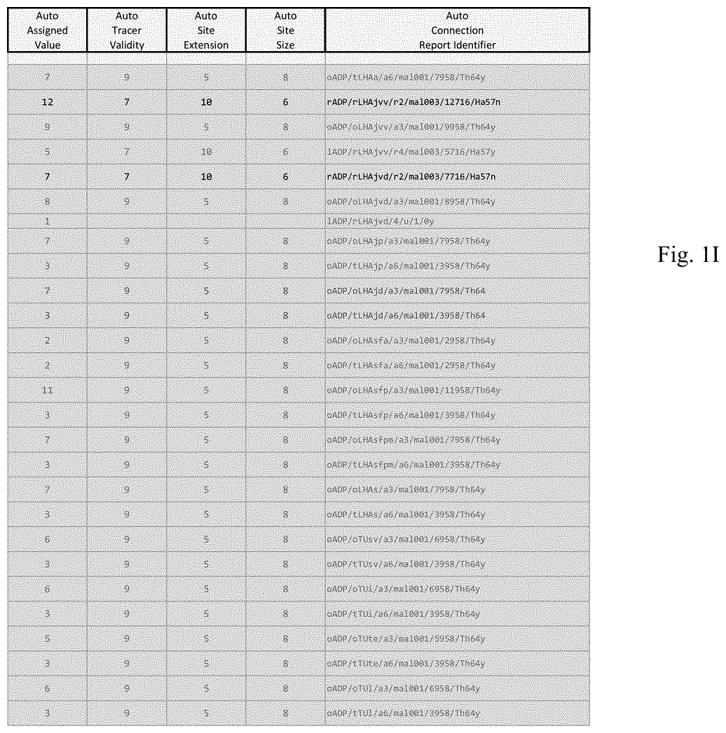

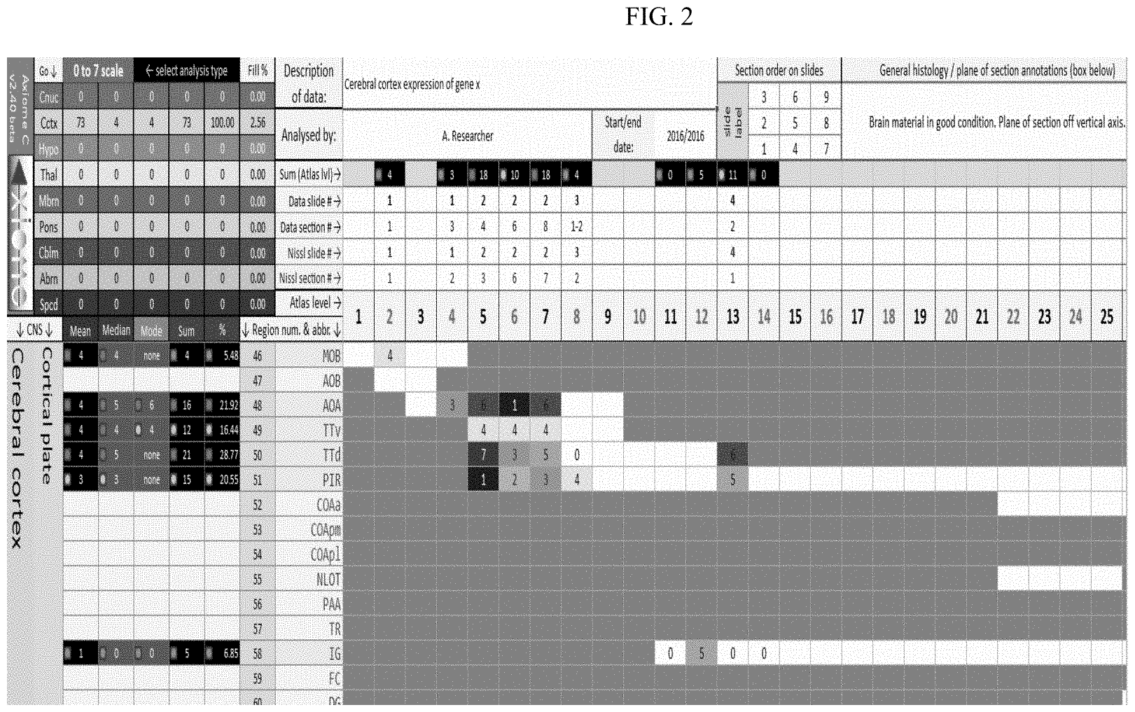

In another embodiment, as illustrated in FIG. 2, the Axiome tool, C (“Axiome C”) template facilitates entry, collation, and comparison of brain expression data. Specifically, qualitative cellular Chemoarchitecture or quantitative Count data (hence the “C” in Axiome C) that is data on (for example) the expression of a particular gene, neurotransmitter, neurotransmitter receptor, or any other cellular marker within brain regions identified and defined by their cellular (or cyto) architecture. Because of the wide diversity of data that may be entered in Axiome C, it could be used for analysis and modeling of brain data derived from a wide range of experimental models, including (but not limited to) the following 3 examples: 1) drug interactions in relation to specific diseases, in terms of how these effect changes in gene expression patterns within the brain; 2) analysis of molecular markers of neuronal cell death in studies of neurodegenerative diseases; 3) mapping of changes in the levels of biomarkers for a host of different disease states, such as diabetes, obesity, neuropsychiatric disorders.

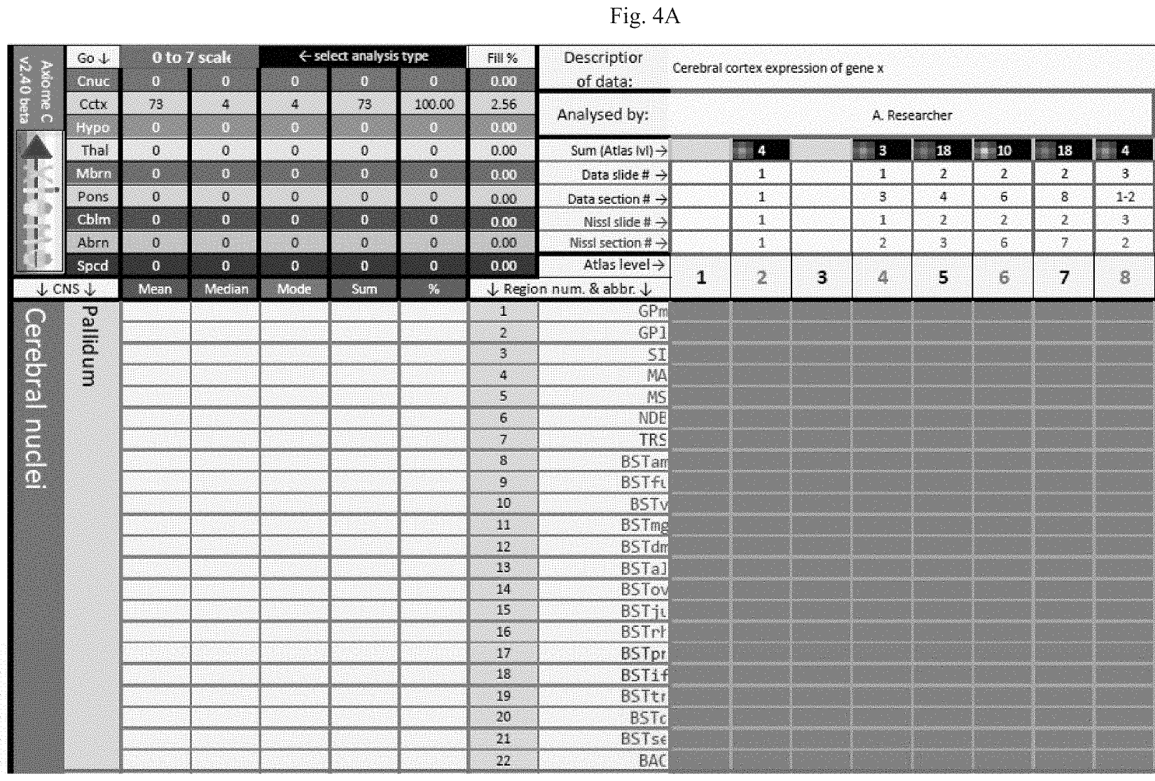

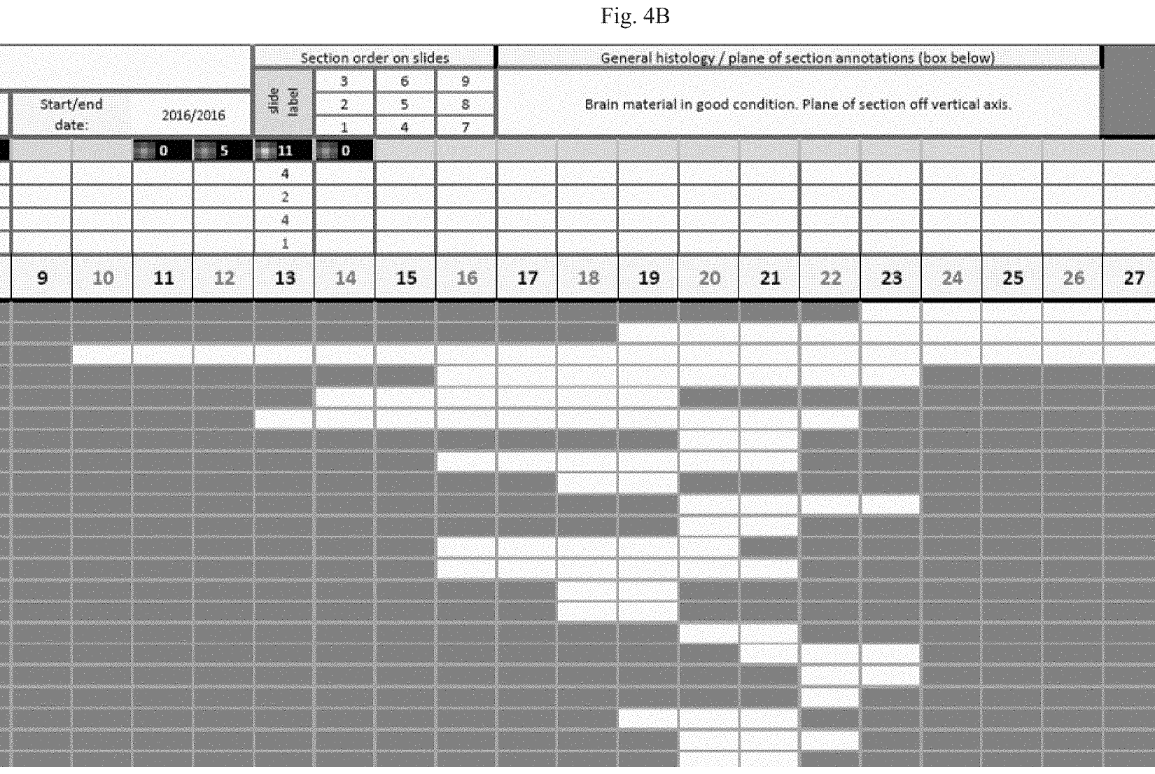

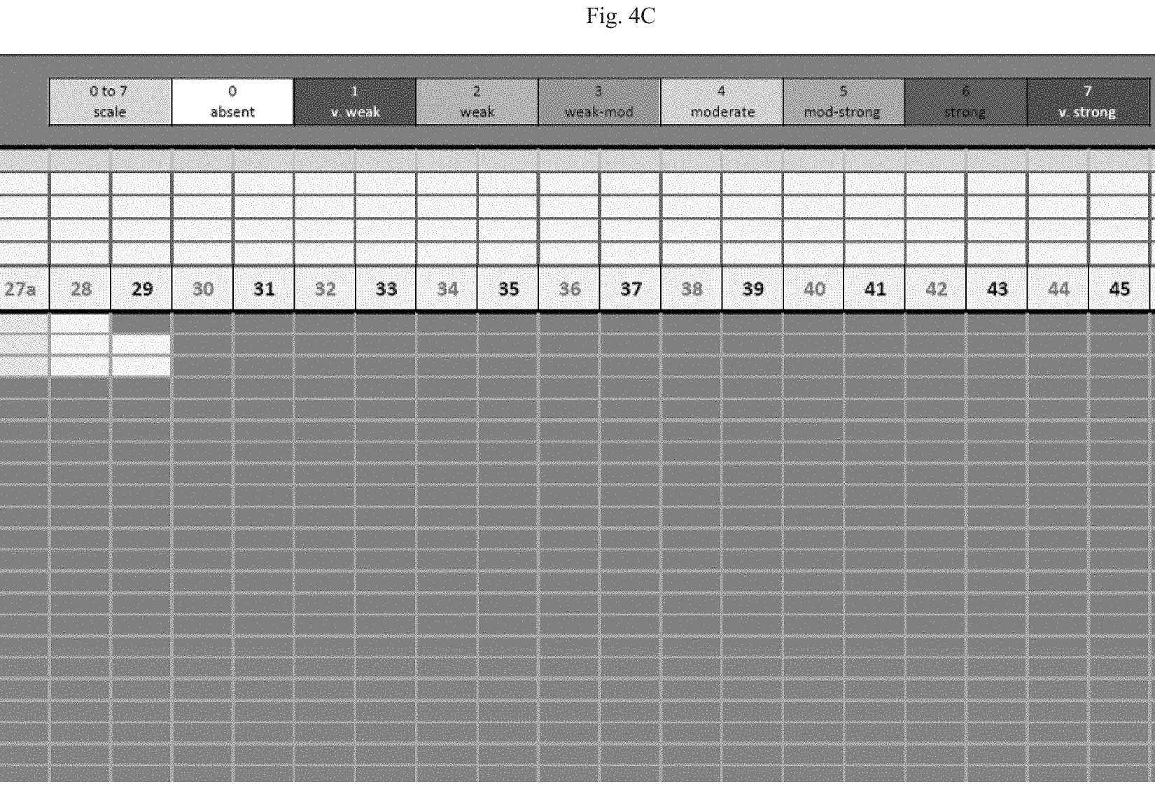

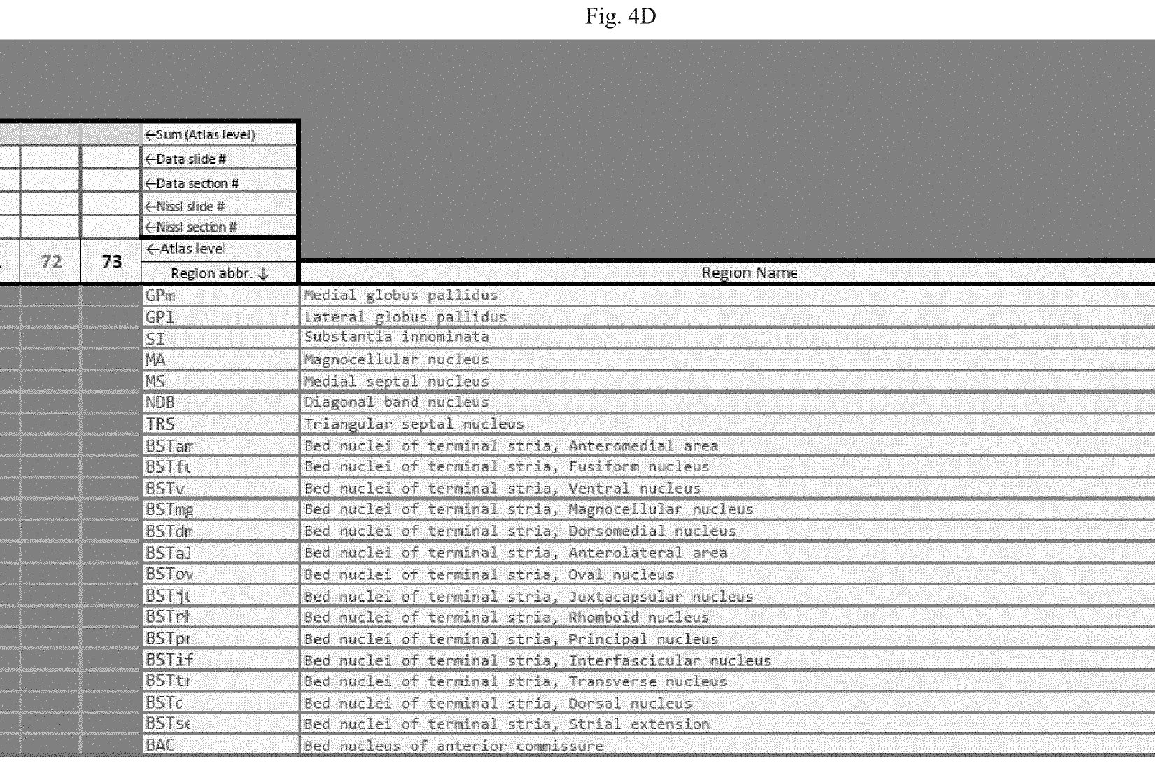

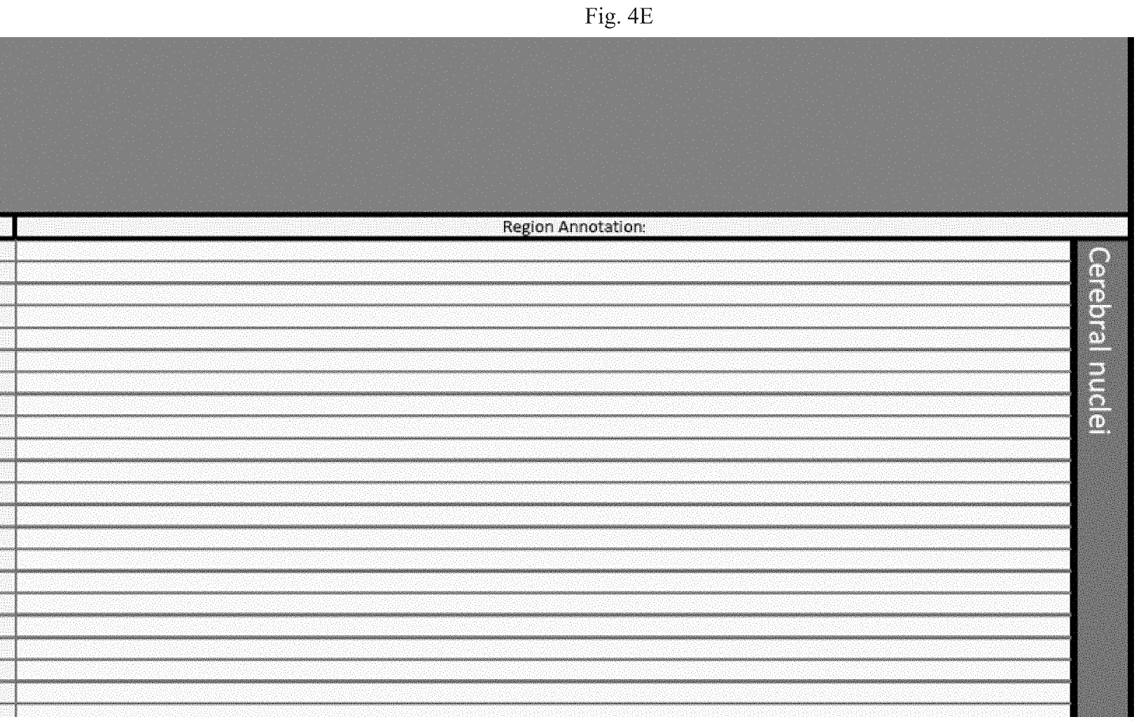

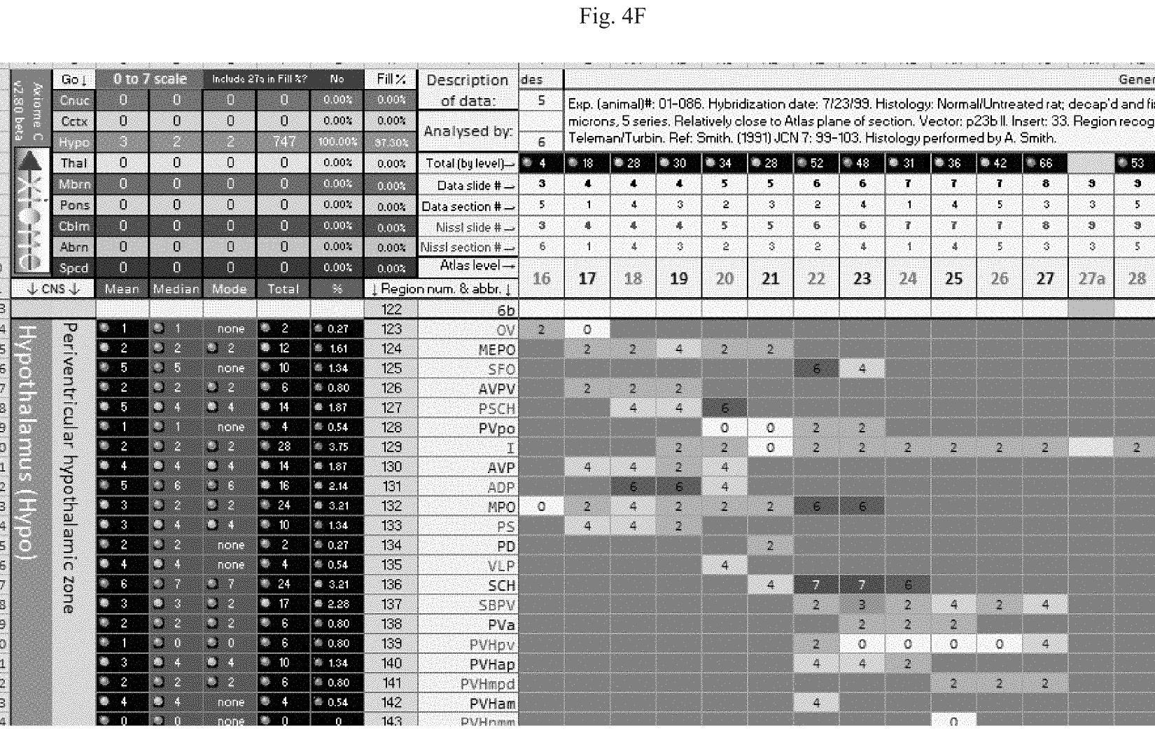

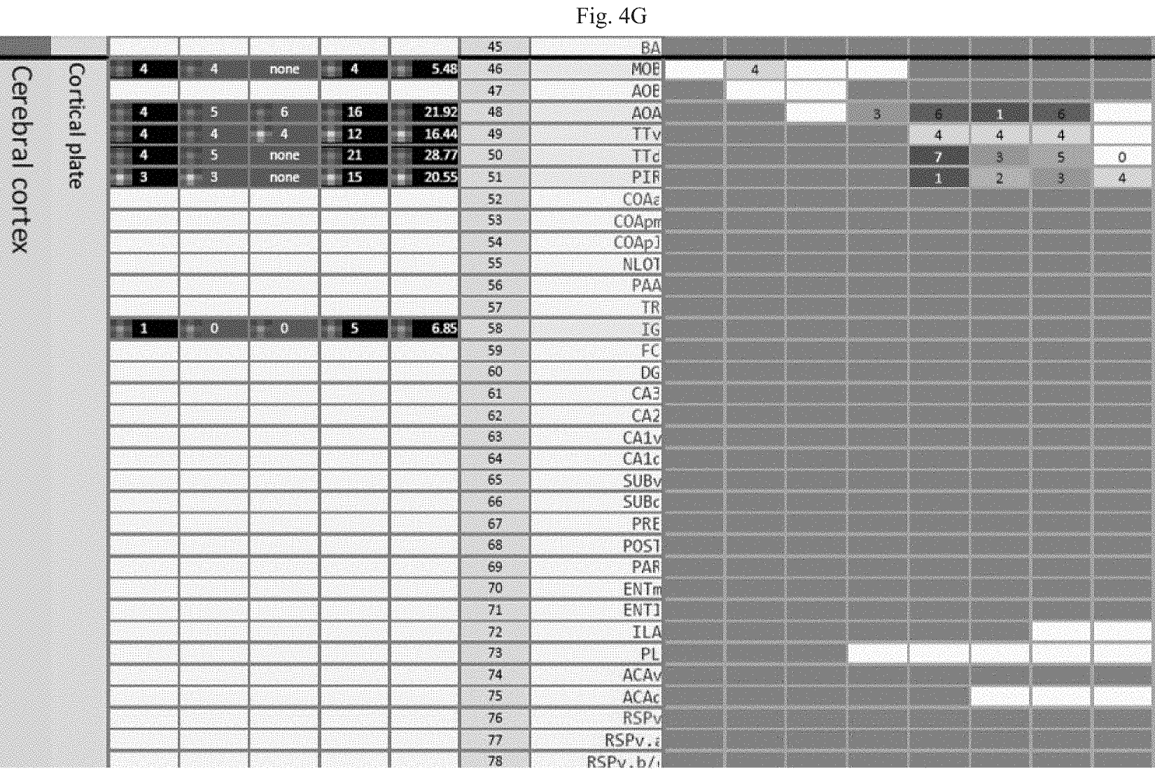

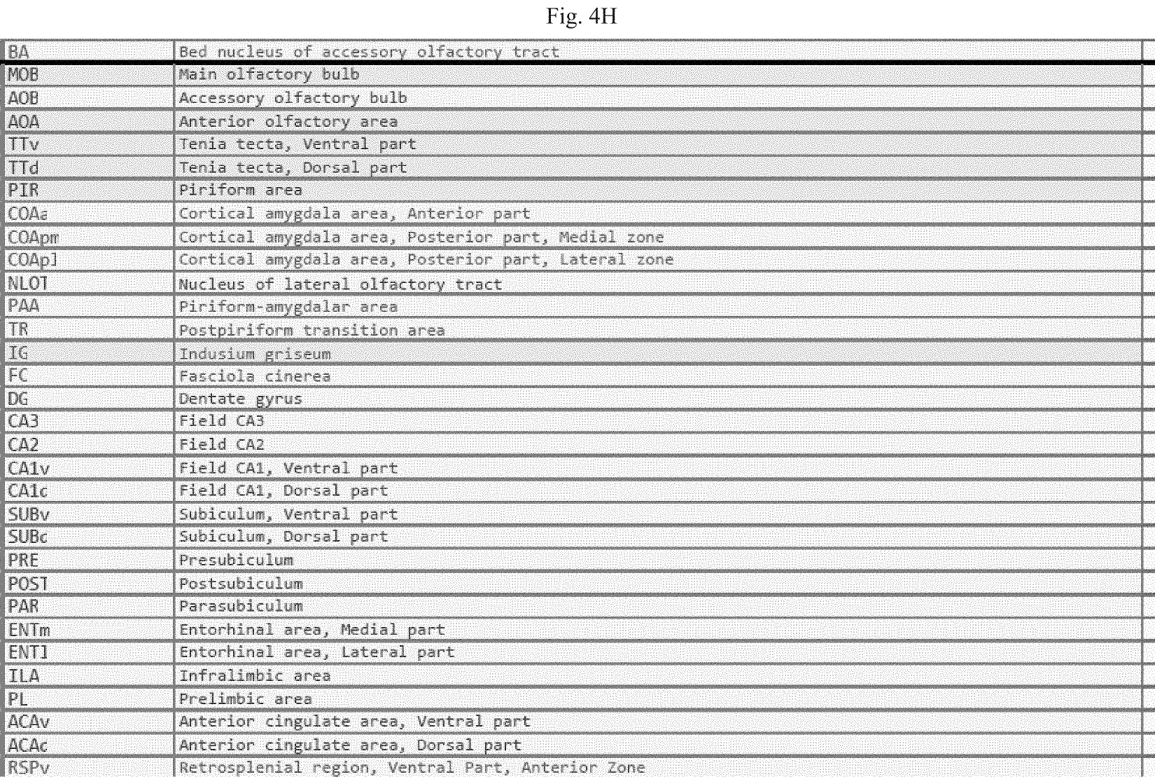

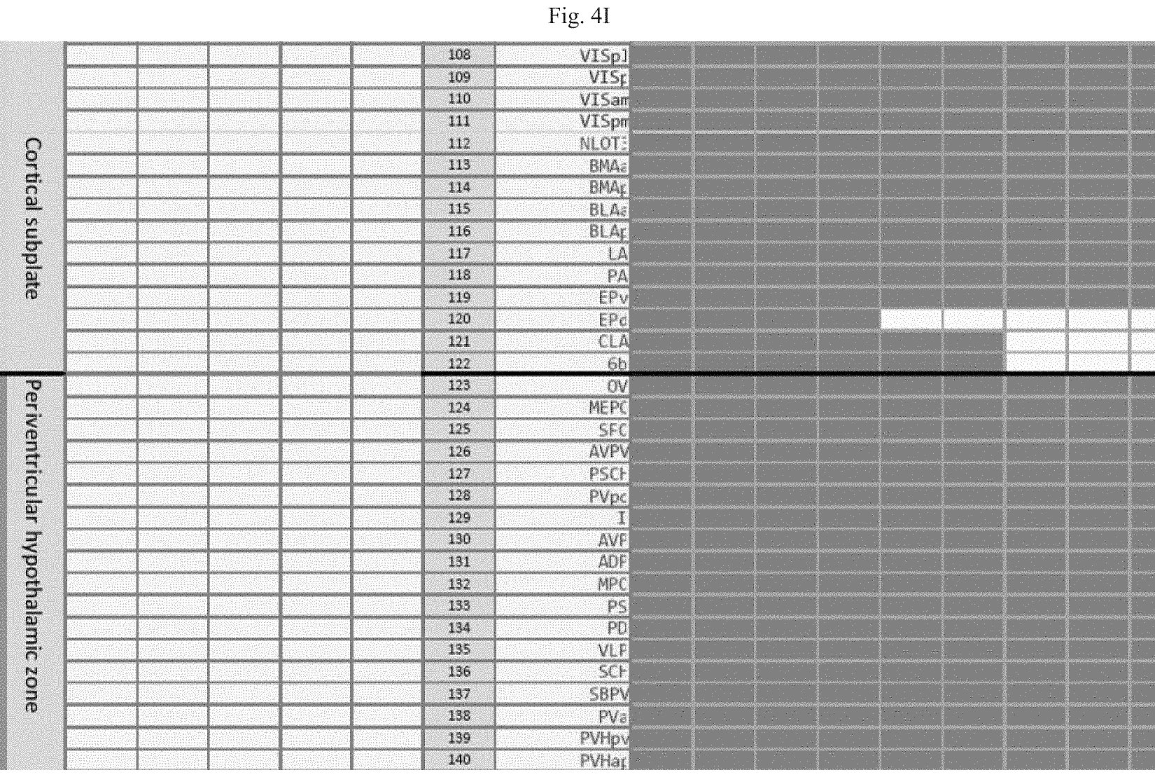

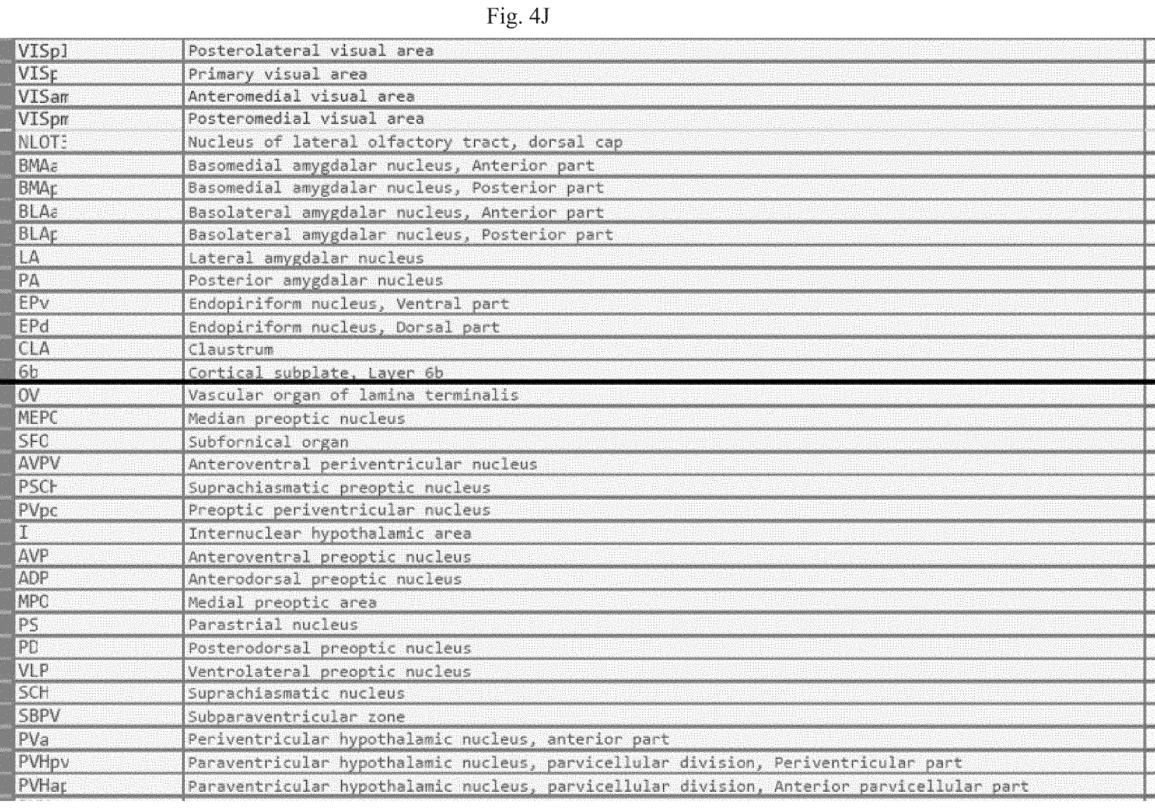

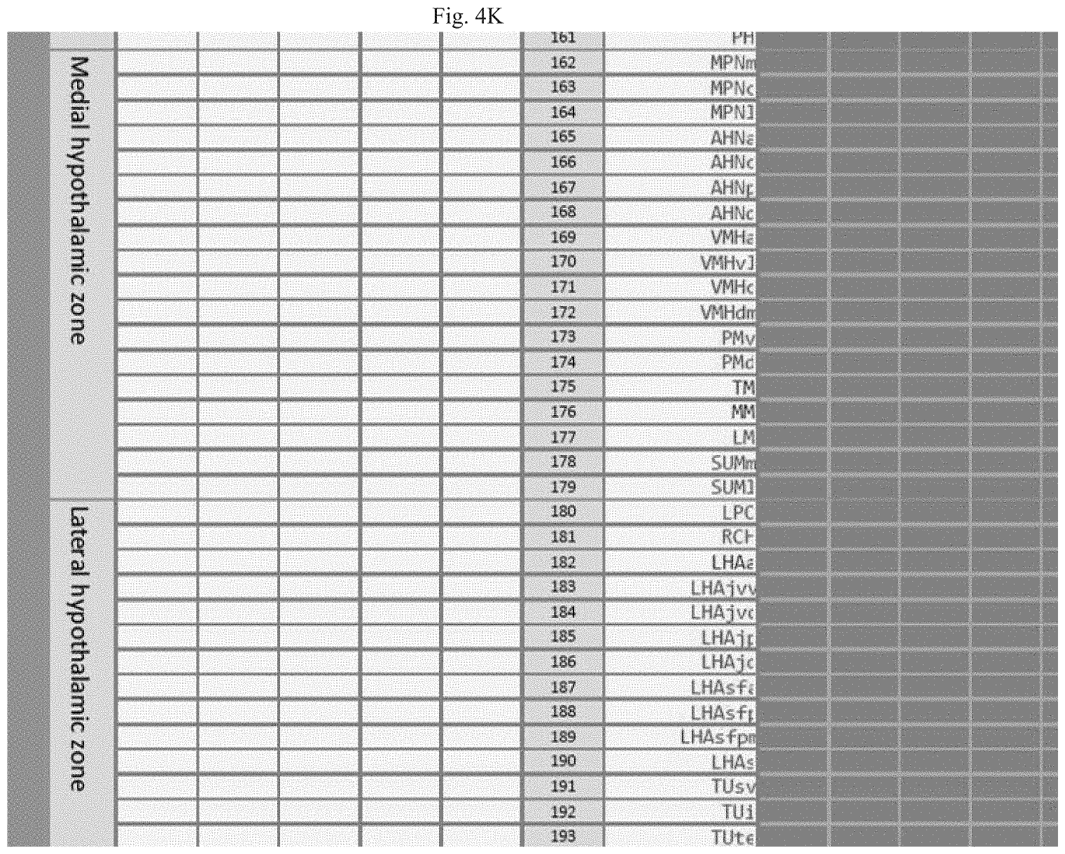

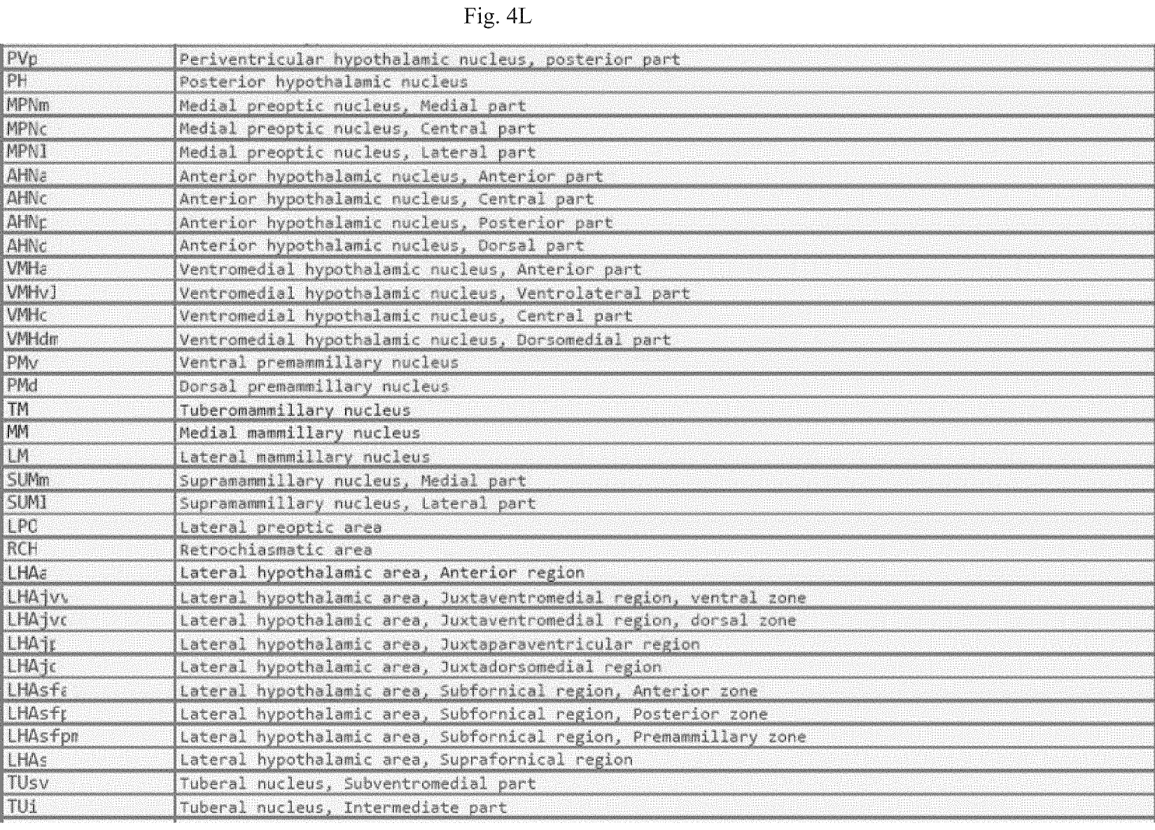

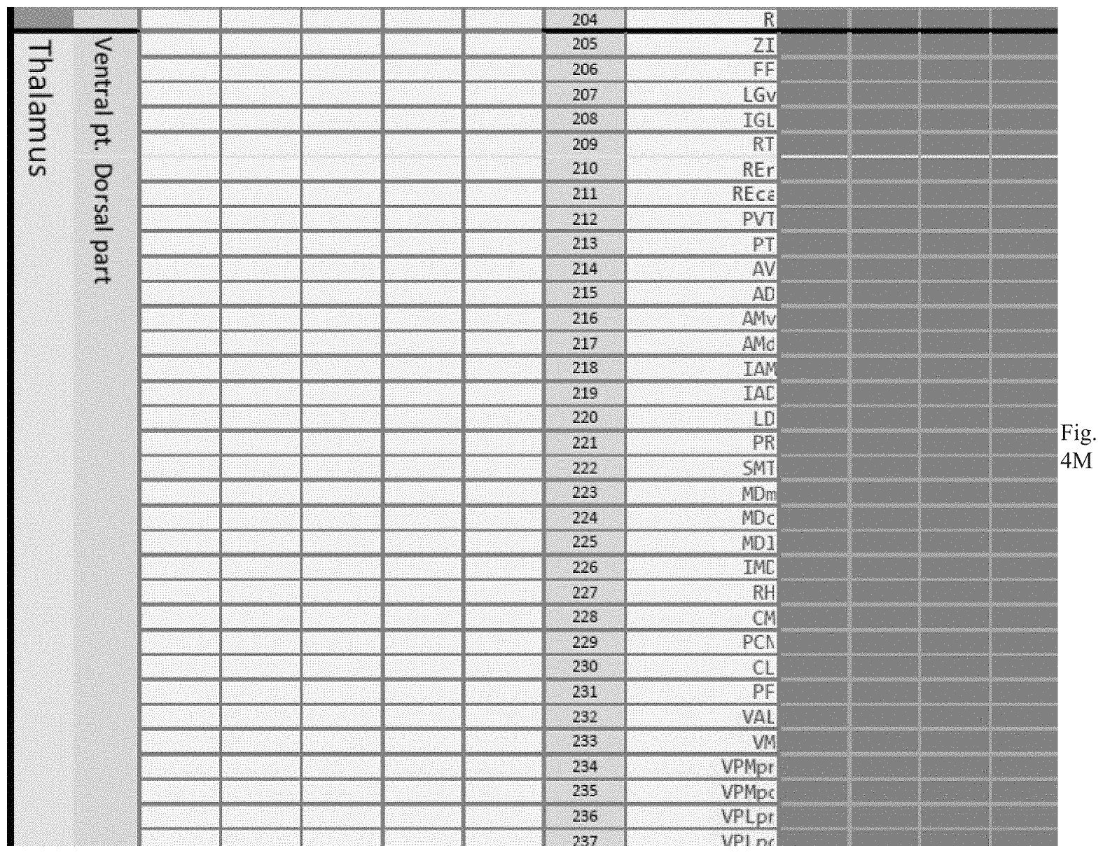

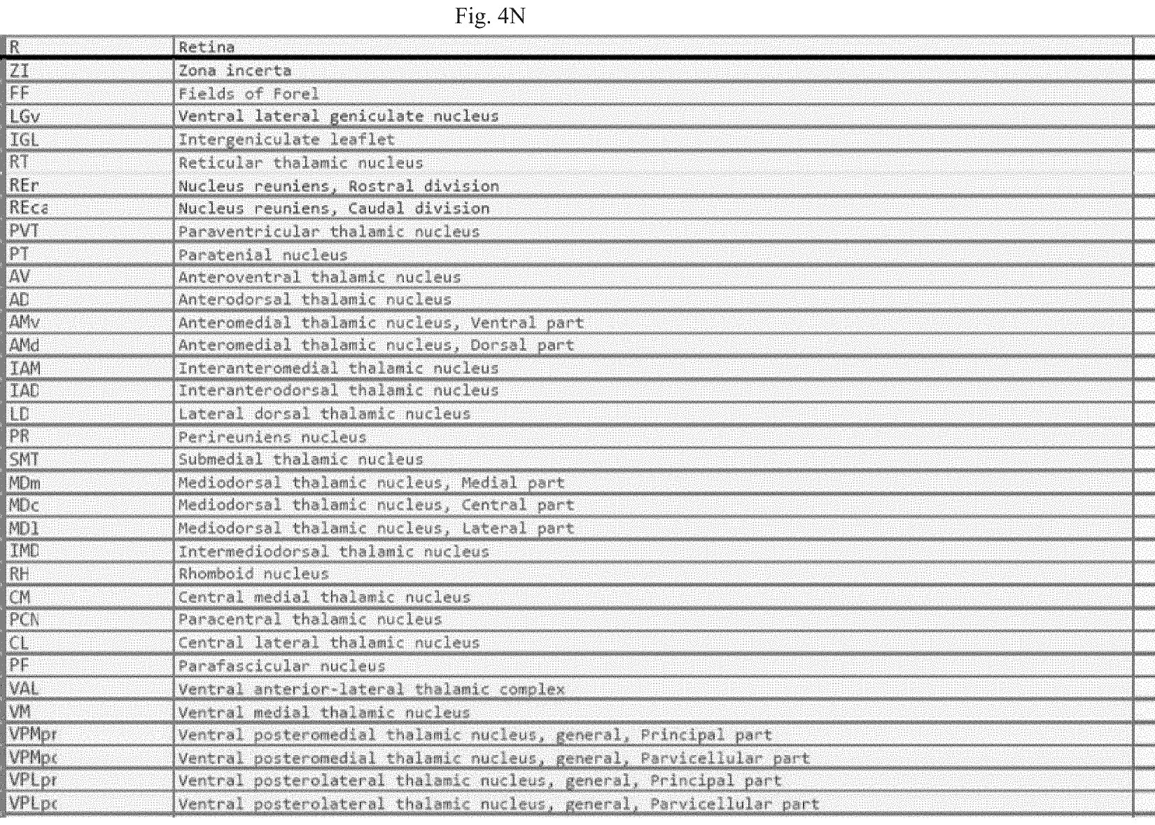

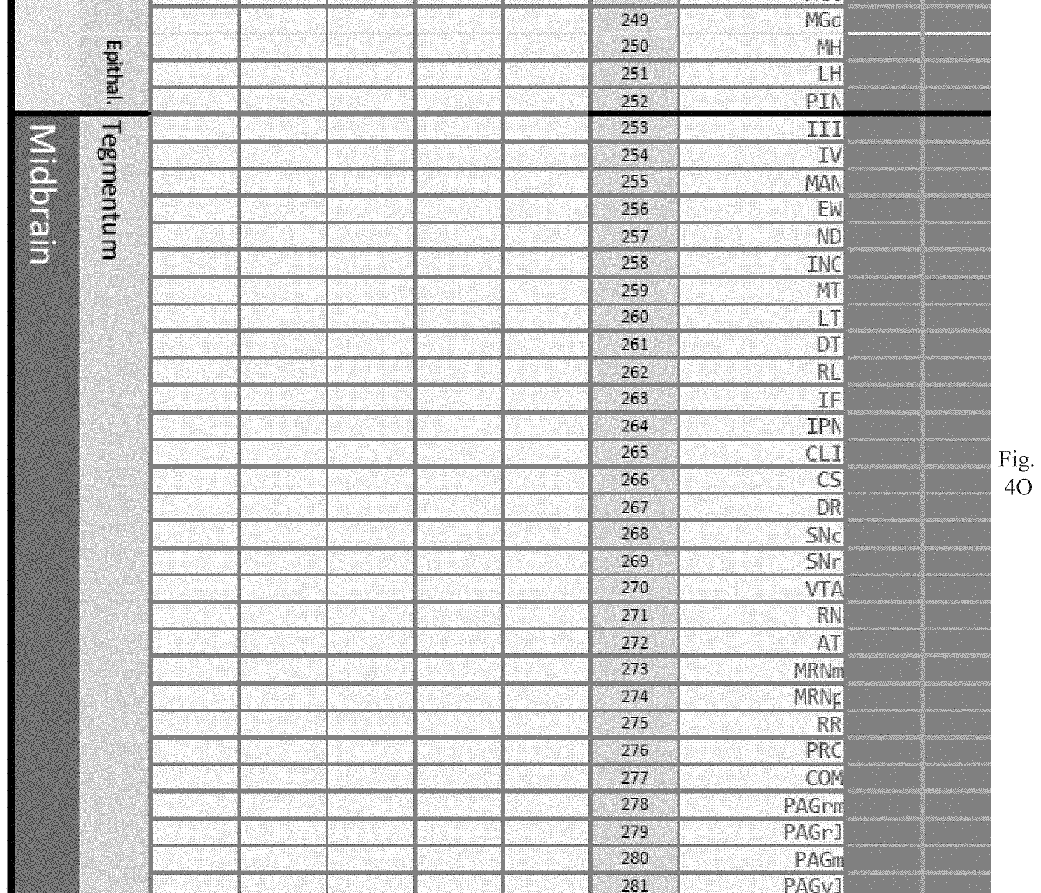

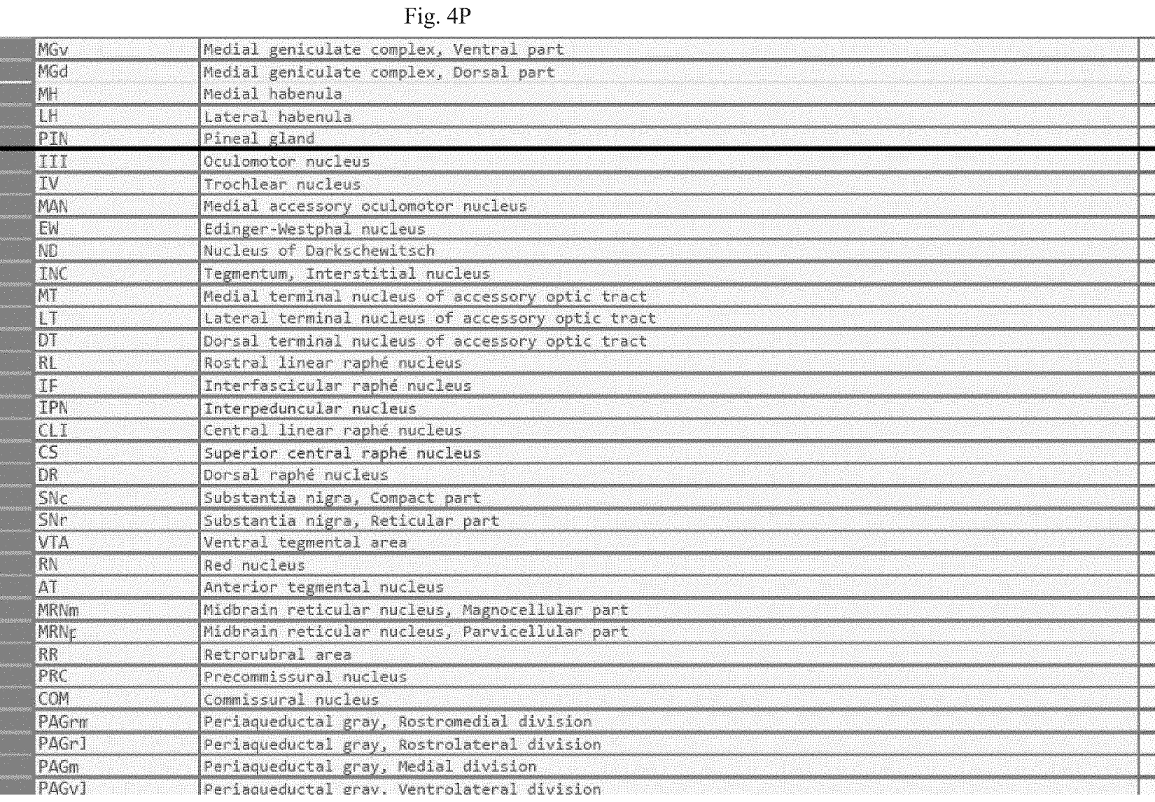

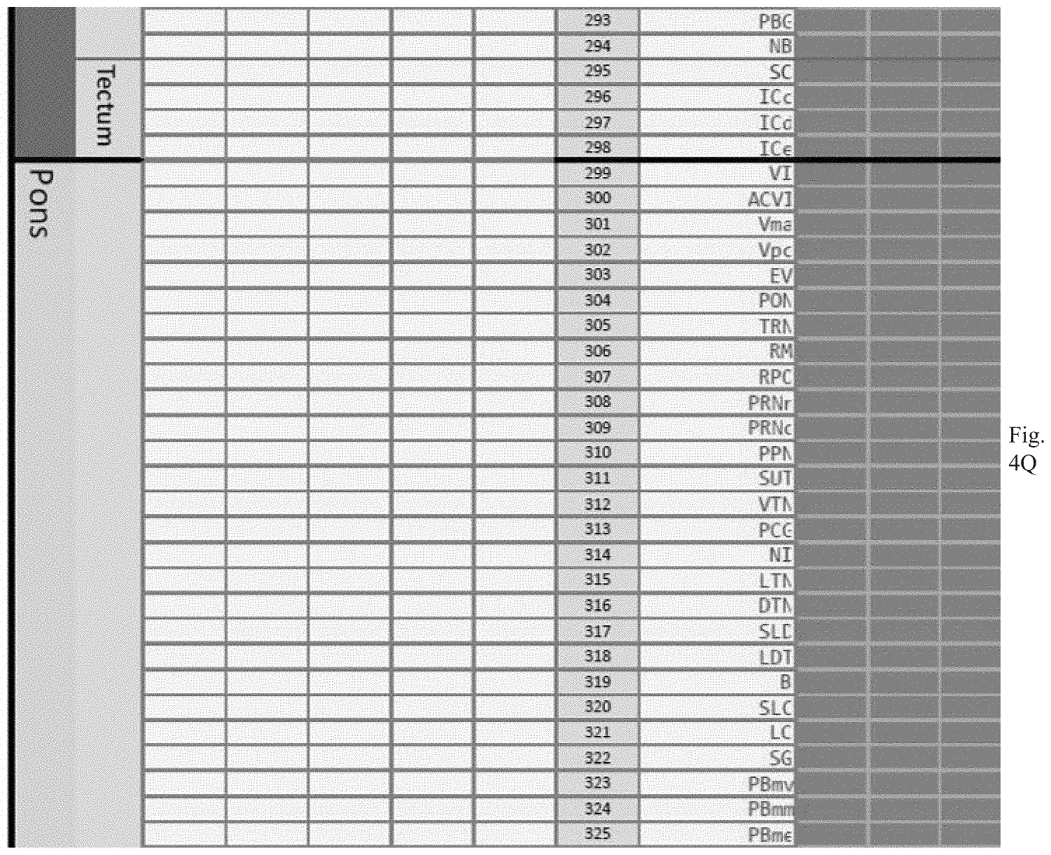

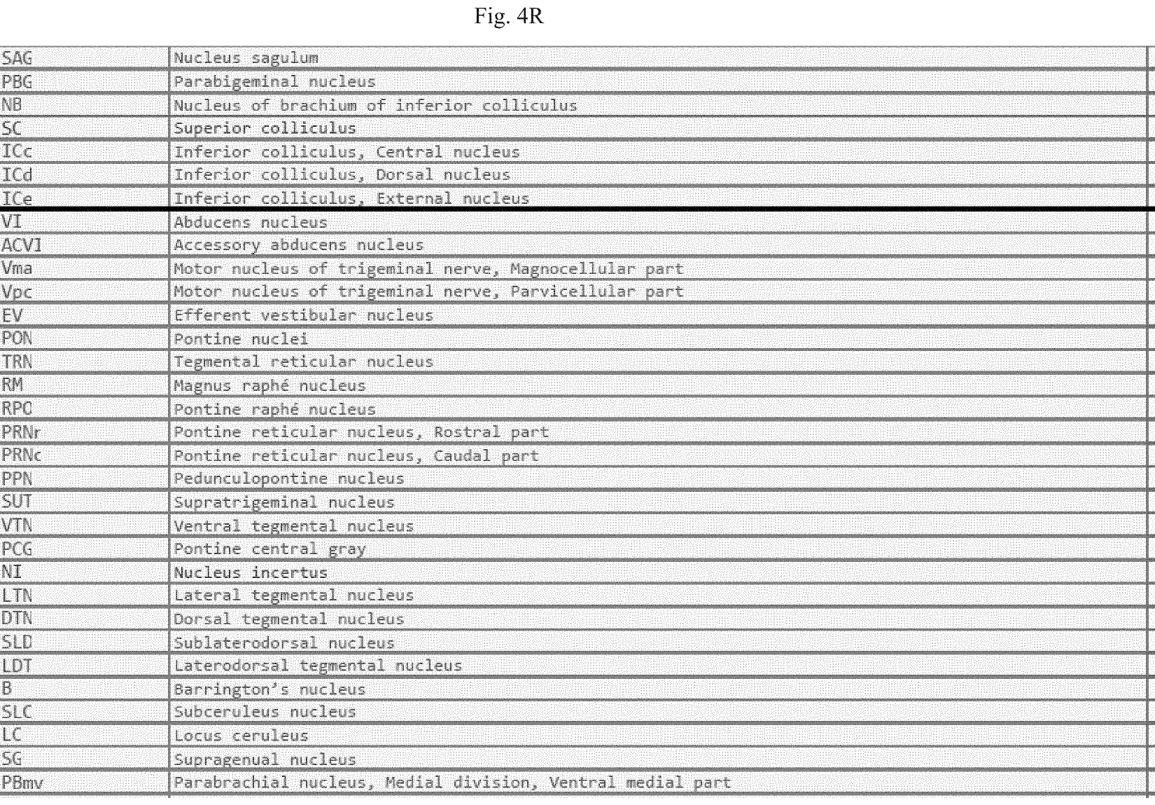

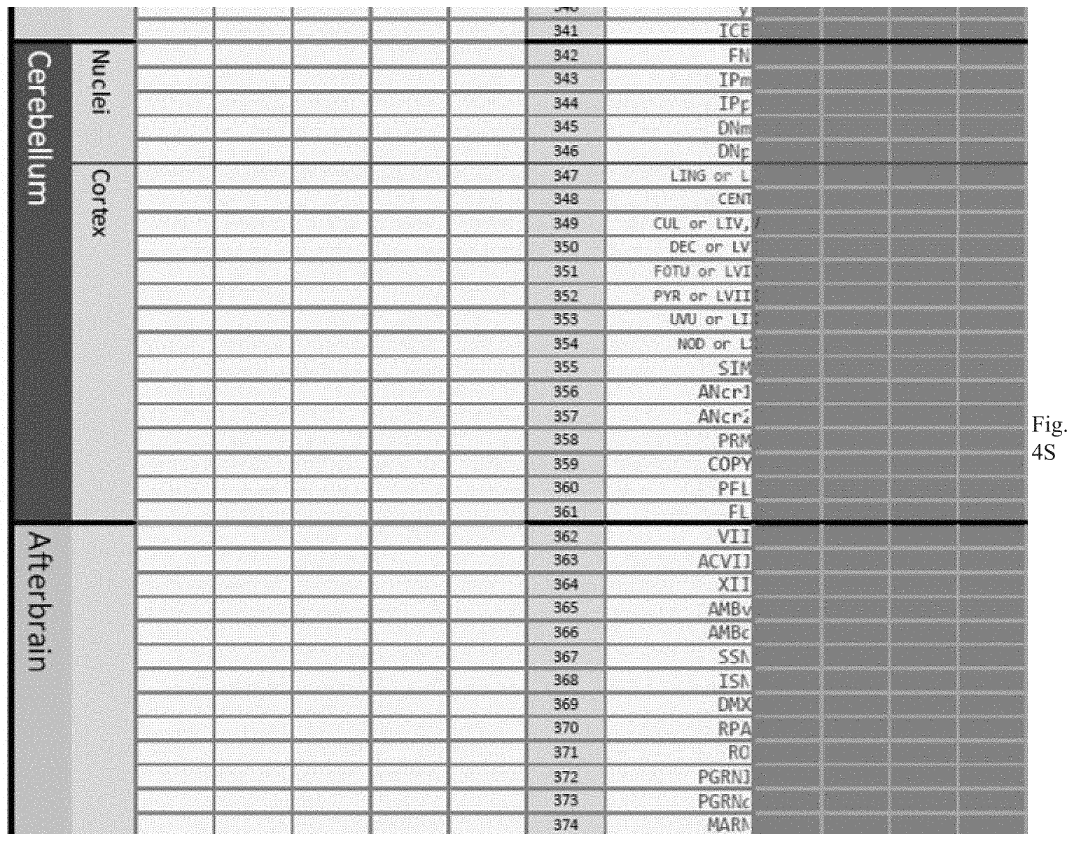

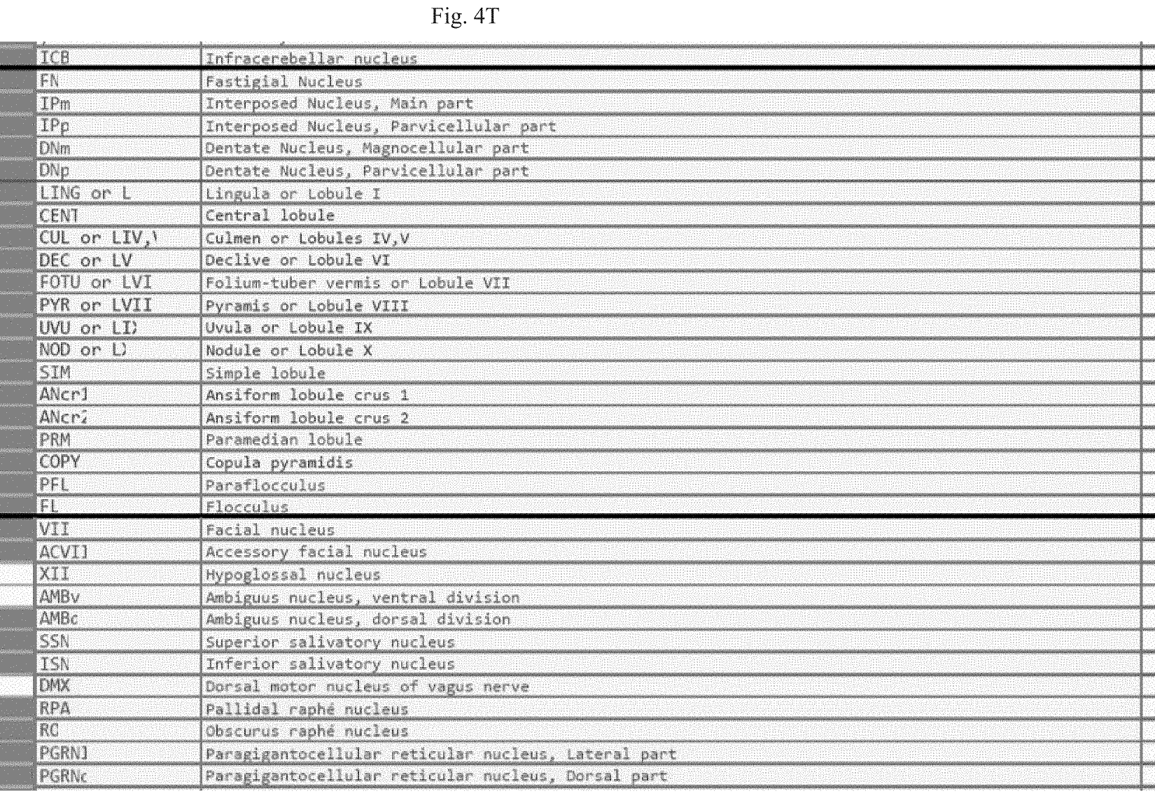

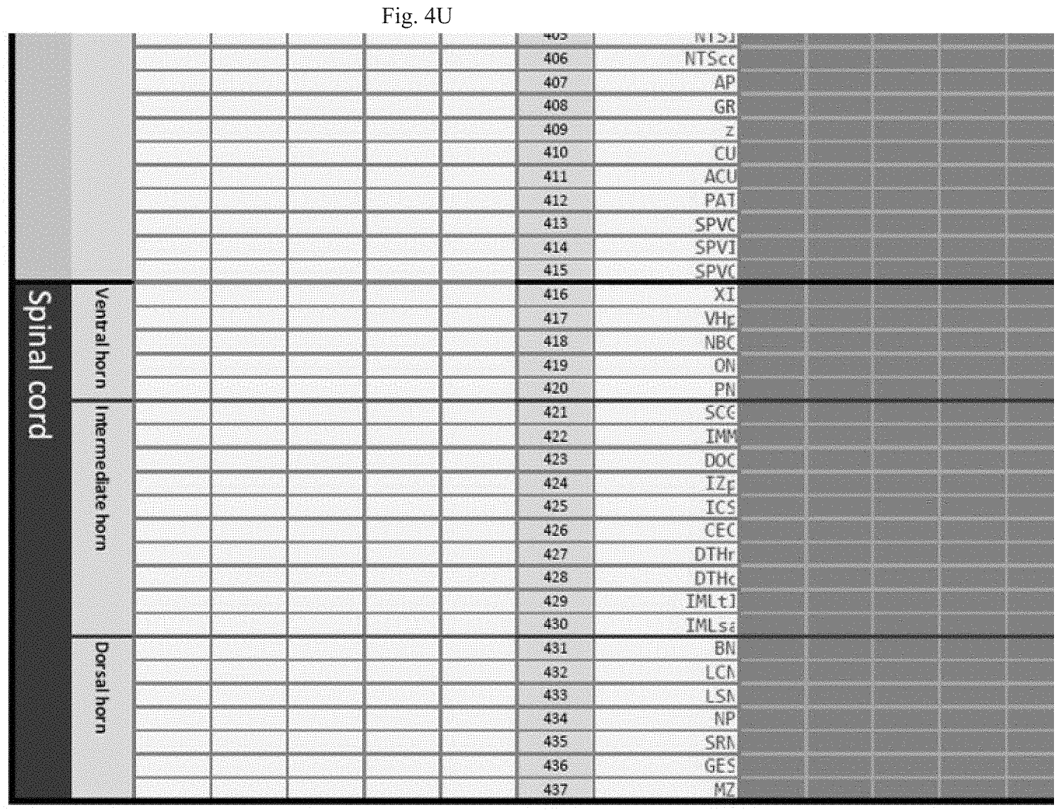

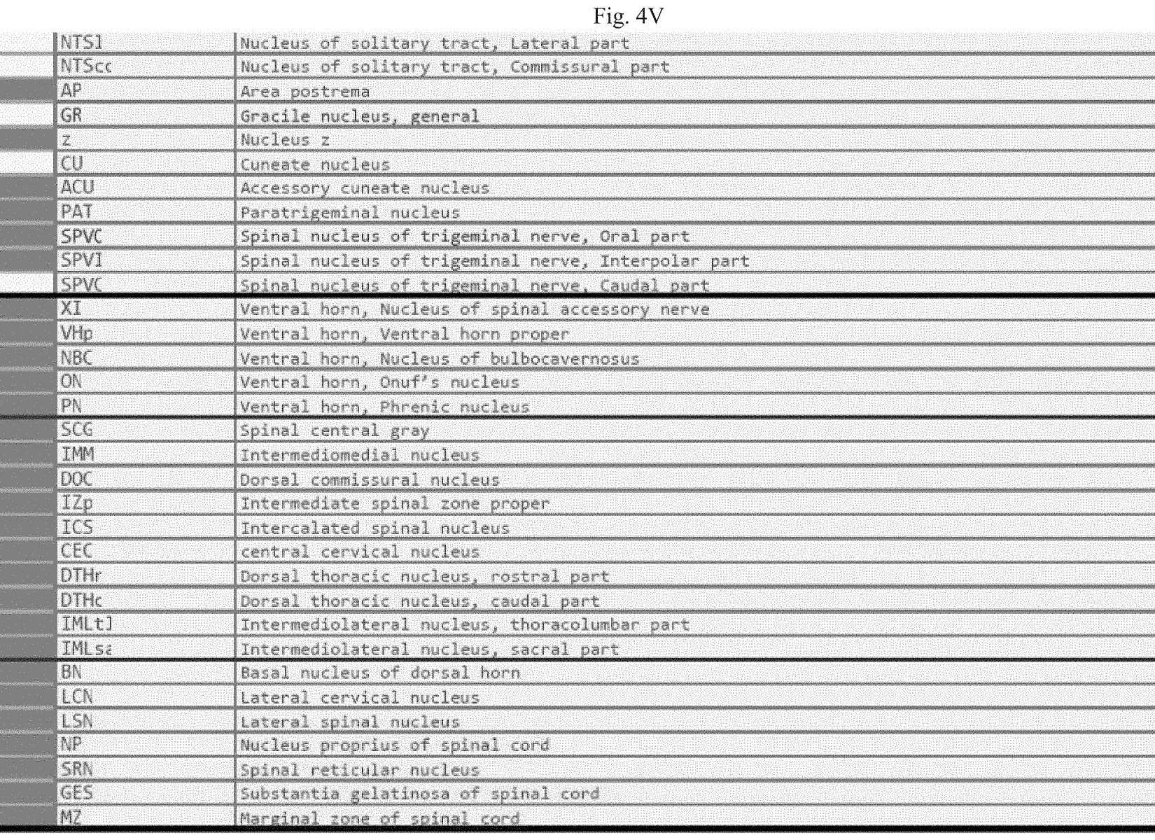

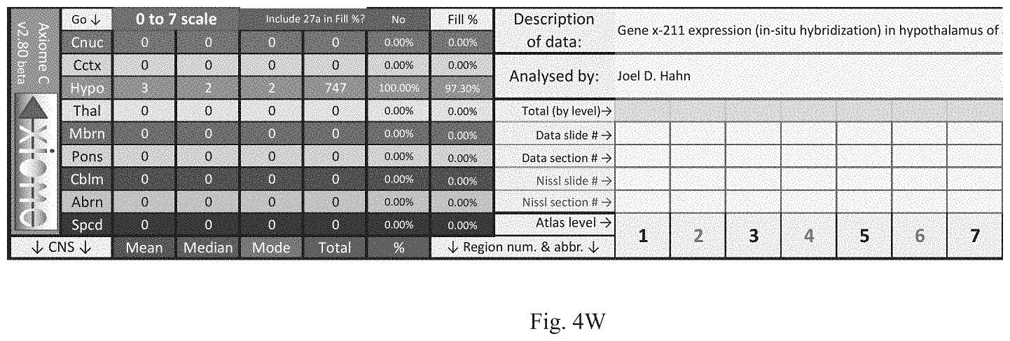

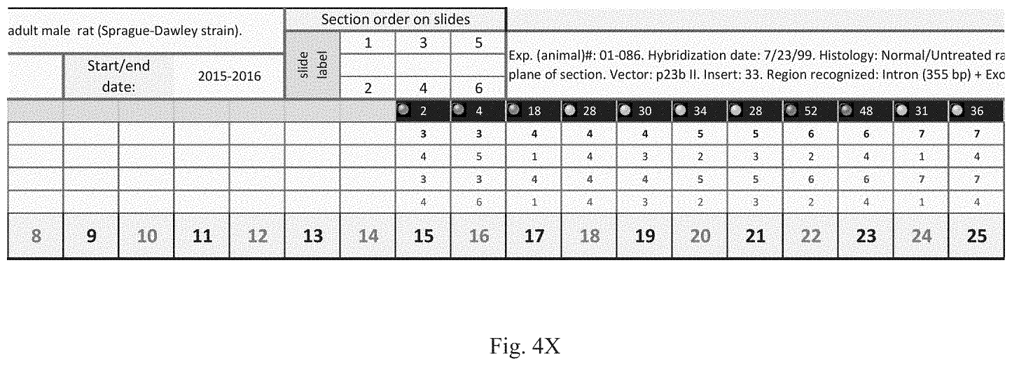

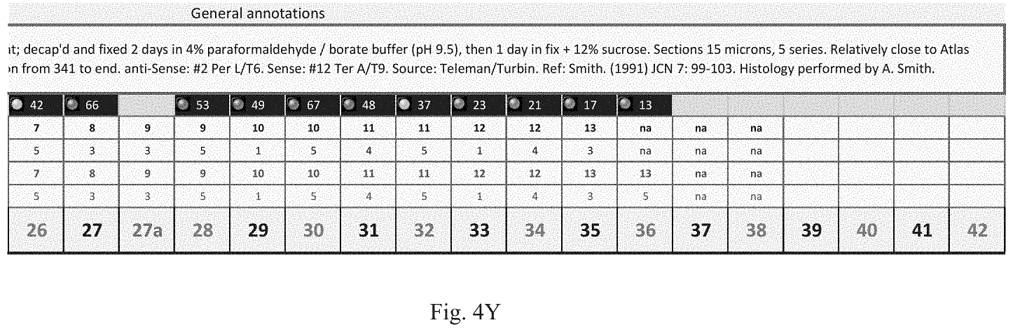

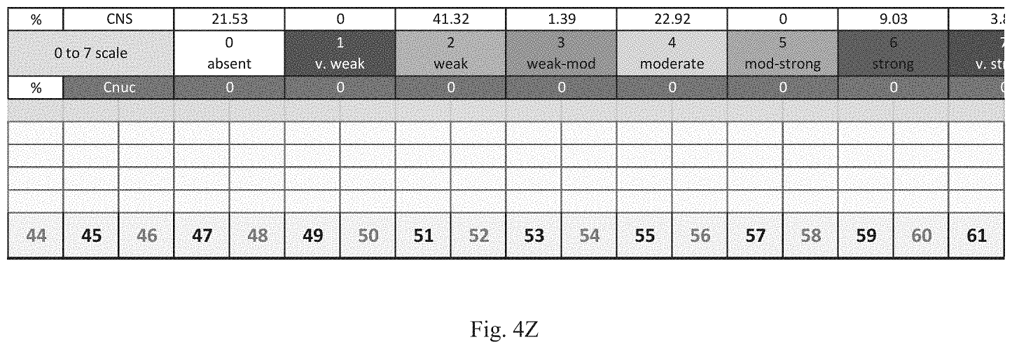

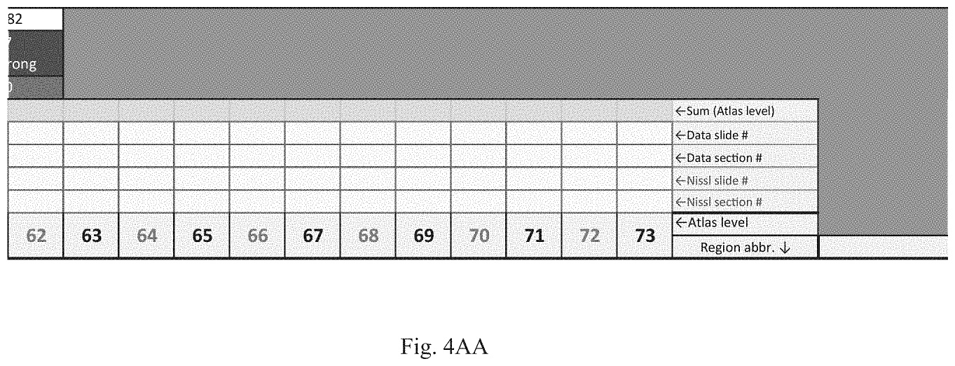

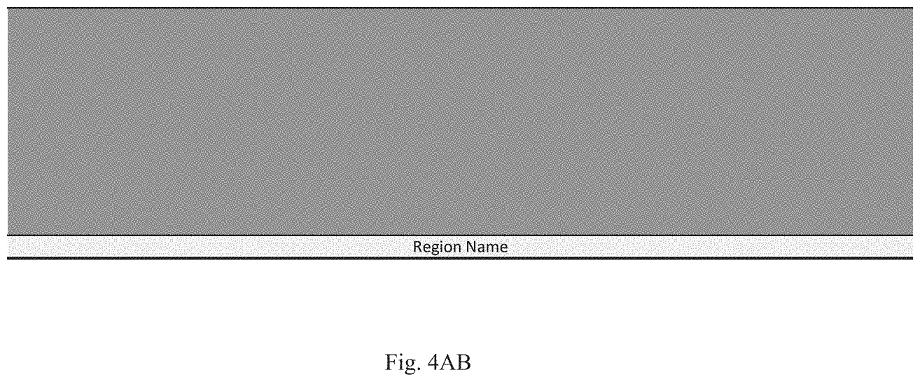

Furthermore, as illustrated in FIGS. 4A-4AB, the Axiome C template includes several exemplary novel features. A key feature is that all gray matter regions of the rat brain for all levels of the aforementioned rat brain atlas are included as data points. This provides a new way to visualize this type of data at high spatial resolution. In addition, descriptive statistics for the data entered are automatically calculated and presented with visual feedback in real-time. The latter facilitates and enables rapid identification of key features and patterns for datasets while the data is in the process of being entered.

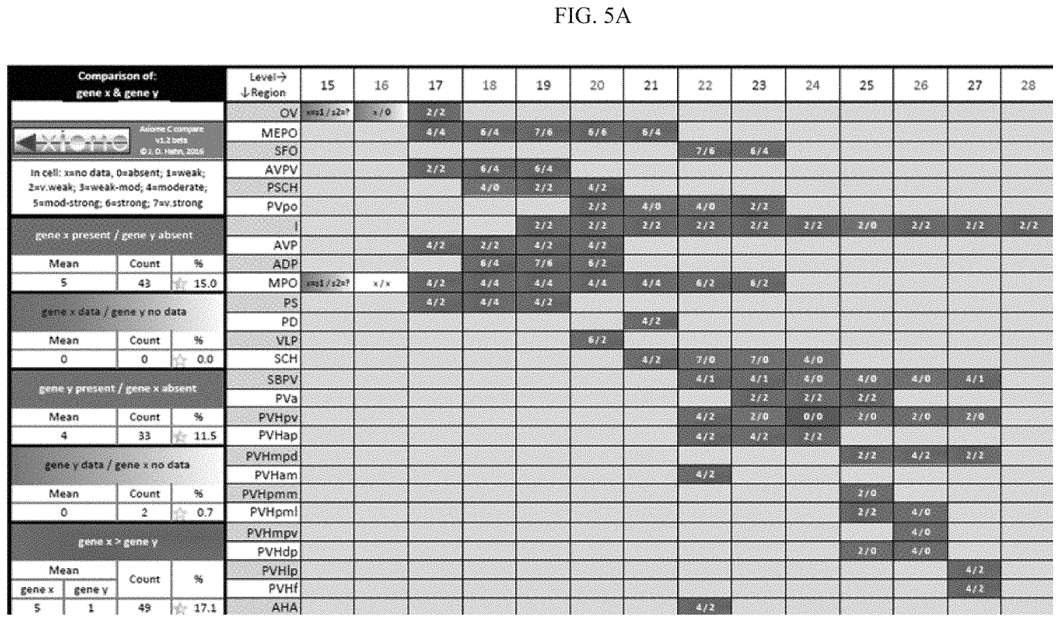

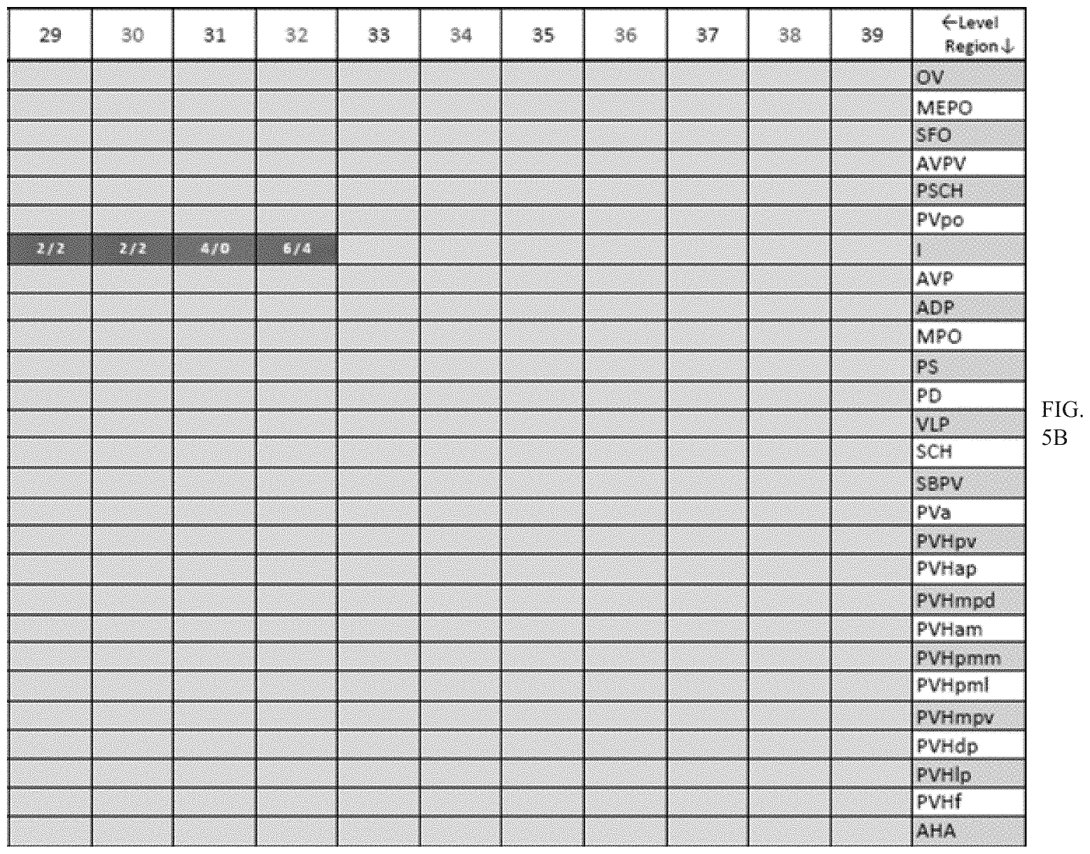

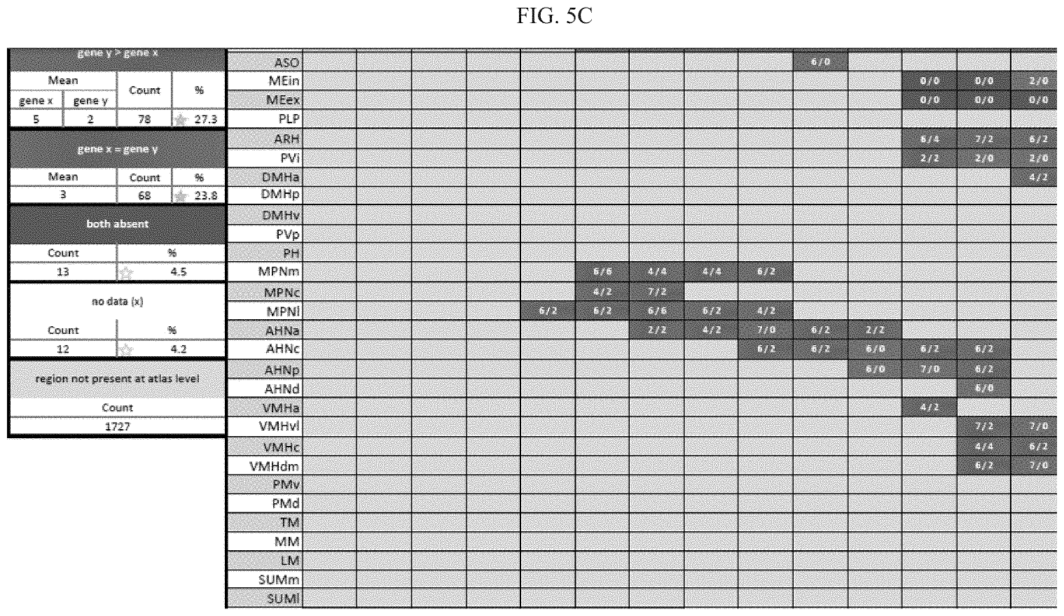

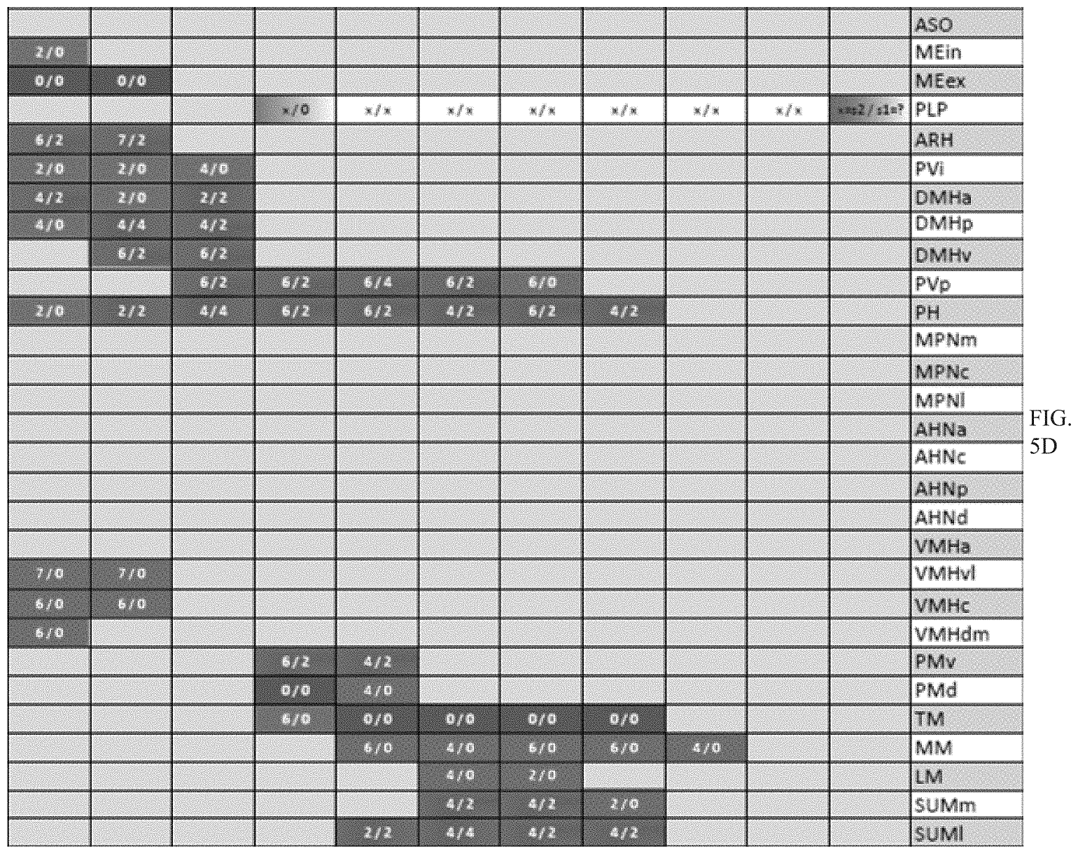

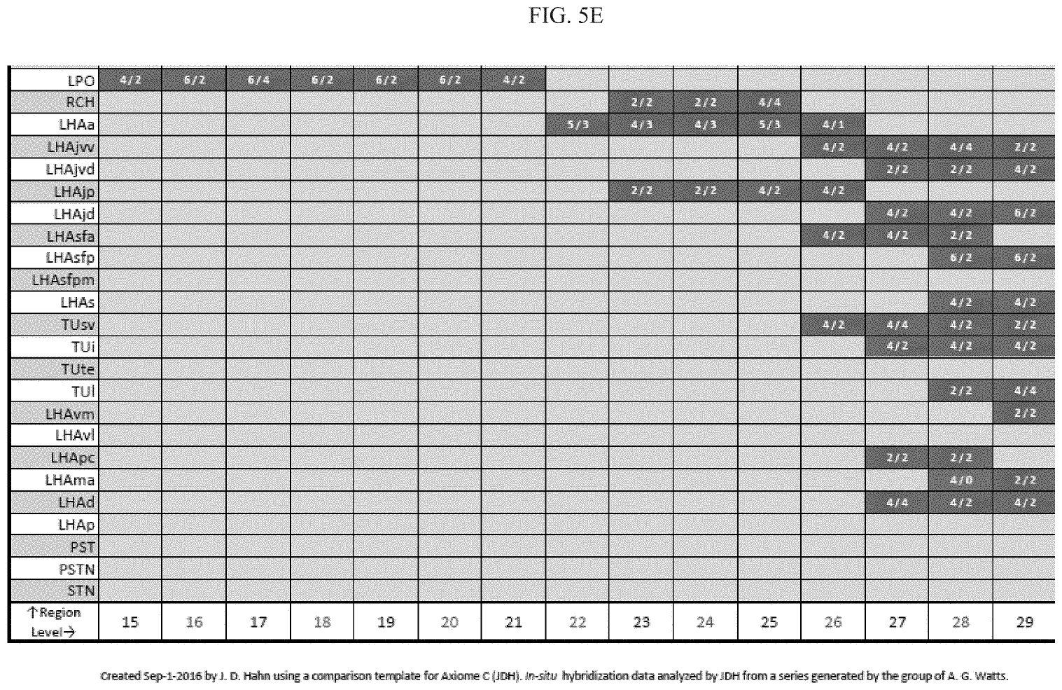

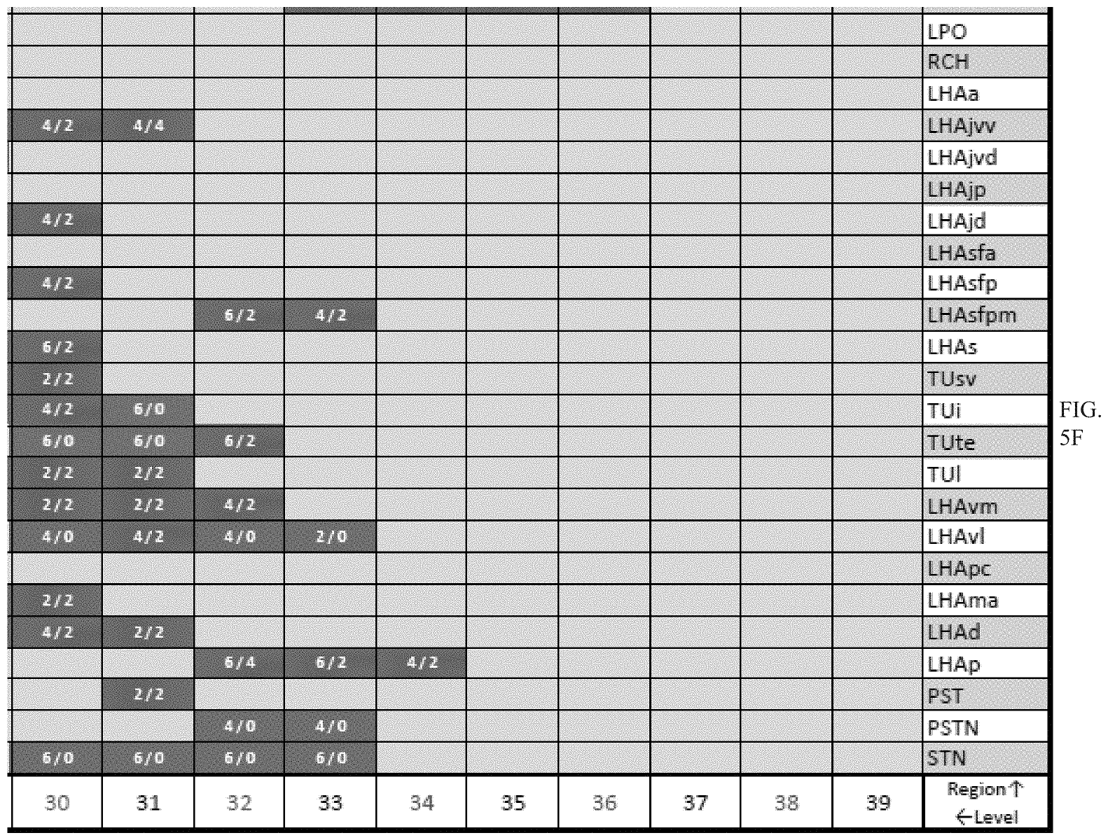

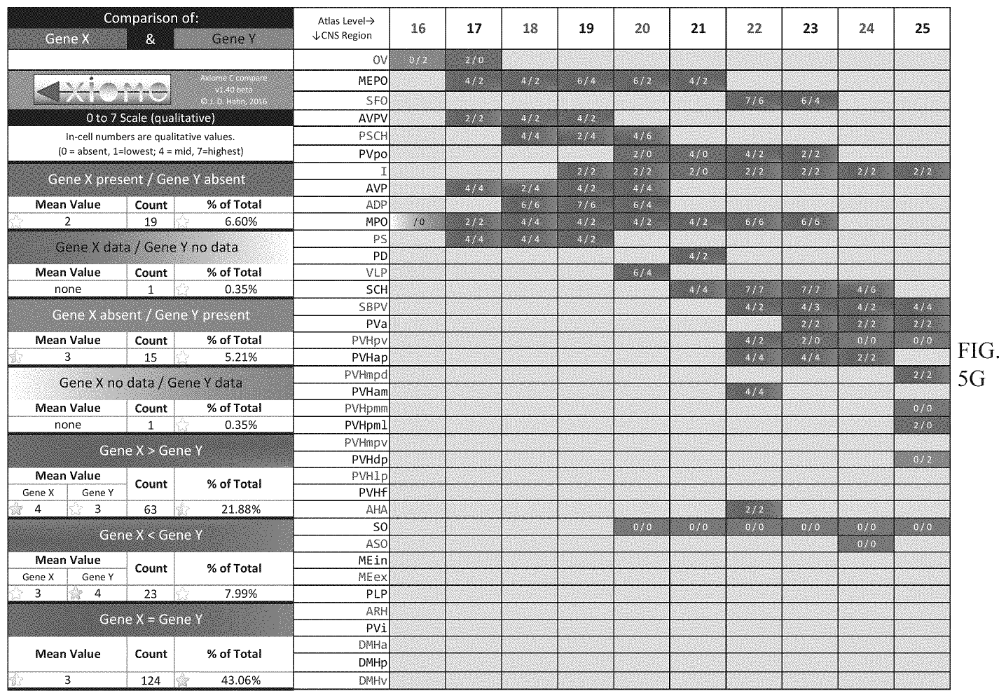

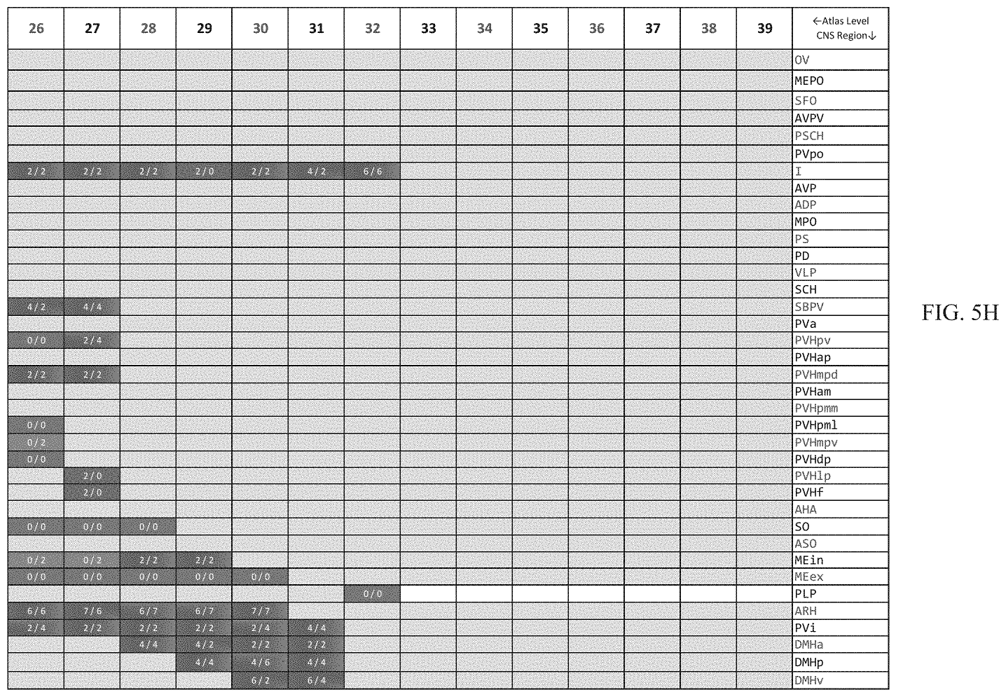

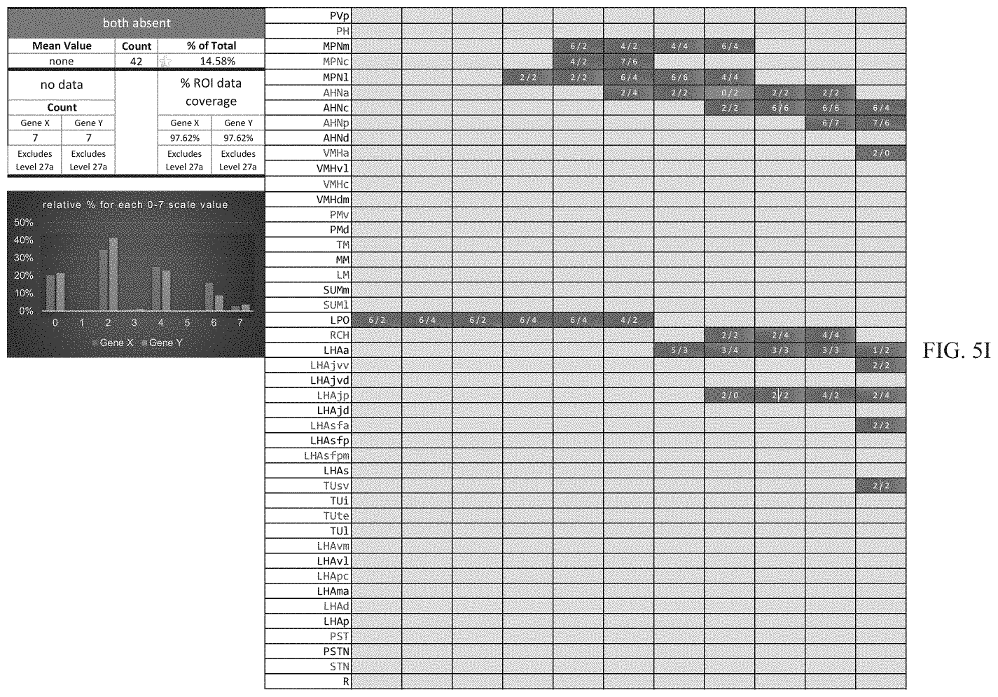

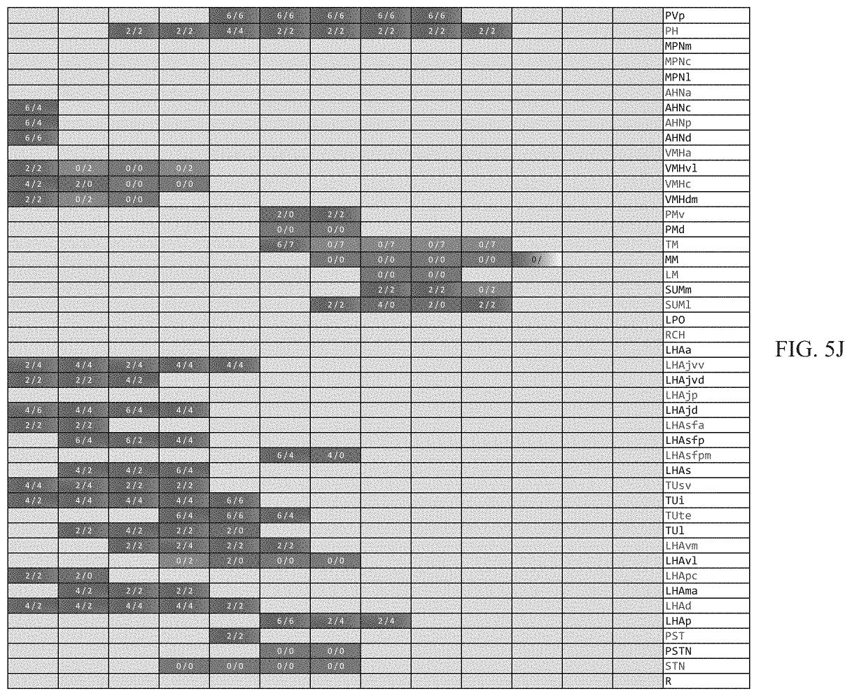

In yet another embodiment, as illustrated in FIGS. 5A-5J, the Axiome tool, C compare template facilitates comparison between any two datasets generated using the Axiome C template. The template uses a series of highly structured formulas and conditional formatting rules to generate a highly visual representation of the compared data, along with detailed descriptive statistics. The combination of graphic visualization and statistics allows for rapid identification of differences and similarities between Axiome C datasets.

In some embodiments, the system and method for modeling connections, structure, and/or function of the nervous system including the CNS and/or PNS, further comprises deriving the data from CNS connection information. In one embodiment, the CNS connection information is data of the cerebral nuclei. In some embodiments, the method further comprises deriving the data from CNS gene expression data including but not limited to expressions of a neurotransmitter, a neurotransmitter receptor, or a cellular or molecular marker such as a marker specific for cells of the CNS. Expression of genes and/or detection of gene products can be assayed by any method known in the art including but not limited to immunohistochemistry, immunofluorescence, flow cytometry, polymerase chain reaction (PCR), quantitative PCR, real-time PCR, gene expression array, mRNA sequencing, high-throughput sequencing, Western blot, Northern blot, and ELISA.

In some embodiments, the system and method for modeling connections, structure, and/or function of the nervous system including the CNS and/or PNS, further comprises presenting the one or more modules as two-dimensional models. In some embodiments, the method further comprises projecting the one or more modules onto the nervous system atlas.

In some embodiments, the ranking of connections comprises one or more of: a node degree, node strength, node betweenness, and node closeness.

In some embodiments, converting the annotated connection matrix includes partitioning the annotated connection matrix into a plurality of modules by modularity maximization.

In one aspect, the method further comprises deriving the data from CNS and/or PNS connection information. In another aspect, the method further comprises deriving the data from nervous system gene expression data, proteomic data, or markers of neural activity. In yet another aspect, the nervous system gene expression data comprises expressions of a neurotransmitter, a neurotransmitter receptor, or a nervous system cellular marker. In still yet another aspect, the method further comprises presenting the one or more modules as two-dimensional models. In yet another aspect, the method further comprises projecting the one or more modules onto an atlas of the nervous system (e.g. an atlas of the rat brain). In another aspect, the ranking of connections comprises one or more of a node degree, node strength, node betweenness and node closeness.

In a yet further embodiment, a method for modeling connections, structure, and/or function of the nervous system including the CNS and/or PNS comprises generating, with a processor, a matrix by use of a nervous system atlas. The method also includes storing the matrix in a first memory location, and receiving data of cerebral nuclei to generate a plurality of connections corresponding to the data of cerebral nuclei, where each of the plurality of connections comprises an origin, a termination, and a degree of connection The method also includes storing the plurality of connections in a second memory location, matching each origin and each termination of the plurality of connections to a corresponding position of the matrix to produce an annotated connection matrix, and converting the annotated connection matrix to one or more modules, where the one or modules comprise an aggregated ranking of connections exceeding a threshold.

While the detailed description herein has focused on the implementation of the present invention utilizing existing software, one of ordinary skill in the art will readily appreciate that the process steps and decisions may be alternatively performed by functionally equivalent software and/or circuits such as a digital signal processor circuit or an application specific integrated circuit (ASIC). Any process flows described above are not intended to describe the exact syntax of any particular programming language, and the flow diagrams illustrate the functional information one of ordinary skill in the art requires to fabricate circuits or to generate computer software to perform the processing required in accordance with the present invention. It should be noted that many routine program elements, such as initialization of loops and variables and the use of temporary variables are not shown. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order.

It is to be understood that embodiments of the invention include the applications (i.e., the un-executed or non-performing logic instructions and/or data) encoded within a computer readable medium such as a floppy disk, hard disk or in an optical medium, or in a memory type system such as in firmware, read only memory (ROM), or, as in this example, as executable code within the memory system (e.g., within random access memory or RAM). It is also to be understood that other embodiments of the invention can provide the applications operating within the processor as the processes. While not shown in this example, those skilled in the art will understand that the computer system may include other processes and/or software and hardware subsystems, such as an operating system, which have been left out for ease of description of the invention.

Furthermore, the data modeling and visualization processes described herein are specifically applied area of cerebral nuclei associations and connections. It will be readily appreciated that the concepts described herein it can be equally applied to any situation that requires large amounts of data to be analyzed for architectures and relationships.

Having described embodiments of the invention it will now become apparent to those of ordinary skill in the art that other embodiments incorporating these concepts may be used. Additionally, the software included as part of the invention may be embodied as computer-readable instructions stored in a computer program product that includes a computer useable medium. For example, such a computer usable medium can include a readable memory device, such as a hard drive device, a CD-ROM, a DVD-ROM, or a computer diskette, having computer readable program code segments stored thereon. The computer readable medium can also include a communications link, either optical, wired, or wireless, having program code segments carried thereon as digital or analog signals. The computer-readable instructions can be executed by one or more processors of a computing device. In addition to a processor and a computer-readable medium such as a memory, the computing device can also include a transceiver, a display, a user interface, and an operating system. Accordingly, it is submitted that that the invention should not be limited to the described embodiments but rather should be limited only by the spirit and scope of the appended claims. Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as hereinafter claimed.

The following examples are provided to illustrate aspects of this invention.

### EXAMPLES

### Example 1

### Cerebral Nuclei Histological Parcellation Granularity

To facilitate comparative analysis of rat connection data, they were collated with reference to a standard rat brain atlas (Swanson L W (2004) A Laboratory Guide with Printed and Electronic Templates for Data, Models and Schematics (Elsevier, Amsterdam), 3^{rd} Ed.), but with gray matter regions of the rat central nervous system (CNS) arranged with respect to a CNS hierarchical nomenclature in a strictly topographic order as outlined elsewhere (Swanson, L. W. (2015) Neuroanatomical Terminology: A Lexicon of Classical Origins and Historical Foundations (Oxford Univ Press, Oxford)), rather than in a structure—function order followed earlier (Bota M, Sporns O, Swanson L W (2015) Proc Natl Acad Sci USA 112(16):E2093-E2101; Swanson L W (2004) A Laboratory Guide with Printed and Electronic Templates for Data, Models and Schematics (Elsevier, Amsterdam), 3^{rd} Ed.; Swanson L W (2003) Oxford Univ Press, Oxford. In addition, the CNS hierarchical levels of gray matter regions and subregions were explicitly recognized as comparable (respectively) to the species and subspecies levels in animal taxonomy (International Commission on Zoological Nomenclature (1999) International Code of Zoological Nomenclature (International Trust for Zoological Nomenclature, London) (Table 4). This nomenclature scheme recognizes 45 gray matter regions in the rat cerebral nuclei, all of which are included in the present analysis.

### Connection Report Collation and Selection for Network Analysis

First, the primary literature was searched to find the best available connection data, from which connection reports were created. Several criteria were used to assess the quality of connection data: these included the validity of the experimental pathway tracing method used, restriction of the pathway tracer injection site to the gray matter region of interest, injection site coverage of the region of interest, and thoroughness of description of the connection. Next, for each possible connection in the connectome for which data were available, a single connection report was selected as best representative of the connection (using the criteria noted above). If more than one connection report was created for a given connection (depending on the availability of data in the primary literature), then, all else being equal, the connection report with the highest connection weight was selected. Finally, the weight of each selected connection report was used to populate a connection matrix that was used for subsequent network analysis.

The process of collation was considerably aided by the use of a Q:1 dedicated data entry platform designed as a spreadsheet template for use with Microsoft Excel or another similar program: Axiome M module. The template facilitates speed and accuracy of data entry by using data validation and conditional formatting rules and a highly structured and guided user-friendly interface.

### Connection Weight Scaling Methodology for Network Analysis

There are almost no quantitative data available in the literature for the rat macroconnections used in this analysis. Therefore, ranked qualitative connection weights from the literature were divided into 12 value categories. In ascending order, they are no data, unclear, absent, axons of passage, very weak, weak, weak to moderate, present (value unreported), moderate, moderate to strong, strong, and very strong. For the purposes of a network analysis, reports of axons of passage were assigned a weight of “weak,” and connections for which the reported value was entered as “present” (weight unreported) were assigned a weight of “moderate.” When network analysis was applied to the dataset, Q:2 the category values of unclear and no data were assigned to the absent category. Thus, the set of ranked qualitative values used for network analysis included 8 values (7 weights and 0 for absent that were considered for our purposes to form an ordinal scale. As justified previously (Bota M, Sporns O, Swanson L W (2015) Proc Natl Acad Sci USA 112(16):E2093-E2101), the ranked qualitative connection weights were then transformed to approximately logarithmically spaced weights for network analysis, using a 10⁴ exponential scale.

### Network Analysis Methods

Network analyses were carried out on the directed and log-weighted rat intracerebral nuclei macroconnection (RiCNM) matrix (FIGS. 6, 9, and 19), using tools collected in the Brain Connectivity Toolbox (www.brainconnectivity-toolbox.net). Detailed descriptions of most network measures can be found in Rubinov, M. (2010) Neuroimage 52(3): 1059-69. Rat cerebral nuclei gray matter regions are referred to as nodes of the RiCNM network.

For detection of optimal module partitions the Louvain algorithm (Blondel V, Guillaume J L, Lambiotte R, Lefebvre E (2008). J Stat Mech P10008) was implemented for modularity maximization (Sprons O. Betzel R F (2016) Annu Rev Psychol 67:613-640; Newman M E J, Girvan M (2004) Phys Rev E Stat Nonlin Soft Matter Phys 69(2 pt 2):026113), including a resolution parameter γ designed to address a known limitation of modularity optimization, the resolution limit (Fortunato S, Barthelemy M (2007) Proc Natl Acad Sci USA 104(1):36-41). Varying γ effectively allows the detection of modules that range over several spatial scales. In one study, the parameter γ was varied over a range of γ=[0.5-1.5], an interval centered on the default setting of γ=1; higher and lower settings of γ yielded mostly unstable solutions with unrealistically low or high numbers of modules. Robust module partitions are expected to remain stable over a broad range of settings of γ; that is, they should be insensitive to small variations in spatial scale. The modularity was optimized 1,000 times for each setting of γ and encountered very little degeneracy in the distribution of solutions across these 1,000 iterations. Hence a selection was made of the globally optimal module partition at each level of γ for further analysis.

Analyses of global network metrics such as clustering and efficiency, reciprocity, and rich club organization were statistically evaluated by comparison with a degree-sequence—preserving distribution of null models, as in previous work (Bota M, Sporns O, Swanson L W (2015) Proc Natl Acad Sci USA 112(16):E2093-E2101). Rewiring of the networks composing the random null model followed a commonly used procedure equivalent to a Markov switching algorithm (Maslov S, Sneppen K (2002) Science 296(5569):910-913) that preserves the number of incoming and outgoing connections on all nodes.

As in previous work (Bota M, Sporns O, Swanson L W (2015) Proc Natl Acad Sci USA 112(16):E2093-E2101; Sporns O, Honey C J, Kötter R (2007) PLoS One 2(10):e1049; Harriger L, van den Heuvel M P, Sporns O (2012) PLoS One 7(9):e46497), network hubs were determined on the basis of aggregated rankings across several distinct nodal centrality measures. These measures were node degree, node strength, node betweenness centrality, and closeness centrality. The node degree is defined as the sum of all incoming and outgoing connections per node. The node strength is defined as the total weight of all incoming and outgoing connections per node (computed from the weighted connection matrix). The node betweenness expresses the fraction of shortest paths that pass through each node. Closeness was calculated as the average of the row an column sum of the network's distance matrix. Betweenness and closeness were both derived from the weighted connection matrix, after converting connection weights to lengths, using an inverse transform. After ranking nodes on each of the four metrics, an aggregate “hub score” was determined for each node, expressing the number of metrics for which each node appeared in the top 20% (top nine nodes).

Rich club organization refers to a simple property that is shared by many, but not all, complex biological networks (Colizza V, Flammini A, Serrano M A, Vespignani A (2006) Nat Phys 2:110-115)—the propensity of highly connected nodes (that is, nodes with high degree) to also be densely connected to each other, more so than expected by chance. First, for each value of node degree k, the total sum of the weights W>k between all nodes with degree k or higher was determined. No distinction is made between incoming and outgoing connection weights. Next, the weighted rich club coefficient Φw(k) was computed as the ratio between W>k and the sum of the weights of the strongest E>k connections across the whole network. The weighted rich club coefficient was then normalized against a set of 10,000 randomly rewired networks, preserving network size, density, and degree sequence (see above). Comparison of the rich club coefficient of the empirical network to this random null distribution was then subjected to significance testing. To correct for multiple comparisons over the range of degrees k examined, false-discovery rate correction was performed (Benjamini, Y. et al. (1995) J R Stat Soc B 57:289-300), at a false discovery rate of 0.001.

Reciprocity was assessed following the approach by Squartini et al. (Squartini, T. et al. (2013) Sci Rep. 3:2729). Briefly, the network was decomposed into a symmetric (reciprocated) part and an asymmetric (nonreciprocated) part. The weighted reciprocity of the network was then computed as the ratio between the total reciprocated weight (the sum of all of the weights contained in the reciprocated part) and the total weight of the network. This quantity was then scaled relative to the average weighted reciprocity derived from the degree-sequence—preserving random null model (see above). The resulting metric ρ indicates the tendency of the network to reciprocate (ρ>0) or to avoid reciprocation (ρ<0). In the case of ρ>0, a higher value of ρ indicates a stronger tendency to reciprocate.

Additional non-limiting embodiments of the present disclosure are described in Example 2.

### Example 2. Network Architecture of the Cerebral Nuclei (Basal Ganglia) Association and Commissural Connectome

The cerebral nuclei form the ventral division of the cerebral hemisphere and are thought to play an important role in neural systems controlling somatic movement and motivation. Network analysis was used to define global architectural features of intrinsic cerebral nuclei circuitry in one hemisphere (association connections) and between hemispheres (commissural connections). The analysis was based on more than 4,000 reports of histologically defined axonal connections involving all 45 gray matter regions of the rat cerebral nuclei and revealed the existence of four asymmetrically interconnected modules. The modules form four topographically distinct longitudinal columns that only partly correspond to previous interpretations of cerebral nuclei structure—function organization. The network of connections within and between modules in one hemisphere or the other is quite dense (about 40% of all possible connections), whereas the network of connections between hemispheres is weak and sparse (only about 5% of all possible connections). Particularly highly interconnected regions (rich club and hubs within it) form a topologically continuous band extending through two of the modules. Connection path lengths among numerous pairs of regions, and among some of the network's modules, are relatively long, thus accounting for low global efficiency in network communication. These results provide a starting point for reexamining the connectional organization of the cerebral hemispheres as a whole (right and left cerebral cortex and cerebral nuclei together) and their relation to the rest of the nervous system.

### Significance

The cerebral nuclei together with the cerebral cortex form the cerebral hemispheres that are critically important for the control of voluntary behavior and motivation. Network analysis of microscopic connectional data collected since the 1970s in a small, intensely studied mammal provides a new way to understand overall design features of circuitry coordinating activity in the various parts of the cerebral nuclei on both sides of the brain. Basically, intracerebral nuclei circuitry is organized into four modules on each side of the brain, with connections within and between modules on one side being quite dense and connections between the cerebral nuclei on either side being quite sparse. The results provide a perspective on cerebral nuclei structure and function.

The paired adult vertebrate cerebral hemispheres are differentiations of the embryonic neural tube's endbrain (telencephalic) vesicle, which in turn forms a ventral nonlaminated part, the cerebral nuclei, and then a dorsal laminated part, the cerebral cortex (Herrick, C. J. (1910) J Comp Neurol Psychol 20(5):413-547; Alvarez-Bolado, G. et al. (1996) Developmental Brain Maps: Structure of the Embryonic Rat Brain (Elsevier, Amsterdam); Nieuwenhuys, R. et al. (2008) The Human Central Nervous System (Springer, Berlin), 4th Ed.). In mammals the largest parts of the cerebral nuclei by volume are the caudoputamen (striatal) and globus pallidus (pallidal). Together, they are commonly regarded as the endbrain parts of the basal ganglia (Nieuwenhuys, R. et al. (2008) The Human Central Nervous System (Springer, Berlin), 4th Ed.), which play an important role in controlling skeletomuscular (somatic) movements and in the etiology of movement disorders (DeLong, M. R. et al. (2015) JAMA Neurol. 72(11):1354-1360).

A major shift in thinking about the basal ganglia occurred with the recognition that certain ventral parts of the cerebral nuclei display the same basic circuit organization, but involve different parts of a cerebral cortex to cerebral nuclei to thalamus to cerebral cortex loop (Heimer, L. et al. (1975) Golgi Centennial Symposium Proceedings, ed Santini M (Raven, New York):177-193). This finding led to an expanded view of the basal ganglia, with dorsal and ventral striatopallidal subsystems involved primarily in movement (dorsal) and motivational functionality (ventral) (de Olmos, J. S. et al. (1999) Ann NY Acad Sci. 877:1-32). Complete expansion of the view was then proposed on the basis of connectional, gene expression, embryological, and functional evidence (Swanson, L. W. (2000) Brain Res. 886(1-2):113-164). It was hypothesized that the entire cerebral cortex, cerebral nuclei, and thalamus can be divided into four basic subsystems involving dorsal, ventral, medial, and caudorostral domains of the striatopallidum.

This example reexamines the organization of axonal connections between all parts of the cerebral nuclei, using systematic, data-driven, network analysis methods. The analysis uses a directed, weighted macroconnectome of association connections (between ipsilateral parts) and commissural connections (between parts on one side and those on the other side), with novel analytical approaches and curation tools. In this approach a macroconnection is defined as a monosynaptic axonal (directed, from/to) connection between two nervous system gray matter regions or between a gray matter region and another part of the body (such as a muscle) (Swanson, L. W. et al. (2010) Proc Natl Acad Sci USA 107(48):20610-20617; Brown, R. A. et al. (2013) J Comp Neurol 521(13):2889-2906). All 45 gray matter regions of the cerebral nuclei on each side of the brain were included in the analysis. The goal of this analysis was to provide global, high-level, design principles of intrinsic cerebral nuclei circuitry as a framework for more detailed research at the meso-, micro-, and nanolevels of analysis.

### Results

Systematic curation of the primary neuroanatomical literature yielded no reports in the literature of statistically significant male/female, right/left, or strain differences for any association or commissural connection used in the analysis, which therefore simply applies to the adult rat in the absence of further data. The entire dataset was derived from 4,067 connection reports expertly curated from 40 peer-reviewed original research articles; 2,731 or 67.2% of the connection reports were from the L. W. S. laboratory. A standard rat brain atlas nomenclature (Table 4) was used to describe all connection reports, which were based on a variety of experimental monosynaptic anterograde and retrograde axonal pathway tracing methods identified for each connection report.

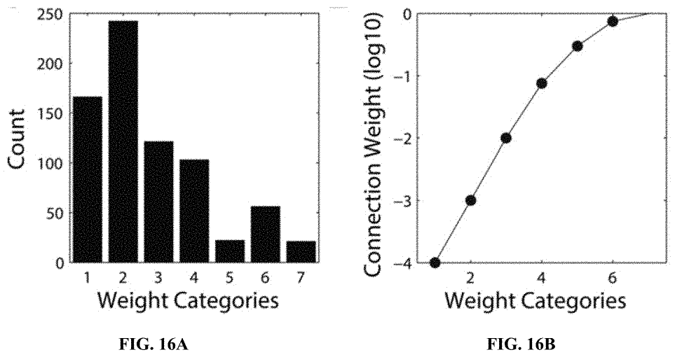

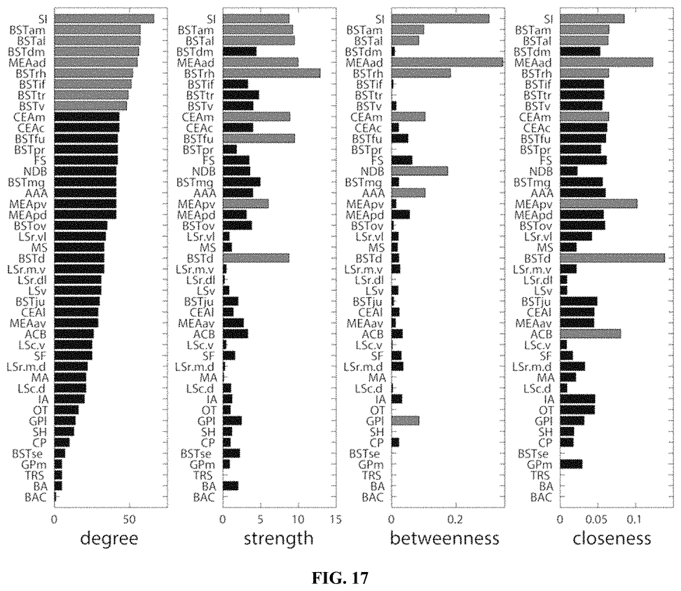

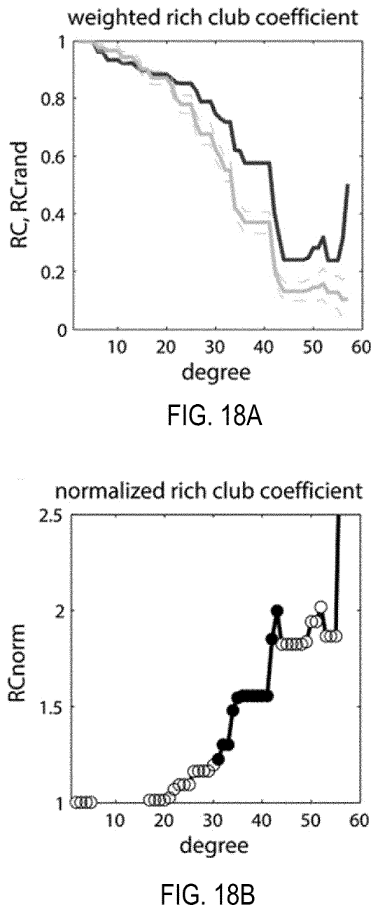

Single-Hemisphere Connection Number. The curation identified 731 rat cerebral nuclei association macroconnections (RCNAMs) as present and 1,051 as absent, between the 45 gray matter regions analyzed for the cerebral nuclei as a whole; this result yields a connection density of 41% (731/1,782). No adequate published data were found for 198 (10.0%) of all 1,980 (45²−45) possible association macroconnections; this result yields a matrix coverage (fill ratio) of 90% (FIG. 6). Assuming the curated literature representatively samples the 45-region matrix, the complete RCNAM dataset would contain ˜812 macroconnections (1,980×0.41), with a projected average of 18 input/output association macroconnections per cerebral nuclei region (812/45). For network analysis, values of “unclear” and “no data” are assigned to and combined with values in the “absent” category, resulting in a connection density of 37% (731/1,980). Considering only connections that have been unambiguously identified yields an average of input/output macroconnections of 16.2 (731/45), with significant variations for particular cerebral nuclei regions (input range 1-38, output range 0-36; FIGS. 7A and 7B). Individual regions show large variations in the ratio of the number of distinct inputs and outputs (their in degree and their out degree; FIG. 7A) as well as the aggregated strength of these inputs and outputs (their in strength and their out strength; FIG. 7B). Strong imbalance implies that some regions specialize as “receivers” of inputs and others as “senders” of outputs. The distribution of weight categories for the 731 connections reported as present is shown in FIG. 16A. The weight scale used for weighted network analysis is shown in FIG. 16B.

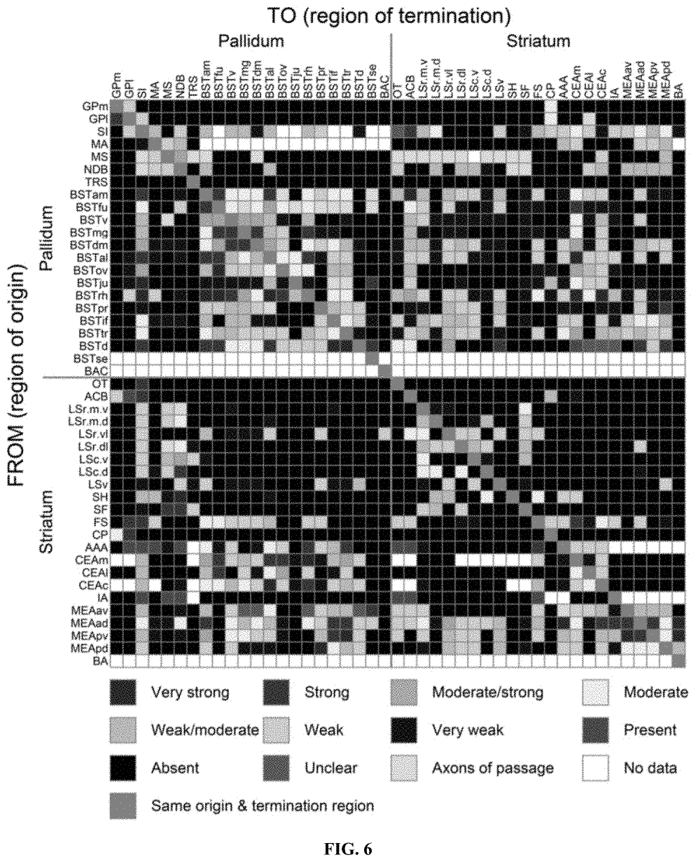

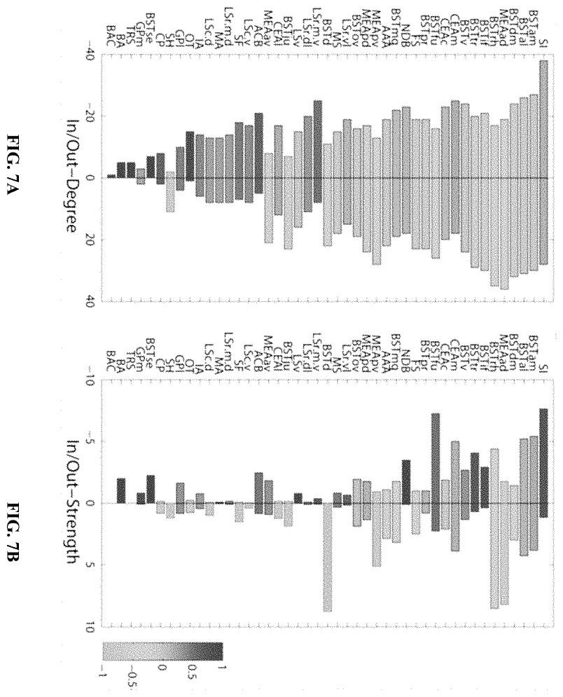

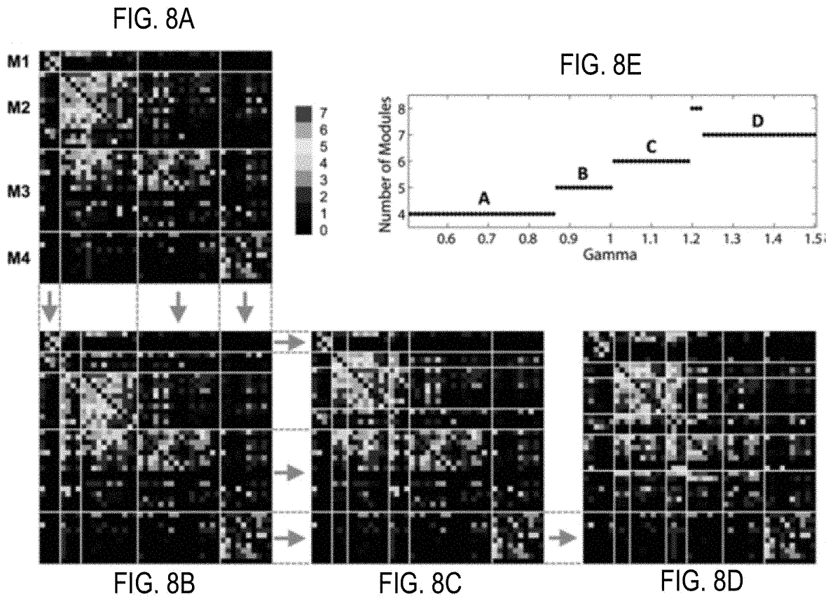

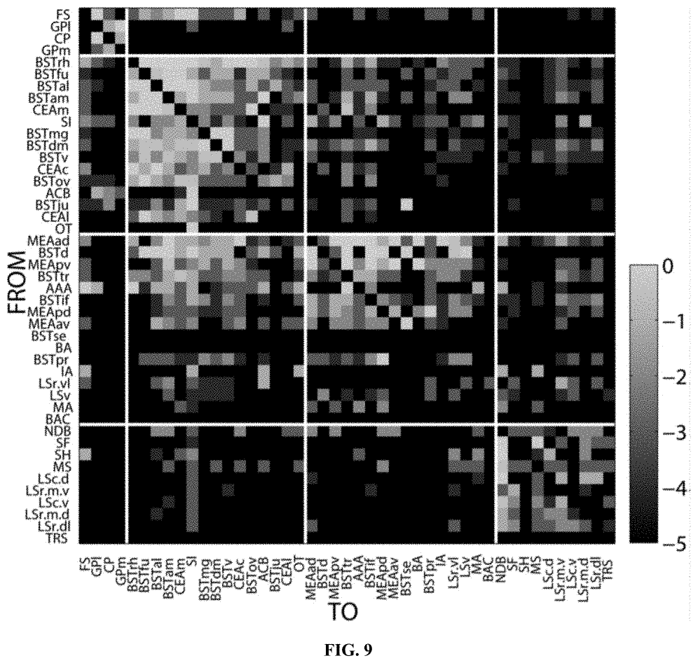

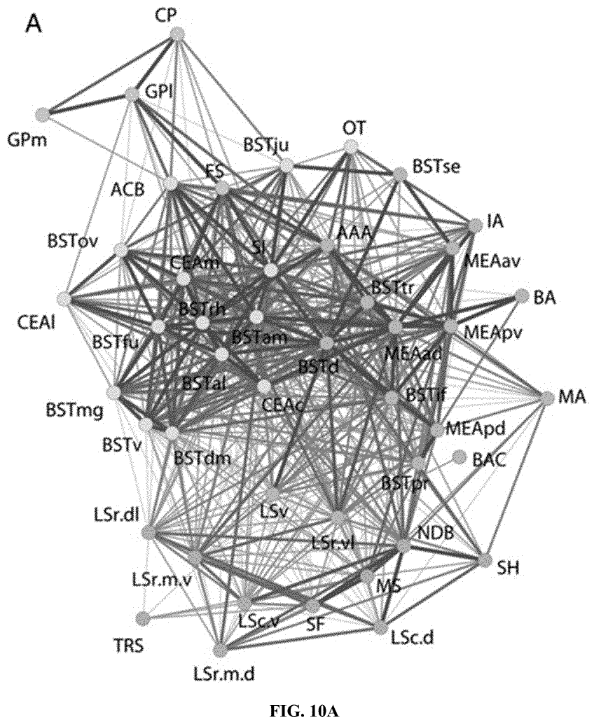

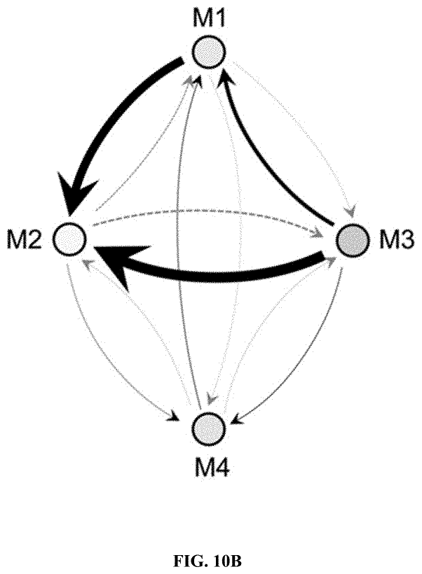

Network Analysis for Modules. For single-hemisphere interconnections, modules were detected by modularity maximization while systematically varying a spatial resolution parameter γ to assess module stability (Sporns, O. et al. (2016) Annu Rev Psychol. 67:613-640; Fortunato, S. et al. (2007) Proc Natl Acad Sci USA 104(1):36-41). Varying γ within the interval [0.5, 1.5] centered on the default setting of γ=1 resulted in four stable solutions (FIG. 8) with four to seven modules each. One module (M4) was stable across the entire range of γ, whereas modules M1-M3 were stable across most of the range. As the resolution parameter was increased toward finer and finer partitions, M3 split into two and then three submodules. The four-module solution was stable over the broadest range of γ and had the largest ratio of within-to-between module connection density in the range of γ examined here; hence, it was chosen for further analysis. The four-module solution with all regions and connections can be displayed as a weighted matrix (FIG. 9) or as a spring-embedded layout (FIG. 10A), and the regional composition of each module is provided below.

Module 1: (N=4, dorsal module or column) Blue Striatal fundus (FS), Lateral globus pallidus (GP1), Caudoputamen (CP), Medial globus pallidus GPm. Module 2: (N=15, ventral module or column), **Rhomboid nucleus of bed nuclei of terminal stria (BSTrh), Fusiform nucleus of bed nuclei of terminal stria (BSTfu), **Anterolateral area of bed nuclei of terminal stria (BSTal), **Anteromedial area of bed nuclei of terminal stria (BSTam), Medial part of central amygdalar nucleus (CEAm), **Innominate substance (SI), Magnocellular nucleus of bed nuclei of terminal stria (BSTmg), *Dorsomedial nucleus of bed nuclei of terminal stria (BSTdm), *Ventral nucleus of bed nuclei of terminal stria (BSTv), Capsular part of central amygdalar nucleus (CEAc), Oval nucleus of bed nuclei of terminal stria (BSTov), Accumbens nucleus (ACB), Juxtacapsular nucleus of bed nuclei of terminal stria (BSTju), Lateral part of central amygdalar nucleus (CEA1), Olfactory tubercle (OT); Module 3: (N=16, rostrocaudalmodule or column) **Anterodorsal part of medial amygdalar nucleus (MEAad), Dorsal nucleus of bed nuclei of terminal stria (BSTd), Posteroventralpart of medial amygdalar nucleus (MEApv), *Transverse nucleus of bed nuclei of terminal stria (BSTtr), Anterior amygdalar area (AAA), *Interfascicular nucleus of bed nuclei of terminal stria (B STif), Posterodorsalpart of medial amygdalar nucleus (MEApd), Anteroventralpart of medial amygdalar nucleus (MEAav), Stria extension of bed nuclei of stria terminalis (BSTse), Bed nucleus of accessory olfactory tract (BA), Principal nucleus of bed nuclei of terminal stria (BSTpr), Intercalated amygdalar nuclei (IA), Ventrolateral zone of rostral part of lateral septal nucleus (LSr.vl), Ventral part of lateral septalnucleus (LSv), Magnocellular nucleus (MA), Bed nucleus of anterior commissure (BAC); Module 4: (N=10, medial module or column) Diagonal band nucleus (NDB), Septofimbrial nucleus (SF), Septohippocampal nucleus (SH), Medial septal nucleus (MS), Dorsal zone of caudal part of lateral septal nucleus (LSc.d), Ventral region of medial zone of rostral part of lateral septal nucleus (LSr.m.v), Ventral zone of caudal part of lateral septalnucleus (LSc.v), Dorsal region of medial zone of rostral part of lateral septal nucleus (LSr.m.d), Dorsolateral zone of rostral part of lateral septalnucleus (LSr.dl), Triangular septalnucleus (TRS). Hub & rich club members are identified with a double asterisk. Members that are solely rich club members are identified with a single asterisk.

Connection Patterns. The simplest way to view the between-module interaction pattern is with aggregated between-module connections (FIG. 10B), which show that M1 and M3 are predominantly sending modules, M2 is predominantly a receiving module, and M4 is only weakly connected with the other modules. There are at least weak bidirectional connections between all four modules. Corresponding average module-by-module connection densities (binary and weighted) further illustrate this point and are provided in Table 1. Table 2 lists counts and percentages of connection classes by matrix block. Strongly asymmetric average connection weights between modules result in strongly directed (asymmetric) connection patterns among modules.

The weighted RCNAM network can be decomposed into a symmetric (fully reciprocated) and a directed (fully nonreciprocated) component (Squartini, T. et al. (2013) Sci Rep. 3:2729). To quantify the extent to which the distribution of connection weights in the RCNAM network is asymmetric, we computed the network's reciprocity p, a quantity that was then scaled with respect to a degree-preserving randomized null model. The reciprocity of the RCNAM network is ρ=0.262, which indicates a tendency to reciprocate that is less strong than that of the network of rat cortical association macroconnections (RCAMs) that yields ρ=0.331.

The 45 regions of the RCNAMnetwork comprise striatal (23 regions) and pallidal (22 regions) parts (FIG. 6). Whereas connections from pallidal to striatal regions are denser (45%) than striatal to pallidal connections (33%), the aggregated weight of striatal to pallidal connections is twice as strong as for the reverse direction. Stronger striatal to pallidal connections are also encountered within each of the four modules.

Efficiency, Hubs, and Rich Club. The overall network topology does not display classic small-world attributes, which is due to its relatively long path length (resulting in relatively low efficiency). Whereas clustering is greater than in randomized controls, the network is also significantly less efficient [clustering coefficient (CC)=0.0189 (0.0108±7.35×10⁻⁴; mean and SD of a population of random networks that were globally rewired while preserving the degree sequence; global efficiency (GE)=0.0837 (0.1351±0.0104)]. This trend prevails when nodes that lack identified association outputs [triangular septal nucleus (TRS), strial extension of bed nuclei of terminal stria (BSTse), bed nucleus of anterior commissure (BAC), and bed nucleus of accessory olfactory tract (BA)] are removed from the matrix [CC=0.0179 (0.0109±5.48×10⁻⁴), GE=0.0959 (0.1512±0.0116)]. The low efficiency of the RCNAM network is due to the existence of long paths between many of the networks' regions and modules. Whereas the mean path length between region pairs is 3.16 steps (computed from the weighted RCNAM network), 75 region pairs are linked by a minimal path with a length of 6 steps or greater. Among module pairs, modules M1 and M4 are topologically most distant from each other (see FIG. 10A) and are separated by, on average, 4.9 (M4→M1) and 4.6 (M1→M4) steps.

Centrality measures (degree, strength, betweenness, closeness) are summarized in FIG. 17. Regions innominate substance (SI), anteromedial area of bed nuclei of terminal stria (BSTam), anterolateral area of bed nuclei of terminal stria (BSTal), rhomboid nucleus of bed nuclei of terminal stria (BSTrh) and anterodorsal part of medial amygdalar nucleus (MEAad) (abbreviations in FIG. 6) rank in the top 20th percentile on all four measures, thus forming form a fully connected subgraph with an average connection weight of 0.39 (compared with a density of 37% and an average connection weight of 0.04 for the entire network). Four of the five candidate hubs are located in M2, with a sole hub (MEAad) in M3.

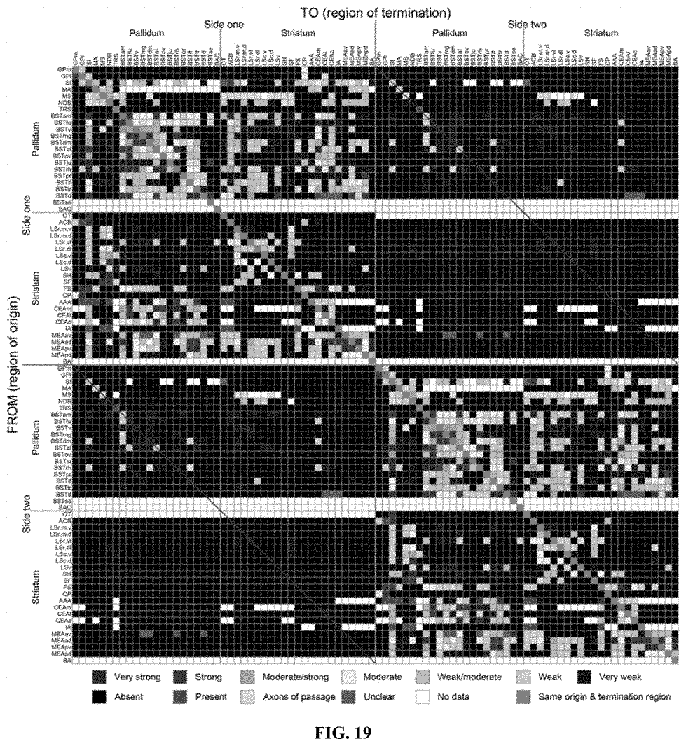

Rich club organization is present, as indicated by significantly greater density of connections among high-degree nodes compared with that in a degree-sequence—preserving null model (FIG. 19). Significance was assessed after correcting P values for multiple comparisons. The rich club shell where the corrected P value was minimal (P=3×10⁻⁷) contains nine member regions, including the five putative hubs listed above: SI, BSTam, ventral nucleus of bed nuclei of terminal stria (BSTv), dorsomedial nucleus of bed nuclei of terminal stria (BSTdm), BSTal, BSTrh, interfascicular nucleus of bed nuclei of terminal stria (BSTif), transverse nucleus of bed nuclei of terminal stria (BSTtr), and MEAad. Rich club members overlap exclusively with modules M2 and M3.

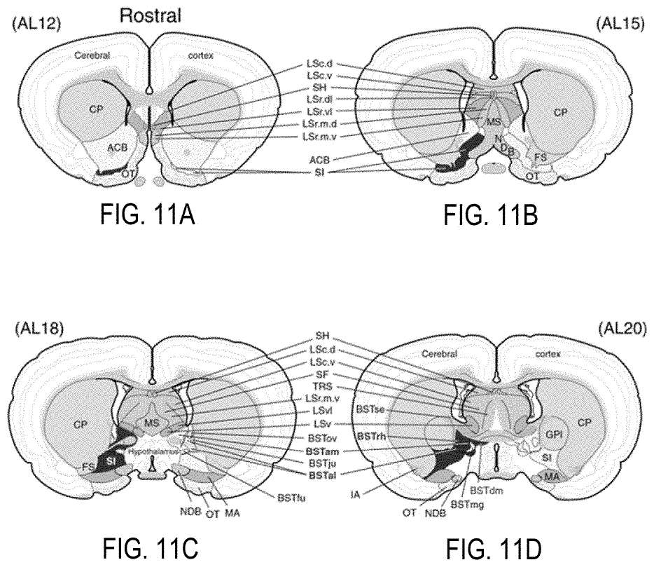

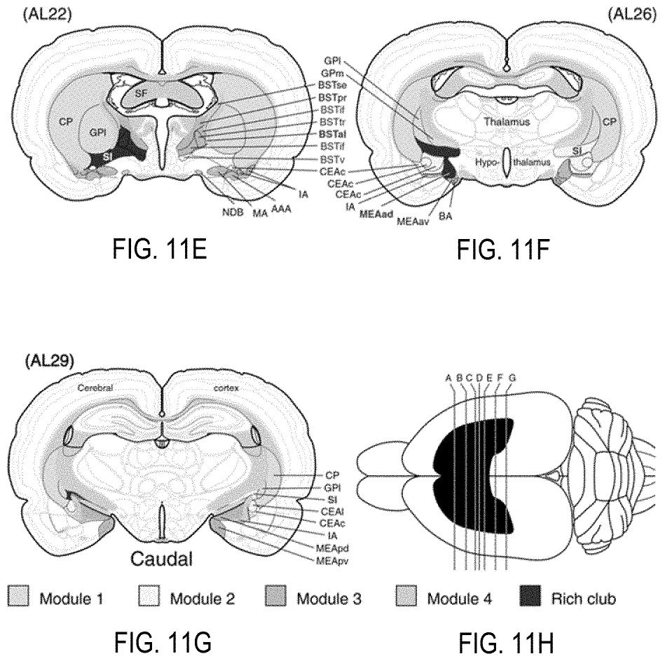

Topographic Arrangement of Modules, Rich Club, and Hubs. The spatial distribution of the four modules and their 45 individual regional components was mapped onto a standard brain atlas (Swanson, L. W. (2004) Brain Maps: Structure of the Rat Brain. A Laboratory Guide with Printed and Electronic Templates for Data, Models and Schematics (Elsevier, Amsterdam), 3rd Ed.) to distinguish whether module components are topographically either interdigitated or segregated (FIG. 11). Clearly, the modules form four spatially segregated longitudinal columns that may be described as dorsal (M1), ventral (M2), medial (M4), and rostrocaudal (M3), which consist of two segments (rostral and caudal) separated by a gap formed by region SI. The rich club also forms a spatially segregated mass of regions in M2 and M3 (FIG. 11), with the putative hubs nested within it.

The dorsal module (M1) corresponds to the classic dorsal striatopallidal system (Nieuwenhuys, R. et al. (2008) The Human Central Nervous System (Springer, Berlin), 4th Ed.; DeLong, M. R. et al. (2015) JAMA Neurol. 72(11):1354-1360), whereas the ventral module (M2) includes the classic ventral striatopallidum (Heimer, L. et al. (1975) Golgi Centennial Symposium Proceedings, ed Santini M (Raven, New York):177-193) and the central amygdalar nucleus and anterior division of bed nuclei of terminal stria (Dong H-W. et al. (2001) Brain Res Rev. 38(1-2):192-246). The caudal segment of M3 is formed by the medial amygdalar nucleus (striatal) and related regions, whereas the rostral segment is formed by the posterior division of bed nuclei of terminal stria (pallidal) and adjacent ventral parts of the lateral septal nucleus (Dong H-W. et al. (2001) Brain Res Rev. 38(1-2):192-246). Finally, the medial segment (M4) consists of the bulk of the septal region, which has both striatal and pallidal components (Swanson, L. W. (2000) Brain Res. 886(1-2):113-164; Risold, P. Y. et al. (1997) Brain Res Rev. 24(2-3):115-195).

Connections Between Hemispheres. Curation identified 96 commissural connections as present and 1,693 as absent (that is, of 1,789 connections for which adequate data were available, 5.4% are present, indicating a relatively sparse commissural network) between the 45 gray matter regions analyzed for all cerebral nuclei in each hemisphere. No adequate published data were found for 236 (11.7%) of all 2,025 (45²) possible commissural macroconnections, for a matrix coverage of 88.3%. Assuming the curated literature representatively samples the 45-region matrix, the complete rat cerebral nuclei commissural macroconnectome (RCNCM) dataset would contain ˜109 macroconnections (2,025×0.054), with an average of 2.4 output commissural macroconnections per cerebral nuclei region (109/45). The matrix of identified projections has a density of 96/2,025 (4.7%). Whereas this density corresponds to an average of 2.1 commissural macroconnections per region, their actual number varies over a broad range (input range 0-8; output range 0-13). All 96 identified commissural connections are in the very weak, weak, and weak to moderate weight categories. Twelve of 45 regions generated a homotopic commissural connection (to the same region on the contralateral side) and 84 of the commissural connections were heterotopic (to a different region on the contralateral side); all commissural connections were deemed symmetric with respect to the two sides because no evidence of right-left asymmetries was found.

A complete (both hemispheres) connection matrix for the cerebral nuclei is formed by the addition of commissural connections to the association connections (FIG. 19). Modules for the two-hemisphere (90-region) network were detected as for the single hemisphere, by varying the resolution parameter. By far the most stable solution contained eight modules, four in each hemisphere, matching the four-module solution found in the single-hemisphere analysis (FIG. 12).

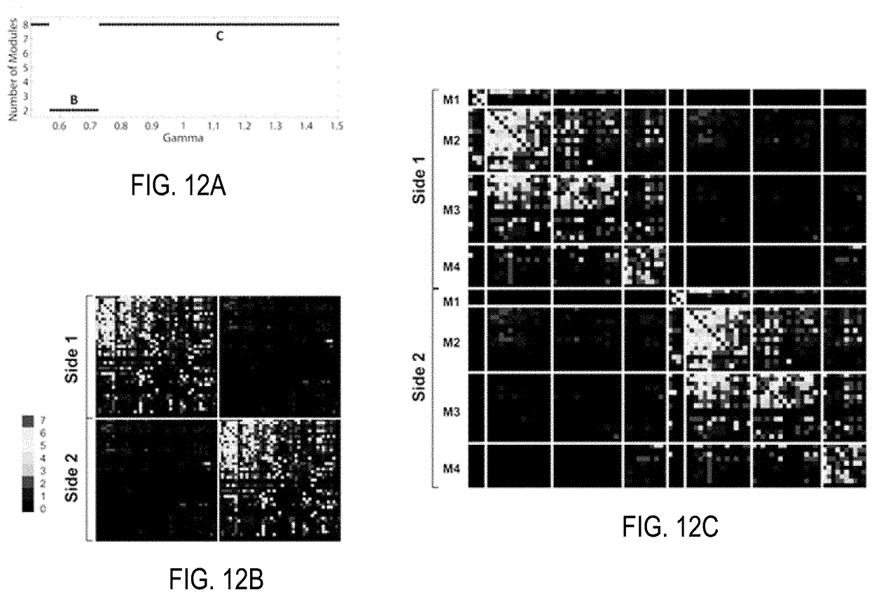

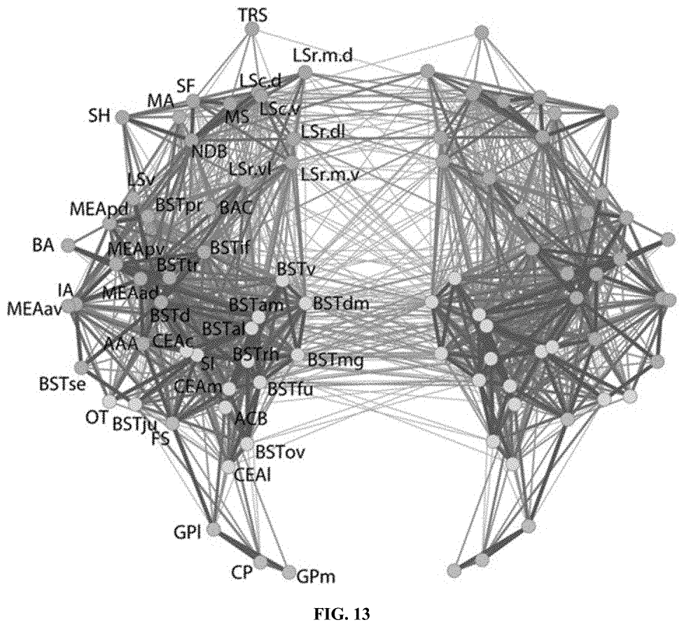

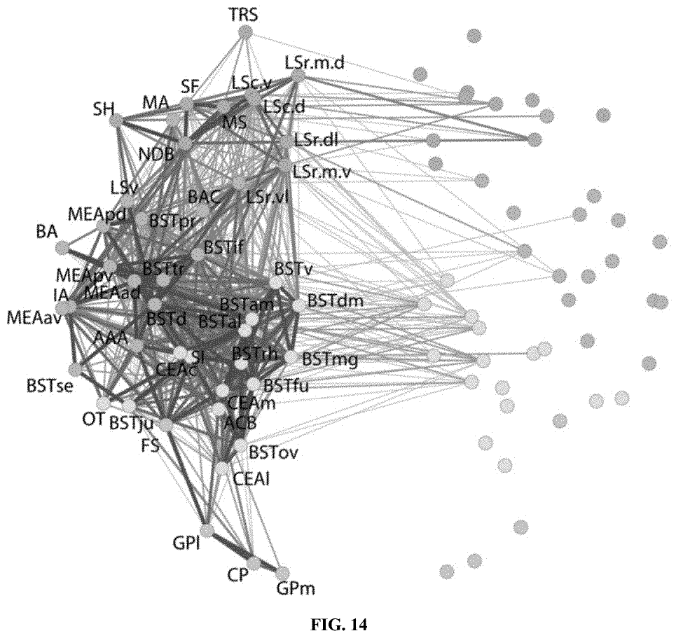

The density and weight of commissural connections by modules are summarized in Table 3 and a layout of the network for the two hemispheres is shown in FIG. 13. Module M1 maintains no commissural connections, whereas in contrast, M2 generates by far the most commissural connections, particularly with M2 and M3. Within modules M2-M4, regions BSTam, BSTdm, BSTal, BSTrh, and fusiform nucleus of bed nuclei of terminal stria (BSTfu) maintain the most commissural connections. The extent and distribution of association and commissural connections arising in one hemisphere is clearly illustrated by removing the association and commissural connections arising in the other hemisphere from the fully bilateral network (FIG. 14; compare with FIG. 8).

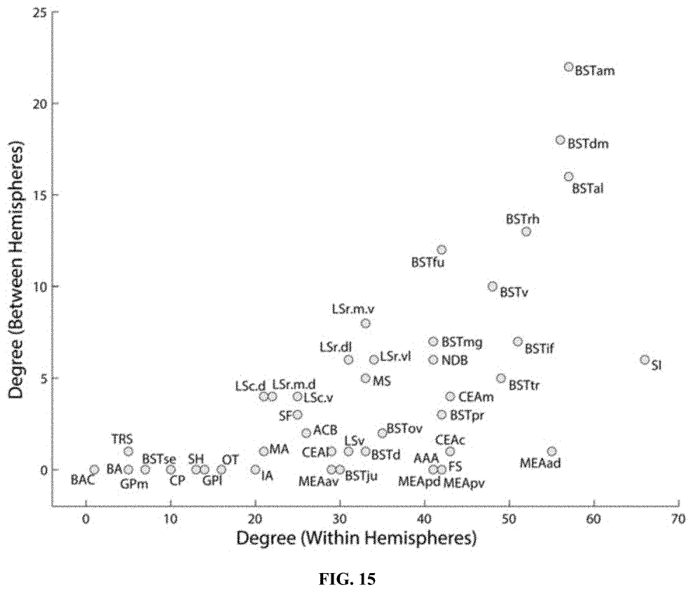

The number of intrahemispheric connections maintained by each region strongly correlates with the number of its commissural connections (FIG. 15). Computing the shortest paths for the 90-region network reveals that paths connecting modules across the two hemispheres vary greatly in length. M4(side1↔side2) is linked by relatively short paths, whereas paths between M1(side1↔side2) are among the longest in the entire network.

Inclusion of commissural connections (and inclusion of communication paths that span the two hemispheres) results in changed hub score rankings. Regions BSTal, BSTrh, and MEAad maintain their status as putative hubs with 80^{th} percentile rankings in all four centrality measures (degree, strength, betweenness, closeness), whereas regions SI and BSTam drop out.

### Discussion

This study yielded four basic results. First, the intracerebral nuclei network is arranged in four asymmetrically interconnected and topographically distinct network modules based on number and weight of connections. Second, the association connection subnetwork is much denser than the commissural subnetwork. Third, a topographically continuous band of rich club and hub regions (nodes) stretches through two of the modules. And fourth, the network as a whole does not show small-world organization.

A striking finding is that the cortical association network shows much stronger expression of small-world attributes (specifically short path length conferring high efficiency), which are not as clearly apparent in the cerebral nuclei association network. This finding was unexpected because small-world topology was a common feature in earlier analyses of nervous system organization, many of them carried out on representations of cortical networks.

Another major finding with important implications for network modeling is that the sign of most cerebral cortical association connections is presumably positive (excitatory) whereas the sign of most cerebral nuclei association connections is presumably negative (inhibitory) (Nieuwenhuys, R. et al. (2008) The Human Central Nervous System (Springer, Berlin), 4th Ed.). These differences in architectural features may reflect differences in function, with the small-world topology of cortico-cortical networks promoting the integration of information (requiring globally short paths) whereas intracerebral nuclei networks mediate the flow of neural signals between cortex and subcortical regions involved in controlling specific behaviors (biased toward parallel and independent paths).

The structure—function significance of the four network modules in each hemisphere and the connections within and between modules (in both hemispheres; FIG. 12) are not immediately obvious. However, one generalization seems clear: Module M1 corresponds to the classical striatopallidum, which receives its major input from isocortex and is involved in somatomotor control mechanisms (Nieuwenhuys, R. et al. (2008) The Human Central Nervous System (Springer, Berlin), 4th Ed.; DeLong, M. R. et al. (2015) JAMA Neurol. 72(11):1354-1360), whereas the other three modules (M2-M4) receive their major input from limbic cortex and are involved in motivated behavioral mechanisms (DeLong, M. R. et al. (2015) JAMA Neurol. 72(11):1354-1360; Heimer, L. et al. (1975) Golgi Centennial Symposium Proceedings, ed Santini M (Raven, New York):177-193; Swanson, L. W. (2000) Brain Res. 886(1-2):113-164; Dong H-W. et al. (2001) Brain Res Rev. 38(1-2):192-246; Risold, P. Y. et al. (1997) Brain Res Rev. 24(2-3):115-195). As broad generalizations, module 2 contains the central amygdalar nucleus and anterior division of the bed nuclei of the terminal stria and has been most notably implicated in homeostatic behaviors (for example, eating and drinking) and anxiety (Dong H-W. et al. (2001) Brain Res Rev. 38(1-2):192-246; Dong, H. W. et al. (2003) J Comp Neurol. 463(4):434-472; Dong, H-W. et al. (2004) J Comp Neurol. 468(2):277-298; Dong, H-W. et al. (2006) J Comp Neurol. 494(1):75-107; Dong, H-W. et al. (2006) J Comp Neurol. 494(1):142-178); module 3 contains the medial amygdalar nucleus and posterior division of the bed nuclei of the terminal stria and has been implicated most notably in social interactions and responding to threats in the environment (Dong H-W. et al. (2001) Brain Res Rev. 38(1-2):192-246; Canteras, N. S. et al. (1995) J Comp Neurol. 360(2):213-245; Dong, H-W. et al. (2004) J Comp Neurol 471(4):396-433); and module 4 receives its major inputs from the hippocampus and may thus play a role in spatial and mnemonic influences on motivated behavior in general (Risold, P. Y. et al. (1997) Brain Res Rev. 24(2-3):115-195).

Furthermore, the nine members of the rich club (and the subset of putative hubs within it: SI, BSTam, BSTv, BSTdm, BSTal, BSTrh, BSTif, BSTtr, and MEAad) all reside in M2 and M3, and the extrinsic connections of the rich club predict that collectively they coordinate somatic, autonomic, and neuroendocrine responses in motivated survival behaviors, including reproductive, fight or flight, eating and drinking, and foraging (Heimer, L. et al. (1975) Golgi Centennial Symposium Proceedings, ed Santini M (Raven, New York):177-193; Dong, H. W. et al. (2003) J Comp Neurol. 463(4):434-472; Dong, H-W. et al. (2004) J Comp Neurol. 468(2):277-298; Dong, H-W. et al. (2006) J Comp Neurol. 494(1):75-107; Dong, H-W. et al. (2006) J Comp Neurol. 494(1):142-178; Canteras, N. S. et al. (1995) J Comp Neurol. 360(2):213-245; Dong, H-W. et al. (2004) J Comp Neurol 471(4):396-433).

### Materials and Methods

All relevant data in the primary literature were interpreted in the only available standard, hierarchically organized, annotated nomenclature for the rat brain (Table 4), using descriptive nomenclature defined in the foundational model of connectivity (Swanson, L. W. et al. (2010) Proc Natl Acad Sci USA 107(48):20610-20617; Brown, R. A. et al. (2013) J Comp Neurol 521(13):2889-2906). Association and commissural connection reports were assigned ranked qualitative connection weights based on pathway tracing methodology, injection site location and extent, and anatomical density.

Cerebral Nuclei Histological Parcellation Granularity. To facilitate comparative analysis of rat connection data, they were collated with reference to a standard rat brain atlas (Swanson, L. W. (2004) Brain Maps: Structure of the Rat Brain. A Laboratory Guide with Printed and Electronic Templates for Data, Models and Schematics (Elsevier, Amsterdam), 3rd Ed.), but with gray matter regions of the rat central nervous system (CNS) arranged with respect to a CNS hierarchical nomenclature in a strictly topographic order as outlined elsewhere (Swanson, L. W. (2015) Neuroanatomical Terminology: A Lexicon of Classical Origins and Historical Foundations (Oxford Univ Press, Oxford)), rather than in a structure—function order followed earlier (Bota, M. et al. (2015) Proc Natl Acad Sci USA 112(16):E2093-E2101; Swanson, L. W. (2004) Brain Maps: Structure of the Rat Brain. A Laboratory Guide with Printed and Electronic Templates for Data, Models and Schematics (Elsevier, Amsterdam), 3rd Ed.; Swanson, L. W. (2003) Brain Architecture: Understanding the Basic Plan (Oxford Univ Press, Oxford)). In addition, the CNS hierarchical levels of gray matter regions and subregions were explicitly recognized as comparable (respectively) to the species and subspecies levels in animal taxonomy (International Commission on Zoological Nomenclature (1999) International Code of Zoological Nomenclature (International Trust for Zoological Nomenclature, London)) (Table 4). This nomenclature scheme recognizes 45 gray matter regions in the rat cerebral nuclei, all of which are included in the present analysis.

Connection Report Collation and Selection for Network Analysis. Our methodology for expertly collating connectional data from the primary neuroanatomical research literature is summarized here. First, the primary literature was searched to find the best available connection data, from which connection reports were created. Several criteria were used to assess the quality of connection data: These included the validity of the experimental pathway tracing method used, restriction of the pathway tracer injection site to the gray matter region of interest, injection site coverage of the region of interest, and thoroughness of description of the connection. Next, for each possible connection in the connectome for which data were available, a single connection report was selected as best representative of the connection (using the criteria noted above). If more than one connection report was created for a given connection (depending on the availability of data in the primary literature), then, all else being equal, the connection report with the highest connection weight was selected. Finally, the weight of each selected connection report was used to populate a connection matrix that was used for subsequent network analysis.

The process of collation was considerably aided by the use of a dedicated data entry platform (Axiome) designed as a spreadsheet template for use with Microsoft Excel. The template facilitates speed and accuracy of data entry by using data validation and conditional formatting rules and a highly structured and guided user-friendly interface.

The sequence of tabulated connection reports follows the list of regions in Table 4. When multiple connection reports for a connection of interest were found, one was chosen for network analysis, with a selected value of “yes”. Abbreviations for pathway tracers: ARGM, autoradiographic method; BDA-10K, biotinylated dextran amine, Mr 10,000); BDA-3K, biotinylated dextran amine, Mr 3,000); CTB, cholera toxin B subunit; Fluoro-Gold; HRP, horseradish peroxidase; neurobiotin; PHAL, Phaseolus vulgaris leucoagglutinin; True Blue; WGA-HRP, horseradish peroxidase conjugated to wheat germ agglutinin.

Connection Weight Scaling Methodology for Network Analysis. There are almost no quantitative data available in the literature for the rat macroconnections used in this analysis. Therefore, ranked qualitative connection weights from the literature were divided into 12 value categories. In ascending order, they are no data, unclear, absent, axons of passage, very weak, weak, weak to moderate, present (value unreported), moderate, moderate to strong, strong, and very strong. For the purposes of our network analysis, reports of axons of passage were assigned a weight of “weak,” and connections for which the reported value was entered as “present” (weight unreported) were assigned a weight of “moderate.” When network analysis was applied to the dataset, the category values of unclear and no data were assigned to the absent category. Thus, the set of ranked qualitative values used for network analysis included 8 values (7 weights and 0 for absent) that were considered for our purposes to form an ordinal scale. The ranked qualitative connection weights were then transformed to approximately logarithmically spaced weights for network analysis, using a 104 exponential scale.

Network Analysis Methods. Network analyses were carried out on the directed and log-weighted rat intracerebral nuclei macroconnection (RiCNM) matrix (FIGS. 1, 4, and 14), using tools collected in the Brain Connectivity Toolbox (www.brainconnectivity-toolbox.net). Detailed descriptions of most network measures can be found in Rubinov, M. et al. (2010) Neuroimage 52(3):1059-1069. Rat cerebral nuclei gray matter regions are referred to as nodes of the RiCNM network.

For detection of optimal module partitions we implemented the Louvain algorithm (Blondel, V. et al. (2008) J Stat Mech.: P10008) for modularity maximization (Sporns, O. et al. (2016) Annu Rev Psychol. 67:613-640; Newman, M. E. J. et al. (2004) Phys Rev E Stat Nonlin Soft Matter Phys 69(2 Pt 2):026113), including a resolution parameter γ designed to address a known limitation of modularity optimization, the resolution limit (Fortunato, S. et al. (2007) Proc Natl Acad Sci USA 104(1):36-41). Varying γ effectively allows the detection of modules that range over several spatial scales. In our study, the parameter γ was varied over a range of γ=[0.5-1.5], an interval centered on the default setting of γ=1; higher and lower settings of γ yielded mostly unstable solutions with unrealistically low or high numbers of modules. Robust module partitions are expected to remain stable over a broad range of settings of γ; that is, they should be insensitive to small variations in spatial scale. Modularity was optimized 1,000 times for each setting of γ and encountered very little degeneracy in the distribution of solutions across these 1,000 iterations. Hence the globally optimal module partition at each level of γ were selected for further analysis.

Analyses of global network metrics such as clustering and efficiency, reciprocity, and rich club organization were statistically evaluated by comparison with a degree-sequence—preserving distribution of null models, as in previous work (Bota, M. et al. (2015) Proc Natl Acad Sci USA 112(16):E2093-E2101). Rewiring of the networks composing the random null model followed a commonly used procedure equivalent to a Markov switching algorithm (Maslov, S. et al. (2002) Science 296(5569):910-913) that preserves the number of incoming and outgoing connections on all nodes.

Network hubs were determined on the basis of aggregated rankings across several distinct nodal centrality measures. These measures were node degree, node strength, node betweenness centrality, and closeness centrality. The node degree is defined as the sum of all incoming and outgoing connections per node. The node strength is defined as the total weight of all incoming and outgoing connections per node (computed from the weighted connection matrix). The node betweenness expresses the fraction of shortest paths that pass through each node. Closeness was calculated as the average of the row and column sum of the network's distance matrix. Betweenness and closeness were both derived from the weighted connection matrix, after converting connection weights to lengths, using an inverse transform. After ranking nodes on each of the four metrics, an aggregate “hub score” was determined for each node, expressing the number of metrics for which each node appeared in the top 20% (top nine nodes).

Rich club organization refers to a simple property that is shared by many, but not all, complex biological networks (Colizza, V. et al. (2006) Nat Phys 2:110-115)—the propensity of highly connected nodes (that is, nodes with high degree) to also be densely connected to each other, more so than expected by chance. This analysis proceeded along the following steps, in line with previous work (Bota, M. et al. (2015) Proc Natl Acad Sci USA 112(16):E2093-E2101). First, for each value of node degree k, the total sum of the weights W_{>k} between all nodes with degree k or higher was determined. No distinction is made between incoming and outgoing connection weights. Next, the weighted rich club coefficient Φ^{w}(k) was computed as the ratio between W_{>k} and the sum of the weights of the strongest E_{>k} connections across the whole network. The weighted rich club coefficient was then normalized against a set of 10,000 randomly rewired networks, preserving network size, density, and degree sequence (see above). Comparison of the rich club coefficient of the empirical network to this random null distribution was then subjected to significance testing. To correct for multiple comparisons over the range of degrees k examined, false-discovery rate correction was performed (Benjamini, Y. et al. (1995) J R Stat Soc B 57:289-300), at a false discovery rate of 0.001.

Reciprocity was assessed following the approach by Squartini et al. (Squartini, T. et al. (2013) Sci Rep. 3:2729). Briefly, the network was decomposed into a symmetric (reciprocated) part and an asymmetric (nonreciprocated) part. The weighted reciprocity of the network was then computed as the ratio between the total reciprocated weight (the sum of all of the weights contained in the reciprocated part) and the total weight of the network. This quantity was then scaled relative to the average weighted reciprocity derived from the degree-sequence—preserving random null model (see above). The resulting metric ρ indicates the tendency of the network to reciprocate (ρ>0) or to avoid reciprocation (ρ<0). In the case of ρ>0, a higher value of ρ indicates a stronger tendency to reciprocate.

TABLE 1
Association connection density by module (binary and weighted)

| | To | | | |
|---|---|---|---|---|
| | M1 | M2 | M3 | M4 |
| From | | | | |
| Binary | | | | |
| M1 | 0.6667 | 0.2500 | 0.0938 | 0.0500 |
| M2 | 0.3833 | 0.7762 | 0.3708 | 0.3200 |
| M3 | 0.1250 | 0.4833 | 0.4500 | 0.3000 |
| M4 | 0.0250 | 0.1600 | 0.1125 | 0.6000 |
| Weighted | | | | |
| M1 | 0.2064 | 0.0291 | 0.0001 | 0.0000 |
| M2 | 0.0033 | 0.1808 | 0.0072 | 0.0009 |
| M3 | 0.0143 | 0.0334 | 0.0846 | 0.0032 |
| M4 | 0.0019 | 0.0004 | 0.0005 | 0.0510 |

TABLE 2
Association connection weight categories by module
(counts and percentages)

| | | | | | | |
|---|---|---|---|---|---|---|
| Vol wt | wt | wt/m | m | m/s | S | vs |

Counts

| | | | | | | | |
|---|---|---|---|---|---|---|---|
| M1 ↔ M1 | 0 | 2 | 0 | 3 | 0 | 3 | 0 |
| M1 → M2 | 1 | 8 | 1 | 3 | 0 | 2 | 0 |
| M1 → M3 | 1 | 5 | 0 | 0 | 0 | 0 | 0 |
| M1 → M4 | 2 | 0 | 0 | 0 | 0 | 0 | 0 |
| M2 → M1 | 9 | 8 | 4 | 2 | 0 | 0 | 0 |
| M2 ↔ M2 | 21 | 29 | 29 | 32 | 15 | 25 | 12 |
| M2 → M3 | 31 | 38 | 11 | 7 | 1 | 1 | 0 |
| M2 → M4 | 31 | 12 | 4 | 1 | 0 | 0 | 0 |
| M3 → M1 | 0 | 4 | 1 | 2 | 0 | 1 | 0 |
| M3 → M2 | 24 | 40 | 17 | 26 | 2 | 7 | 0 |
| M3 ↔ M3 | 14 | 31 | 27 | 12 | 2 | 14 | 8 |
| M3 → M4 | 17 | 21 | 4 | 6 | 0 | 0 | 0 |
| M4 → M1 | 0 | 0 | 0 | 1 | 0 | 0 | 0 |
| M4 → M2 | 5 | 15 | 4 | 0 | 0 | 0 | 0 |
| M4 → M3 | 4 | 7 | 7 | 0 | 0 | 0 | 0 |
| M4 ↔ M4 | 6 | 22 | 12 | 8 | 2 | 3 | 1 |

%

| | | | | | | | |
|---|---|---|---|---|---|---|---|
| M1 ↔ M1 | 0 | 25 | 0 | 38 | 0 | 38 | 0 |
| M1 → M2 | 7 | 53 | 7 | 20 | 0 | 13 | 0 |
| M1 → M3 | 17 | 83 | 0 | 0 | 0 | 0 | 0 |
| M1 → M4 | 100 | 0 | 0 | 0 | 0 | 0 | 0 |
| M2 → M1 | 39 | 35 | 17 | 9 | 0 | 0 | 0 |
| M2 ↔ M2 | 13 | 18 | 18 | 20 | 9 | 15 | 7 |
| M2 → M3 | 35 | 43 | 12 | 8 | 1 | 1 | 0 |
| M2 → M4 | 65 | 25 | 8 | 2 | 0 | 0 | 0 |
| M3 → M1 | 0 | 50 | 13 | 25 | 0 | 13 | 0 |
| M3 → M2 | 21 | 34 | 15 | 22 | 2 | 6 | 0 |
| M3 ↔ M3 | 13 | 29 | 25 | 11 | 2 | 13 | 7 |
| M3 → M4 | 35 | 44 | 8 | 13 | 0 | 0 | 0 |
| M4 → M1 | 0 | 0 | 0 | 100 | 0 | 0 | 0 |
| M4 → M2 | 21 | 63 | 17 | 0 | 0 | 0 | 0 |
| M4 → M3 | 22 | 39 | 39 | 0 | 0 | 0 | 0 |
| M4 ↔ M4 | 11 | 41 | 22 | 15 | 4 | 6 | 2 |

m, moderate; m/s, moderate/strong; s, strong; vs, very strong; vw, very weak; w, weak; w/m, weak/moderate.

TABLE 3
Commissural connection density by module (binary and weighted)

| | To side 1 | | | |
|---|---|---|---|---|
| | M1 | M2 | M3 | M4 |
| From side 2 | | | | |
| Binary | | | | |
| M1 | 0 | 0 | 0 | 0 |
| M2 | 0 | 0.1952 | 0.0458 | 0.0667 |
| M3 | 0 | 0.0167 | 0.0042 | 0.0313 |
| M4 | 0 | 0 | 0 | 0.1333 |
| Weighted × 10⁻³ | | | | |
| M1 | 0 | 0 | 0 | 0 |
| M2 | 0 | 0.0667 | 0.0121 | 0.0067 |
| M3 | 0 | 0.0017 | 0.0004 | 0.0087 |
| M4 | 0 | 0 | 0 | 0.2733 |

TABLE 4
Brain Maps 4 Hierarchical Nomenclature Table

Endbrain (Kuhlenbeck, 1927) (EB) or Cerebrum (Obersteiner & Hill,
1900) (CH)
Cerebral nuclei (Swanson, 2000a) (CNU)
Pallidum (Swanson, 2000a) (PAL)
Globus pallidus (Burdach, 1822) (GP)
*Medial globus pallidus (>1840) (GPm)
*Lateral globus pallidus (>1840) (GPl)
*Innominate substance (Schwalbe, 1881) or Substantia innominata
(Schwalbe, 1881) (SI)
*Magnocellular nucleus (Swanson, 2004) (MA)
Medial septal complex (Swanson et al., 1987) (MSC)
*Medial septal nucleus (>1840) (MS)
*Diagonal band nucleus (>1840) (NDB)
*Triangular septal nucleus (>1840) (TRS)
bed nucleus of stria medullaris (Risold & Swanson, 1995) (BSM)
Bed nuclei of terminal stria (Gurdjian, 1925)15 (BST)
Anterior division (Ju & Swanson, 1989) (BSTa)
*Anteromedial area (Dong & Swanson, 2006c) (BSTam) *Fusiform
nucleus (Ju & Swanson, 1989)18 (BSTfu)
*Ventral nucleus (Ju & Swanson, 1989) (BSTv)
*Magnocellular nucleus (Ju & Swanson, 1989) (BSTmg)
*Dorsomedial nucleus (Ju & Swanson, 1989) (BSTdm)
*Anterolateral area (Swanson, 2004) (BSTal)
*Oval nucleus (Ju & Swanson, 1989) (BSTov)
*Juxtacapsular nucleus (McDonald, 1983) (BSTju) *Rhomboid
nucleus (Ju & Swanson, 1989) (BSTrh)
Posterior division (Ju & Swanson, 1989) (BSTp)
*Principal nucleus (Ju & Swanson, 1989) (BSTpr)
cell-sparse zone (Ju & Swanson, 1989) (BSTsz)
*Interfascicular nucleus (Ju & Swanson, 1989) (BSTif)
premedullary nucleus (Ju & Swanson, 1989) (BSTpm)
*Transverse nucleus (Ju & Swanson, 1989) (BSTtr)
*Dorsal nucleus (Ju & Swanson, 1989) (BSTd)
*Strial extension (Ju & Swanson, 1989) (BSTse)
*Bed nucleus of anterior commissure (Gurdjian, 1925) (BAC)
Striatum (Swanson, 2000a) (STR)
*Olfactory tubercle (Calleja, 1893) (OT)
molecular layer (>1840) (OT1)
pyramidal layer (>1840) (0T2)
polymorph layer (>1840) (0T3)
islands of Calleja (>1840) (isl)
major island of Calleja (>1840) (islm)
*Accumbens nucleus (Ziehen, 1897-1901) (ACB)
Lateral septal complex (Risold & Swanson, 1997a) (LSX)
Lateral septal nucleus (Cajal, 1909-1911) (LS)
Rostral (rostroventral) part (Risold & Swanson, 1997a)
(LSr)
Medial zone (Risold & Swanson, 1997a) (LSr.m)
*Ventral region (Risold & Swanson, 1997a)
(LSr.m.v)
rostral domain (Risold & Swanson, 1997a)
(LSr.m.v.r)
caudal domain (Risold & Swanson, 1997a)
(LSr.m.v.c)
*Dorsal region (Risold & Swanson, 1997a)
(LSr.m.d)
*Ventrolateral zone (Risold & Swanson, 1997a) (LSr.vl)
ventral region (Risold & Swanson, 1997a)
(LSr.vl.v)
dorsal region (Risold & Swanson, 1997a)
(LSr.vl.d)
medial domain (Risold & Swanson, 1997a)
(LSr.vl.d.m)
lateral domain (Risold & Swanson, 1997a)
(LSr.vl.d.l)
*Dorsolateral zone (Risold & Swanson, 1997a) (LSr.dl)
medial region (Risold & Swanson, 1997a)
(LSr.dl.m)
ventral domain (Risold & Swanson, 1997a)
(LSr.dl.m.v)
dorsal domain (Risold & Swanson, 1997a)
(LSr.dl.m.d)
lateral region (Risold & Swanson, 1997a) (LSr.dl.l)
ventral domain (Risold & Swanson, 1997a)
(LSr.dl.l.v)
dorsal domain (Risold & Swanson, 1997a)
(LSr.dl.l.d)
Caudal (caudodorsal) part (Risold & Swanson, 1997a) (LSc)
*Ventral zone (Risold & Swanson, 1997a) (LSc.v)
medial region (Risold & Swanson, 1997a)
(LSc.v.m)
ventral domain (Risold & Swanson, 1997a)
(LSc.v.m.v)
dorsal domain (Risold & Swanson, 1997a)
(LSc.v.m.d)
intermediate region (Risold & Swanson, 1997a)
(LSc.v.i)
lateral region (Risold & Swanson, 1997a) (LSc.v.l)
ventral domain (Risold & Swanson, 1997a)
(LSc.v.l.v)
dorsal domain (Risold & Swanson, 1997a)
(LSc.v.l.d)
*Dorsal zone (Risold & Swanson, 1997a) (LSc.d)
rostral region (Risold & Swanson, 1997a) (LSc.d.r)
dorsal region (Risold & Swanson, 1997a) (LSc.d.d)
lateral region (Risold & Swanson, 1997a) (LSc.d.l)
ventral region (Risold & Swanson, 1997a)
(LSc.d.v)
*Ventral part (Risold & Swanson, 1997a) (LSv)
*Septohippocampal nucleus (>1840) (SH)
*Septofimbrial nucleus (>1840) (SF)
*Striatal fundus (>1840) (FS)
*Caudoputamen (Heimer & Wilson, 1975) (CP)
*Anterior amygdalar area (Gurdjian, 1928) (AAA)
Central amygdalar nucleus (Johnston, 1923) (CEA)
*Medial part (McDonald, 1982) (CEAm)
*Lateral part (Swanson, 1992) (CEAl)
*Capsular part (McDonald, 1982) (CEAc)
*Intercalated amygdalar nuclei (>1840) (IA)
Medial amygdalar nucleus (Johnston, 1923) (MEA)
*Anteroventral part (>1840) (MEAav)
*Anterodorsal part (>1840) (MEAad)
*Posteroventral part (>1840) (MEApv)
*Posterodorsal part (>1840) (MEApd)
sublayer a (>1840) (MEApd-a)
sublayer b (>1840) (MEApd-b)
sublayer c (>1840) (MEApd-c)
*Bed nucleus of accessory olfactory tract (Scialia & Winans,
1975) (BA)

Table 4 is a rearrangement of the cerebral nuclei parts in table B of Brain Maps 3 (Herrick, C. J. (1910) J Comp Neurol Psychol 20(5):413-547), with updated annotations as endnotes. The nomenclature hierarchy in Brain Maps 3 was arranged according to a structure—function model of the central nervous system (Nieuwenhuys, R. et al. (2008) The Human Central Nervous System (Springer, Berlin), 4th Ed.). The nomenclature in this beta version of Brain Maps 4 is arranged according to a strictly topographic model of the central nervous system, as adapted for the rat. Region level terms are marked with an asterisk and subregion-level terms under them are italicized.

TABLE 5A
Data matrices for the association and commissural connections of the rat cerebral
nuclei derived from collation of connection reports from the primary literature

| | GPm | GPl | SI | MA | MS | NDB | TRS | BSTam | BSTfu | BSTv | BSTmg |
|---|---|---|---|---|---|---|---|---|---|---|---|
| GPm | 0 | 2 | 0 | 0 | 0 | 0 | 0 | 0 | 0 | 0 | 0 |
| GPl | 6 | 0 | 2 | 0 | 0 | 0 | 0 | 0 | 0 | 0 | 0 |
| SI | 0 | 2 | 0 | 3 | 0 | 2 | 0 | 3 | 0 | 3 | 3 |
| MA | 0 | 0 | 1 | 0 | 2 | 3 | 0 | 0 | 0 | 0 | 0 |
| MS | 0 | 0 | 2 | 2 | 0 | 5 | 2 | 2 | 0 | 0 | 0 |
| NDB | 0 | 0 | 2 | 2 | 2 | 0 | 0 | 3 | 0 | 0 | 0 |
| TRS | 0 | 0 | 0 | 0 | 0 | 0 | 0 | 0 | 0 | 0 | 0 |
| BSTam | 0 | 0 | 6 | 0 | 0 | 1 | 0 | 0 | 6 | 4 | 4 |
| BSTfu | 0 | 0 | 4 | 0 | 0 | 2 | 0 | 6 | 0 | 4 | 2 |
| BSTv | 0 | 0 | 3 | 0 | 2 | 0 | 0 | 5 | 5 | 0 | 5 |
| BSTmg | 0 | 0 | 3 | 0 | 0 | 0 | 0 | 4 | 6 | 6 | 0 |
| BSTdm | 0 | 0 | 5 | 1 | 1 | 1 | 0 | 4 | 5 | 6 | 6 |
| BSTal | 0 | 1 | 6 | 1 | 0 | 1 | 0 | 6 | 6 | 3 | 4 |
| BSTov | 0 | 1 | 5 | 0 | 0 | 0 | 0 | 3 | 7 | 2 | 2 |
| BSTju | 0 | 1 | 7 | 0 | 0 | 0 | 0 | 1 | 1 | 1 | 1 |
| BSTrh | 0 | 2 | 7 | 2 | 0 | 1 | 0 | 7 | 7 | 6 | 5 |
| BSTpr | 0 | 0 | 1 | 0 | 0 | 0 | 0 | 2 | 2 | 3 | 3 |
| BSTif | 0 | 0 | 4 | 1 | 1 | 1 | 0 | 3 | 0 | 3 | 3 |
| BSTtr | 0 | 0 | 4 | 0 | 0 | 0 | 0 | 4 | 3 | 3 | 2 |
| BSTd | 0 | 0 | 6 | 0 | 0 | 0 | 0 | 6 | 6 | 4 | 4 |
| BSTse | 0 | 0 | 0 | 0 | 0 | 0 | 0 | 0 | 0 | 0 | 0 |
| BAC | 0 | 0 | 0 | 0 | 0 | 0 | 0 | 0 | 0 | 0 | 0 |
| OT | 0 | 0 | 6 | 0 | 0 | 0 | 0 | 0 | 0 | 0 | 0 |
| ACB | 2 | 4 | 6 | 0 | 0 | 0 | 0 | 0 | 0 | 0 | 0 |
| LSr.m.v | 0 | 0 | 2 | 0 | 2 | 2 | 0 | 0 | 0 | 1 | 0 |
| LSr.m.d | 0 | 0 | 2 | 0 | 2 | 4 | 0 | 0 | 0 | 0 | 0 |
| LSr.vl | 0 | 0 | 2 | 0 | 0 | 0 | 0 | 3 | 0 | 1 | 1 |
| LSr.dl | 0 | 0 | 2 | 0 | 2 | 4 | 1 | 0 | 0 | 0 | 0 |
| LSc.v | 0 | 0 | 2 | 0 | 3 | 5 | 2 | 1 | 0 | 0 | 0 |
| LSc.d | 0 | 0 | 2 | 1 | 2 | 6 | 0 | 0 | 0 | 0 | 0 |
| LSv | 0 | 0 | 1 | 0 | 0 | 2 | 1 | 2 | 0 | 1 | 1 |
| SH | 0 | 0 | 3 | 3 | 1 | 7 | 0 | 0 | 0 | 0 | 0 |
| SF | 0 | 0 | 1 | 0 | 6 | 6 | 2 | 0 | 0 | 0 | 0 |
| FS | 0 | 6 | 6 | 2 | 0 | 0 | 0 | 4 | 4 | 2 | 2 |
| CP | 4 | 6 | 0 | 0 | 0 | 0 | 0 | 0 | 0 | 0 | 0 |
| AAA | 0 | 4 | 4 | 4 | 1 | 4 | 0 | 4 | 1 | 2 | 0 |
| CEAm | 0 | 0 | 3 | 0 | 0 | 0 | 0 | 5 | 6 | 2 | 3 |
| CEAl | 0 | 0 | 3 | 0 | 0 | 0 | 0 | 3 | 6 | 0 | 2 |
| CEAc | 0 | 0 | 3 | 0 | 0 | 1 | 0 | 4 | 4 | 3 | 4 |
| IA | 0 | 0 | 4 | 0 | 4 | 4 | 0 | 0 | 0 | 0 | 0 |
| MEAav | 0 | 0 | 3 | 0 | 0 | 1 | 0 | 3 | 0 | 2 | 0 |
| MEAad | 0 | 0 | 4 | 1 | 1 | 4 | 0 | 6 | 0 | 4 | 3 |
| MEApv | 0 | 0 | 3 | 1 | 0 | 1 | 0 | 5 | 0 | 4 | 4 |
| MEApd | 0 | 0 | 2 | 0 | 0 | 0 | 0 | 2 | 0 | 2 | 1 |
| BA | 0 | 0 | 0 | 0 | 0 | 0 | 0 | 0 | 0 | 0 | 0 |

TABLE 5B
Data matrices for the association and commissural connections of the rat cerebral
nuclei derived from collation of connection reports from the primary literature (continued)

| | BSTdm | BSTal | BSTov | BSTju | BSTrh | BSTpr | BSTIf | BSTtr | BSTd | BSTse | BAC |
|---|---|---|---|---|---|---|---|---|---|---|---|
| GPm | 0 | 0 | 0 | 0 | 0 | 0 | 0 | 0 | 0 | 0 | 0 |
| GPl | 0 | 0 | 0 | 0 | 0 | 0 | 0 | 0 | 0 | 0 | 0 |
| SI | 1 | 3 | 0 | 0 | 3 | 2 | 0 | 0 | 1 | 0 | 0 |
| MA | 0 | 0 | 0 | 0 | 0 | 0 | 0 | 0 | 0 | 0 | 0 |
| MS | 2 | 0 | 0 | 0 | 0 | 0 | 0 | 0 | 0 | 0 | 0 |
| NDB | 0 | 3 | 0 | 0 | 0 | 0 | 0 | 0 | 0 | 0 | 0 |
| TRS | 0 | 0 | 0 | 0 | 0 | 0 | 0 | 0 | 0 | 0 | 0 |
| BSTam | 4 | 6 | 2 | 0 | 4 | 2 | 4 | 4 | 0 | 2 | 0 |
| BSTfu | 4 | 5 | 4 | 2 | 4 | 2 | 2 | 2 | 0 | 2 | 0 |
| BSTv | 5 | 3 | 1 | 0 | 3 | 0 | 0 | 1 | 0 | 0 | 0 |
| BSTmg | 6 | 3 | 2 | 0 | 6 | 1 | 1 | 0 | 0 | 0 | 0 |
| BSTdm | 0 | 5 | 2 | 0 | 4 | 2 | 4 | 3 | 1 | 0 | 0 |
| BSTal | 3 | 0 | 4 | 4 | 6 | 1 | 2 | 2 | 0 | 0 | 0 |
| BSTov | 2 | 4 | 0 | 4 | 4 | 0 | 3 | 3 | 0 | 0 | 0 |
| BSTju | 1 | 3 | 1 | 0 | 1 | 0 | 2 | 2 | 0 | 6 | 0 |
| BSTrh | 4 | 7 | 6 | 3 | 0 | 0 | 4 | 4 | 0 | 0 | 0 |
| BSTpr | 2 | 2 | 0 | 0 | 0 | 0 | 3 | 2 | 2 | 0 | 0 |
| BSTif | 1 | 1 | 0 | 0 | 0 | 2 | 0 | 4 | 2 | 1 | 0 |
| BSTtr | 2 | 2 | 2 | 0 | 3 | 2 | 4 | 0 | 1 | 0 | 0 |
| BSTd | 4 | 6 | 2 | 2 | 2 | 2 | 7 | 7 | 0 | 6 | 0 |
| BSTsc | 0 | 0 | 0 | 0 | 0 | 0 | 0 | 0 | 0 | 0 | 0 |
| BAC | 0 | 0 | 0 | 0 | 0 | 0 | 0 | 0 | 0 | 0 | 0 |
| OT | 0 | 0 | 0 | 0 | 0 | 0 | 0 | 0 | 0 | 0 | 0 |
| ACB | 0 | 0 | 0 | 0 | 0 | 0 | 0 | 0 | 0 | 0 | 0 |
| LSr.m.v | 0 | 0 | 0 | 0 | 0 | 0 | 1 | 0 | 0 | 0 | 0 |
| LSr.m.d | 0 | 1 | 0 | 0 | 0 | 0 | 0 | 0 | 0 | 0 | 0 |
| LSr.vl | 1 | 2 | 0 | 0 | 0 | 2 | 0 | 0 | 0 | 0 | 2 |
| LSr.dl | 0 | 0 | 0 | 0 | 0 | 0 | 0 | 0 | 0 | 0 | 0 |
| LSc.v | 0 | 0 | 0 | 0 | 0 | 0 | 0 | 0 | 0 | 0 | 0 |
| LSc.d | 0 | 0 | 0 | 0 | 0 | 0 | 0 | 0 | 0 | 0 | 0 |
| LSv | 1 | 0 | 0 | 0 | 0 | 2 | 1 | 1 | 3 | 0 | 0 |
| SH | 0 | 1 | 0 | 0 | 0 | 0 | 0 | 0 | 0 | 0 | 0 |
| SF | 0 | 0 | 0 | 0 | 0 | 0 | 0 | 0 | 0 | 0 | 0 |
| FS | 2 | 3 | 0 | 0 | 2 | 2 | 0 | 0 | 0 | 0 | 0 |
| CP | 0 | 0 | 0 | 0 | 0 | 0 | 0 | 0 | 0 | 0 | 0 |
| AAA | 1 | 4 | 2 | 0 | 6 | 2 | 3 | 6 | 0 | 0 | 0 |
| CEAm | 2 | 6 | 6 | 1 | 7 | 1 | 3 | 5 | 2 | 0 | 0 |
| CEAl | 1 | 4 | 5 | 0 | 2 | 0 | 1 | 2 | 0 | 0 | 0 |
| CEAc | 2 | 7 | 2 | 1 | 6 | 1 | 2 | 3 | 2 | 0 | 0 |
| IA | 0 | 0 | 0 | 0 | 0 | 0 | 0 | 0 | 0 | 0 | 0 |
| MEAav | 0 | 4 | 0 | 0 | 0 | 2 | 3 | 3 | 0 | 6 | 0 |
| MEAad | 4 | 3 | 1 | 0 | 4 | 2 | 6 | 6 | 2 | 2 | 0 |
| MEApv | 2 | 2 | 1 | 0 | 0 | 3 | 6 | 7 | 2 | 0 | 0 |
| MEApd | 1 | 1 | 0 | 0 | 0 | 7 | 4 | 3 | 2 | 0 | 0 |
| BA | 0 | 0 | 0 | 0 | 0 | 0 | 0 | 0 | 0 | 0 | 0 |

TABLE 5C
Data matrices for the association and commissural connections of the rat cerebral
nuclei derived from collation of connection reports from the primary literature (continued)

| | OT | ACB | LSr.m.v | LSr.m.d | LSr.vl | LSr.dl | LSc.v | LSc.d | LSv | SH | SF |
|---|---|---|---|---|---|---|---|---|---|---|---|
| GPm | 0 | 0 | 0 | 0 | 0 | 0 | 0 | 0 | 0 | 0 | 0 |
| GPl | 0 | 0 | 0 | 0 | 0 | 0 | 0 | 0 | 0 | 0 | 0 |
| SI | 0 | 6 | 3 | 4 | 0 | 0 | 0 | 0 | 2 | 0 | 1 |
| MA | 0 | 0 | 0 | 0 | 0 | 0 | 0 | 0 | 0 | 0 | 0 |
| MS | 2 | 2 | 2 | 2 | 2 | 2 | 0 | 2 | 2 | 2 | 2 |
| NDB | 2 | 0 | 0 | 3 | 0 | 3 | 3 | 1 | 0 | 2 | 2 |
| TRS | 0 | 0 | 0 | 0 | 0 | 0 | 0 | 0 | 0 | 0 | 0 |
| BSTam | 3 | 7 | 3 | 0 | 3 | 2 | 2 | 1 | 3 | 0 | 1 |
| BSTfu | 0 | 4 | 2 | 0 | 2 | 1 | 0 | 1 | 2 | 0 | 1 |
| BSTv | 0 | 3 | 2 | 1 | 1 | 1 | 1 | 1 | 1 | 0 | 1 |
| BSTmg | 0 | 3 | 1 | 0 | 1 | 1 | 0 | 0 | 0 | 0 | 1 |
| BSTdm | 1 | 3 | 3 | 1 | 2 | 3 | 2 | 1 | 1 | 0 | 0 |
| BSTal | 1 | 2 | 2 | 1 | 2 | 2 | 1 | 1 | 0 | 0 | 0 |
| BSTov | 0 | 3 | 0 | 0 | 0 | 0 | 0 | 0 | 0 | 0 | 0 |
| BSTju | 0 | 2 | 0 | 0 | 0 | 1 | 0 | 0 | 0 | 0 | 1 |
| BSTrh | 3 | 5 | 2 | 0 | 2 | 2 | 1 | 1 | 2 | 0 | 1 |
| BSTpr | 0 | 1 | 1 | 0 | 3 | 2 | 2 | 0 | 3 | 0 | 0 |
| BSTif | 2 | 1 | 3 | 2 | 3 | 3 | 2 | 0 | 2 | 0 | 1 |
| BSTtr | 1 | 2 | 3 | 1 | 3 | 2 | 2 | 0 | 1 | 0 | 0 |
| BSTd | 4 | 4 | 0 | 0 | 5 | 0 | 0 | 0 | 6 | 0 | 0 |
| BSTse | 0 | 0 | 0 | 0 | 0 | 0 | 0 | 0 | 0 | 0 | 0 |
| BAC | 0 | 0 | 0 | 0 | 0 | 0 | 0 | 0 | 0 | 0 | 0 |
| OT | 0 | 0 | 0 | 0 | 0 | 0 | 0 | 0 | 0 | 0 | 0 |
| ACB | 0 | 0 | 0 | 0 | 0 | 0 | 0 | 0 | 0 | 0 | 0 |
| LSr.m.v | 0 | 0 | 0 | 1 | 1 | 0 | 0 | 0 | 0 | 0 | 4 |
| LSr.m.d | 0 | 0 | 3 | 0 | 0 | 0 | 1 | 3 | 0 | 0 | 2 |
| LSr.vl | 0 | 4 | 4 | 0 | 0 | 2 | 2 | 0 | 2 | 0 | 2 |
| LSr.dl | 0 | 0 | 3 | 2 | 2 | 0 | 3 | 2 | 0 | 0 | 3 |
| LSc.v | 0 | 0 | 4 | 0 | 0 | 0 | 0 | 1 | 0 | 0 | 3 |
| LSc.d | 0 | 0 | 4 | 4 | 0 | 4 | 0 | 0 | 0 | 0 | 0 |
| LSv | 0 | 0 | 1 | 0 | 2 | 0 | 2 | 0 | 0 | 0 | 1 |
| SH | 0 | 0 | 0 | 3 | 3 | 0 | 0 | 4 | 0 | 0 | 0 |
| SF | 0 | 0 | 2 | 3 | 0 | 2 | 0 | 0 | 0 | 0 | 0 |
| FS | 2 | 2 | 0 | 0 | 0 | 1 | 1 | 0 | 0 | 0 | 0 |
| CP | 0 | 0 | 0 | 0 | 0 | 0 | 0 | 0 | 0 | 0 | 0 |
| AAA | 4 | 4 | 1 | 0 | 0 | 0 | 0 | 0 | 0 | 0 | 0 |
| CEAm | 0 | 0 | 0 | 0 | 0 | 0 | 0 | 0 | 0 | 0 | 0 |
| CEAl | 0 | 0 | 0 | 0 | 0 | 0 | 0 | 0 | 0 | 0 | 0 |
| CEAc | 0 | 0 | 1 | 0 | 0 | 0 | 0 | 0 | 0 | 0 | 0 |
| IA | 4 | 4 | 0 | 0 | 0 | 0 | 0 | 0 | 0 | 0 | 0 |
| MEAav | 2 | 2 | 2 | 0 | 0 | 0 | 0 | 0 | 0 | 0 | 0 |
| MEAad | 2 | 2 | 4 | 1 | 5 | 2 | 2 | 2 | 3 | 0 | 1 |
| MEApv | 2 | 0 | 2 | 0 | 3 | 2 | 2 | 0 | 3 | 0 | 0 |
| MEApd | 0 | 0 | 1 | 0 | 3 | 2 | 2 | 0 | 3 | 0 | 1 |
| BA | 0 | 0 | 0 | 0 | 0 | 0 | 0 | 0 | 0 | 0 | 0 |

TABLE 5D
Data matrices for the association and commissural connections of the rat cerebral
nuclei derived from collation of connection reports from the primary literature (continued)

| | FS | CP | AAA | CEAm | CEAl | CEAc | IA | MEAAv | MEAad | MEApv | MEApd | BA |
|---|---|---|---|---|---|---|---|---|---|---|---|---|
| GPm | 0 | 4 | 0 | 0 | 0 | 0 | 0 | 0 | 0 | 0 | 0 | 0 |
| GPl | 0 | 4 | 0 | 0 | 2 | 0 | 0 | 0 | 0 | 0 | 0 | 0 |
| SI | 3 | 2 | 2 | 4 | 2 | 1 | 2 | 2 | 4 | 3 | 4 | 1 |
| MA | 0 | 0 | 3 | 2 | 0 | 0 | 0 | 0 | 2 | 2 | 3 | 0 |
| MS | 0 | 0 | 0 | 0 | 0 | 2 | 0 | 0 | 0 | 0 | 3 | 0 |
| NDB | 0 | 0 | 0 | 0 | 2 | 3 | 0 | 3 | 3 | 3 | 3 | 0 |
| TRS | 0 | 0 | 0 | 0 | 0 | 0 | 0 | 0 | 0 | 0 | 0 | 0 |
| BSTam | 2 | 0 | 1 | 4 | 2 | 2 | 0 | 0 | 2 | 1 | 1 | 0 |
| BSTfu | 1 | 0 | 0 | 6 | 1 | 1 | 1 | 0 | 0 | 0 | 0 | 0 |
| BSTv | 0 | 0 | 0 | 4 | 1 | 3 | 1 | 0 | 2 | 0 | 0 | 0 |
| BSTmg | 1 | 0 | 1 | 4 | 0 | 2 | 0 | 0 | 0 | 0 | 0 | 0 |
| BSTdm | 2 | 0 | 2 | 5 | 1 | 2 | 0 | 0 | 3 | 2 | 2 | 0 |
| BSTal | 2 | 0 | 2 | 7 | 2 | 2 | 2 | 0 | 2 | 0 | 2 | 0 |
| BSTov | 1 | 1 | 0 | 5 | 3 | 2 | 1 | 0 | 0 | 0 | 0 | 0 |
| BSTju | 1 | 3 | 1 | 4 | 2 | 2 | 0 | 0 | 1 | 0 | 0 | 0 |
| BSTrh | 4 | 1 | 2 | 7 | 4 | 7 | 3 | 1 | 2 | 1 | 1 | 1 |
| BSTpr | 0 | 0 | 1 | 1 | 0 | 1 | 1 | 0 | 2 | 1 | 6 | 0 |
| BSTif | 0 | 0 | 2 | 2 | 0 | 2 | 1 | 2 | 4 | 4 | 3 | 0 |
| BSTtr | 2 | 0 | 4 | 5 | 0 | 3 | 3 | 2 | 3 | 2 | 2 | 0 |
| BSTd | 0 | 0 | 0 | 4 | 0 | 0 | 0 | 0 | 6 | 2 | 6 | 0 |
| BSTse | 0 | 0 | 0 | 0 | 0 | 0 | 0 | 0 | 0 | 0 | 0 | 0 |
| BAC | 0 | 0 | 0 | 0 | 0 | 0 | 0 | 0 | 0 | 0 | 0 | 0 |
| OT | 0 | 0 | 0 | 0 | 0 | 0 | 0 | 0 | 0 | 0 | 0 | 0 |
| ACB | 0 | 3 | 0 | 1 | 0 | 0 | 0 | 0 | 0 | 0 | 0 | 0 |
| LSr.m.v | 0 | 0 | 0 | 0 | 0 | 0 | 0 | 0 | 0 | 0 | 0 | 0 |
| LSr.m.d | 0 | 0 | 0 | 0 | 0 | 0 | 0 | 0 | 0 | 0 | 0 | 0 |
| LSr.vl | 2 | 0 | 0 | 0 | 0 | 0 | 0 | 0 | 0 | 0 | 0 | 0 |
| LSr.dl | 0 | 0 | 0 | 0 | 0 | 0 | 0 | 0 | 2 | 0 | 0 | 0 |
| LSc.v | 0 | 0 | 0 | 0 | 0 | 0 | 0 | 0 | 0 | 0 | 0 | 0 |
| LSc.d | 0 | 0 | 0 | 0 | 0 | 0 | 0 | 0 | 1 | 0 | 0 | 0 |
| LSv | 0 | 0 | 0 | 0 | 0 | 0 | 0 | 0 | 0 | 0 | 2 | 0 |
| SH | 4 | 0 | 2 | 2 | 0 | 0 | 0 | 0 | 0 | 0 | 0 | 0 |
| SF | 0 | 0 | 0 | 0 | 0 | 0 | 0 | 0 | 0 | 0 | 0 | 0 |
| FS | 0 | 2 | 2 | 6 | 1 | 4 | 2 | 0 | 2 | 0 | 1 | 0 |
| CP | 0 | 0 | 0 | 0 | 0 | 0 | 0 | 0 | 0 | 0 | 0 | 0 |
| AAA | 6 | 0 | 0 | 2 | 2 | 2 | 0 | 0 | 0 | 0 | 0 | 0 |
| CEAm | 2 | 0 | 1 | 0 | 1 | 2 | 0 | 0 | 0 | 0 | 0 | 0 |
| CEAl | 0 | 0 | 0 | 4 | 0 | 2 | 0 | 0 | 0 | 0 | 0 | 0 |
| CEAc | 3 | 0 | 1 | 2 | 4 | 0 | 0 | 0 | 0 | 0 | 0 | 0 |
| IA | 4 | 0 | 0 | 0 | 0 | 0 | 0 | 0 | 0 | 0 | 0 | 0 |
| MEAav | 2 | 0 | 2 | 2 | 2 | 3 | 1 | 0 | 3 | 3 | 3 | 0 |
| MEAad | 3 | 0 | 7 | 4 | 1 | 6 | 6 | 6 | 0 | 6 | 4 | 7 |
| MEApv | 2 | 0 | 3 | 2 | 0 | 3 | 3 | 7 | 6 | 0 | 4 | 7 |
| MEApd | 0 | 0 | 2 | 2 | 0 | 2 | 1 | 4 | 4 | 4 | 0 | 3 |
| BA | 0 | 0 | 0 | 0 | 0 | 0 | 0 | 0 | 0 | 0 | 0 | 0 |

TABLE 6A
Data matrices for the association and commissural connections of the rat cerebral
nuclei derived from collation of connection reports from the primary literature

| | GPm | GPl | SI | MA | MS | NDB | TRS | BSTam | BSTfu | BSTv | BSTmg |
|---|---|---|---|---|---|---|---|---|---|---|---|
| GPm | 0 | 0 | 0 | 0 | 0 | 0 | 0 | 0 | 0 | 0 | 0 |
| GPl | 0 | 0 | 0 | 0 | 0 | 0 | 0 | 0 | 0 | 0 | 0 |
| SI | 0 | 0 | 2 | 0 | 0 | 0 | 0 | 0 | 0 | 0 | 0 |
| MA | 0 | 0 | 0 | 2 | 0 | 0 | 0 | 0 | 0 | 0 | 0 |
| MS | 0 | 0 | 0 | 0 | 2 | 0 | 0 | 0 | 0 | 0 | 0 |
| NDB | 0 | 0 | 0 | 0 | 1 | 0 | 0 | 0 | 0 | 0 | 0 |
| TRS | 0 | 0 | 0 | 0 | 0 | 0 | 0 | 0 | 0 | 0 | 0 |
| BSTam | 0 | 0 | 1 | 0 | 0 | 1 | 0 | 2 | 1 | 0 | 0 |
| BSTfu | 0 | 0 | 1 | 0 | 0 | 0 | 0 | 2 | 1 | 0 | 0 |
| BSTv | 0 | 0 | 0 | 0 | 0 | 0 | 0 | 1 | 1 | 1 | 0 |
| BSTmg | 0 | 0 | 0 | 0 | 0 | 0 | 0 | 2 | 0 | 0 | 0 |
| BSTdm | 0 | 0 | 2 | 0 | 0 | 0 | 0 | 2 | 0 | 1 | 1 |
| BSTal | 0 | 0 | 1 | 0 | 0 | 0 | 0 | 1 | 2 | 1 | 1 |
| BSTov | 0 | 0 | 0 | 0 | 0 | 0 | 0 | 0 | 0 | 0 | 0 |
| BSTju | 0 | 0 | 0 | 0 | 0 | 0 | 0 | 0 | 0 | 0 | 0 |
| BSTrh | 0 | 0 | 2 | 0 | 0 | 0 | 0 | 2 | 1 | 0 | 0 |
| BSTpr | 0 | 0 | 0 | 0 | 0 | 0 | 0 | 0 | 0 | 1 | 0 |
| BSTif | 0 | 0 | 0 | 0 | 0 | 0 | 0 | 1 | 0 | 0 | 1 |
| BSTtr | 0 | 0 | 0 | 0 | 0 | 0 | 0 | 1 | 0 | 0 | 0 |
| BSTd | 0 | 0 | 0 | 0 | 0 | 0 | 0 | 0 | 0 | 0 | 0 |
| BSTse | 0 | 0 | 0 | 0 | 0 | 0 | 0 | 0 | 0 | 0 | 0 |
| BAC | 0 | 0 | 0 | 0 | 0 | 0 | 0 | 0 | 0 | 0 | 0 |
| OT | 0 | 0 | 0 | 0 | 0 | 0 | 0 | 0 | 0 | 0 | 0 |
| ACB | 0 | 0 | 0 | 0 | 0 | 0 | 0 | 0 | 0 | 0 | 0 |
| LSr.m.v | 0 | 0 | 0 | 0 | 0 | 0 | 0 | 0 | 0 | 0 | 0 |
| LSr.m.d | 0 | 0 | 0 | 0 | 0 | 0 | 0 | 0 | 0 | 0 | 0 |
| LSr.vl | 0 | 0 | 0 | 0 | 0 | 0 | 0 | 0 | 0 | 0 | 0 |
| LSr.dl | 0 | 0 | 0 | 0 | 0 | 1 | 0 | 0 | 0 | 0 | 0 |
| LSc.v | 0 | 0 | 0 | 0 | 0 | 0 | 1 | 0 | 0 | 0 | 0 |
| LSc.d | 0 | 0 | 0 | 0 | 0 | 0 | 0 | 0 | 0 | 0 | 0 |
| LSv | 0 | 0 | 0 | 0 | 0 | 0 | 0 | 0 | 0 | 0 | 0 |
| SH | 0 | 0 | 0 | 0 | 0 | 0 | 0 | 0 | 0 | 0 | 0 |
| SF | 0 | 0 | 0 | 0 | 0 | 0 | 0 | 0 | 0 | 0 | 0 |
| FS | 0 | 0 | 0 | 0 | 0 | 0 | 0 | 0 | 0 | 0 | 0 |
| CP | 0 | 0 | 0 | 0 | 0 | 0 | 0 | 0 | 0 | 0 | 0 |
| AAA | 0 | 0 | 0 | 0 | 0 | 0 | 0 | 0 | 0 | 0 | 0 |
| CEAm | 0 | 0 | 0 | 0 | 0 | 0 | 0 | 0 | 0 | 0 | 0 |
| CEAl | 0 | 0 | 0 | 0 | 0 | 0 | 0 | 0 | 0 | 0 | 0 |
| CEAc | 0 | 0 | 0 | 0 | 0 | 0 | 0 | 0 | 0 | 0 | 0 |
| IA | 0 | 0 | 0 | 0 | 0 | 0 | 0 | 0 | 0 | 0 | 0 |
| MEAav | 0 | 0 | 0 | 0 | 0 | 0 | 0 | 0 | 0 | 0 | 0 |
| MEAad | 0 | 0 | 0 | 0 | 0 | 0 | 0 | 0 | 0 | 0 | 0 |
| MEApv | 0 | 0 | 0 | 0 | 0 | 0 | 0 | 0 | 0 | 0 | 0 |
| MEApd | 0 | 0 | 0 | 0 | 0 | 0 | 0 | 0 | 0 | 0 | 0 |
| BA | 0 | 0 | 0 | 0 | 0 | 0 | 0 | 0 | 0 | 0 | 0 |

TABLE 6B
Data matrices for the association and commissural connections of the rat cerebral
nuclei derived from collation of connection reports from the primary literature (continued)

| | BSTdm | BSTal | BSTov | BSTju | BSTrh | BSTpr | BSTIf | BSTtr | BSTd | BSTse | BAC |
|---|---|---|---|---|---|---|---|---|---|---|---|
| GPm | 0 | 0 | 0 | 0 | 0 | 0 | 0 | 0 | 0 | 0 | 0 |
| GPl | 0 | 0 | 0 | 0 | 0 | 0 | 0 | 0 | 0 | 0 | 0 |
| SI | 0 | 0 | 0 | 0 | 0 | 0 | 0 | 0 | 0 | 0 | 0 |
| MA | 0 | 0 | 0 | 0 | 0 | 0 | 0 | 0 | 0 | 0 | 0 |
| MS | 0 | 0 | 0 | 0 | 0 | 0 | 0 | 0 | 0 | 0 | 0 |
| NDB | 0 | 0 | 0 | 0 | 0 | 0 | 0 | 0 | 0 | 0 | 0 |
| TRS | 0 | 0 | 0 | 0 | 0 | 0 | 0 | 0 | 0 | 0 | 0 |
| BSTam | 1 | 1 | 0 | 0 | 0 | 1 | 0 | 0 | 0 | 0 | 0 |
| BSTfu | 1 | 1 | 1 | 0 | 0 | 0 | 0 | 0 | 0 | 0 | 0 |
| BSTv | 1 | 0 | 0 | 0 | 0 | 0 | 0 | 0 | 0 | 0 | 0 |
| BSTmg | 1 | 0 | 0 | 0 | 1 | 0 | 0 | 0 | 0 | 0 | 0 |
| BSTdm | 1 | 1 | 0 | 0 | 0 | 1 | 2 | 1 | 1 | 0 | 0 |
| BSTal | 1 | 2 | 0 | 0 | 1 | 0 | 0 | 1 | 0 | 0 | 0 |
| BSTov | 0 | 0 | 0 | 0 | 0 | 0 | 0 | 0 | 0 | 0 | 0 |
| BSTju | 0 | 0 | 0 | 0 | 0 | 0 | 0 | 0 | 0 | 0 | 0 |
| BSTrh | 2 | 1 | 1 | 0 | 1 | 0 | 2 | 0 | 0 | 0 | 0 |
| BSTpr | 0 | 0 | 0 | 0 | 0 | 0 | 0 | 0 | 0 | 0 | 0 |
| BSTif | 0 | 0 | 0 | 0 | 0 | 0 | 1 | 0 | 0 | 0 | 0 |
| BSTtr | 0 | 0 | 0 | 0 | 0 | 0 | 0 | 0 | 0 | 0 | 0 |
| BSTd | 0 | 0 | 0 | 0 | 0 | 0 | 0 | 0 | 0 | 0 | 0 |
| BSTse | 0 | 0 | 0 | 0 | 0 | 0 | 0 | 0 | 0 | 0 | 0 |
| BAC | 0 | 0 | 0 | 0 | 0 | 0 | 0 | 0 | 0 | 0 | 0 |
| OT | 0 | 0 | 0 | 0 | 0 | 0 | 0 | 0 | 0 | 0 | 0 |
| ACB | 0 | 0 | 0 | 0 | 0 | 0 | 0 | 0 | 0 | 0 | 0 |
| LSr.m.v | 0 | 0 | 0 | 0 | 0 | 0 | 0 | 0 | 0 | 0 | 0 |
| LSr.m.d | 0 | 0 | 0 | 0 | 0 | 0 | 0 | 0 | 0 | 0 | 0 |
| LSr.vl | 0 | 0 | 0 | 0 | 0 | 0 | 0 | 0 | 0 | 0 | 0 |
| LSr.dl | 0 | 0 | 0 | 0 | 0 | 0 | 0 | 0 | 0 | 0 | 0 |
| LSc.v | 0 | 0 | 0 | 0 | 0 | 0 | 0 | 0 | 0 | 0 | 0 |
| LSc.d | 0 | 0 | 0 | 0 | 0 | 0 | 0 | 0 | 0 | 0 | 0 |
| LSv | 0 | 0 | 0 | 0 | 0 | 0 | 0 | 0 | 0 | 0 | 0 |
| SH | 0 | 0 | 0 | 0 | 0 | 0 | 0 | 0 | 0 | 0 | 0 |
| SF | 0 | 0 | 0 | 0 | 0 | 0 | 0 | 0 | 0 | 0 | 0 |
| FS | 0 | 0 | 0 | 0 | 0 | 0 | 0 | 0 | 0 | 0 | 0 |
| CP | 0 | 0 | 0 | 0 | 0 | 0 | 0 | 0 | 0 | 0 | 0 |
| AAA | 0 | 0 | 0 | 0 | 0 | 0 | 0 | 0 | 0 | 0 | 0 |
| CEAm | 0 | 0 | 0 | 0 | 0 | 0 | 0 | 0 | 0 | 0 | 0 |
| CEAl | 0 | 0 | 0 | 0 | 0 | 0 | 0 | 0 | 0 | 0 | 0 |
| CEAc | 0 | 0 | 0 | 0 | 0 | 0 | 0 | 0 | 0 | 0 | 0 |
| IA | 0 | 0 | 0 | 0 | 0 | 0 | 0 | 0 | 0 | 0 | 0 |
| MEAav | 0 | 0 | 0 | 0 | 0 | 0 | 0 | 0 | 0 | 0 | 0 |
| MEAad | 0 | 0 | 0 | 0 | 0 | 0 | 0 | 0 | 0 | 0 | 0 |
| MEApv | 0 | 0 | 0 | 0 | 0 | 0 | 0 | 0 | 0 | 0 | 0 |
| MEApd | 0 | 0 | 0 | 0 | 0 | 0 | 0 | 0 | 0 | 0 | 0 |
| BA | 0 | 0 | 0 | 0 | 0 | 0 | 0 | 0 | 0 | 0 | 0 |

TABLE 6C
Data matrices for the association and commissural connections of the rat cerebral
nuclei derived from collation of connection reports from the primary literature (continued)

| | OT | ACB | LSr.m.v | LSr.m.d | LSr.vl | LSr.dl | LSc.v | LSc.d | LSv | SH | SF |
|---|---|---|---|---|---|---|---|---|---|---|---|
| GPm | 0 | 0 | 0 | 0 | 0 | 0 | 0 | 0 | 0 | 0 | 0 |
| GPl | 0 | 0 | 0 | 0 | 0 | 0 | 0 | 0 | 0 | 0 | 0 |
| SI | 0 | 0 | 0 | 0 | 0 | 0 | 0 | 0 | 0 | 0 | 0 |
| MA | 0 | 0 | 0 | 0 | 0 | 0 | 0 | 0 | 0 | 0 | 0 |
| MS | 0 | 0 | 2 | 2 | 0 | 0 | 0 | 2 | 0 | 0 | 0 |
| NDB | 0 | 0 | 0 | 3 | 0 | 3 | 2 | 0 | 0 | 0 | 0 |
| TRS | 0 | 0 | 0 | 0 | 0 | 0 | 0 | 0 | 0 | 0 | 0 |
| BSTam | 0 | 1 | 1 | 0 | 1 | 1 | 0 | 1 | 1 | 0 | 0 |
| BSTfu | 0 | 1 | 0 | 0 | 0 | 0 | 0 | 0 | 0 | 0 | 0 |
| BSTv | 0 | 0 | 1 | 1 | 1 | 0 | 0 | 0 | 0 | 0 | 0 |
| BSTmg | 0 | 0 | 0 | 0 | 1 | 0 | 0 | 0 | 0 | 0 | 0 |
| BSTdm | 0 | 0 | 1 | 0 | 0 | 0 | 0 | 0 | 0 | 0 | 0 |
| BSTal | 0 | 0 | 1 | 0 | 0 | 0 | 1 | 1 | 0 | 0 | 0 |
| BSTov | 0 | 0 | 0 | 0 | 0 | 0 | 0 | 0 | 0 | 0 | 0 |
| BSTju | 0 | 0 | 0 | 0 | 0 | 0 | 0 | 0 | 0 | 0 | 0 |
| BSTrh | 0 | 0 | 0 | 0 | 0 | 0 | 0 | 0 | 0 | 0 | 0 |
| BSTpr | 0 | 0 | 0 | 0 | 0 | 0 | 0 | 0 | 0 | 0 | 0 |
| BSTif | 0 | 0 | 0 | 0 | 0 | 2 | 0 | 0 | 0 | 0 | 0 |
| BSTtr | 0 | 0 | 1 | 1 | 0 | 0 | 0 | 0 | 0 | 0 | 0 |
| BSTd | 0 | 0 | 0 | 0 | 0 | 0 | 0 | 0 | 0 | 0 | 0 |
| BSTse | 0 | 0 | 0 | 0 | 0 | 0 | 0 | 0 | 0 | 0 | 0 |
| BAC | 0 | 0 | 0 | 0 | 0 | 0 | 0 | 0 | 0 | 0 | 0 |
| OT | 0 | 0 | 0 | 0 | 0 | 0 | 0 | 0 | 0 | 0 | 0 |
| ACB | 0 | 0 | 0 | 0 | 0 | 0 | 0 | 0 | 0 | 0 | 0 |
| LSr.m.v | 0 | 0 | 0 | 0 | 0 | 0 | 0 | 0 | 0 | 0 | 0 |
| LSr.m.d | 0 | 0 | 0 | 0 | 0 | 0 | 0 | 0 | 0 | 0 | 0 |
| LSr.vl | 0 | 0 | 1 | 0 | 1 | 0 | 0 | 0 | 0 | 0 | 1 |
| LSr.dl | 0 | 0 | 1 | 0 | 0 | 0 | 0 | 0 | 0 | 0 | 1 |
| LSc.v | 0 | 0 | 0 | 0 | 0 | 0 | 0 | 0 | 0 | 0 | 1 |
| LSc.d | 0 | 0 | 0 | 0 | 0 | 0 | 0 | 1 | 0 | 0 | 0 |
| LSv | 0 | 0 | 0 | 0 | 0 | 0 | 0 | 0 | 0 | 0 | 0 |
| SH | 0 | 0 | 0 | 0 | 0 | 0 | 0 | 0 | 0 | 0 | 0 |
| SF | 0 | 0 | 0 | 0 | 0 | 0 | 0 | 0 | 0 | 0 | 0 |
| FS | 0 | 0 | 0 | 0 | 0 | 0 | 0 | 0 | 0 | 0 | 0 |
| CP | 0 | 0 | 0 | 0 | 0 | 0 | 0 | 0 | 0 | 0 | 0 |
| AAA | 0 | 0 | 0 | 0 | 0 | 0 | 0 | 0 | 0 | 0 | 0 |
| CEAm | 0 | 0 | 0 | 0 | 0 | 0 | 0 | 0 | 0 | 0 | 0 |
| CEAl | 0 | 0 | 0 | 0 | 0 | 0 | 0 | 0 | 0 | 0 | 0 |
| CEAc | 0 | 0 | 0 | 0 | 0 | 0 | 0 | 0 | 0 | 0 | 0 |
| IA | 0 | 0 | 0 | 0 | 0 | 0 | 0 | 0 | 0 | 0 | 0 |
| MEAav | 0 | 0 | 0 | 0 | 0 | 0 | 0 | 0 | 0 | 0 | 0 |
| MEAad | 0 | 0 | 0 | 0 | 0 | 0 | 0 | 0 | 0 | 0 | 0 |
| MEApv | 0 | 0 | 0 | 0 | 0 | 0 | 0 | 0 | 0 | 0 | 0 |
| MEApd | 0 | 0 | 0 | 0 | 0 | 0 | 0 | 0 | 0 | 0 | 0 |
| BA | 0 | 0 | 0 | 0 | 0 | 0 | 0 | 0 | 0 | 0 | 0 |

TABLE 6D
Data matrices for the association and commissural connections of the rat cerebral
nuclei derived from collation of connection reports from the primary literature (continued)

| | FS | CP | AAA | CEAm | CEAl | CEAc | IA | MEAav | MEAad | MEApv | MEApd | BA |
|---|---|---|---|---|---|---|---|---|---|---|---|---|
| GPm | 0 | 0 | 0 | 0 | 0 | 0 | 0 | 0 | 0 | 0 | 0 | 0 |
| GPl | 0 | 0 | 0 | 0 | 0 | 0 | 0 | 0 | 0 | 0 | 0 | 0 |
| SI | 0 | 0 | 0 | 0 | 0 | 0 | 0 | 0 | 0 | 0 | 0 | 0 |
| MA | 0 | 0 | 0 | 0 | 0 | 0 | 0 | 0 | 0 | 0 | 0 | 0 |
| MS | 0 | 0 | 0 | 0 | 0 | 0 | 0 | 0 | 0 | 0 | 0 | 0 |
| NDB | 0 | 0 | 0 | 0 | 0 | 0 | 0 | 0 | 0 | 0 | 0 | 0 |
| TRS | 0 | 0 | 0 | 0 | 0 | 0 | 0 | 0 | 0 | 0 | 0 | 0 |
| BSTam | 0 | 0 | 0 | 1 | 0 | 0 | 0 | 0 | 0 | 0 | 0 | 0 |
| BSTfu | 0 | 0 | 0 | 2 | 0 | 0 | 0 | 0 | 0 | 0 | 0 | 0 |
| BSTv | 0 | 0 | 0 | 0 | 0 | 0 | 0 | 0 | 0 | 0 | 0 | 0 |
| BSTmg | 0 | 0 | 0 | 0 | 0 | 0 | 0 | 0 | 0 | 0 | 0 | 0 |
| BSTdm | 0 | 0 | 0 | 2 | 0 | 0 | 0 | 0 | 0 | 0 | 0 | 0 |
| BSTal | 0 | 0 | 0 | 0 | 0 | 0 | 0 | 0 | 0 | 0 | 0 | 0 |
| BSTov | 0 | 0 | 0 | 0 | 0 | 0 | 0 | 0 | 0 | 0 | 0 | 0 |
| BSTju | 0 | 0 | 0 | 0 | 0 | 0 | 0 | 0 | 0 | 0 | 0 | 0 |
| BSTrh | 0 | 0 | 0 | 2 | 1 | 1 | 0 | 0 | 0 | 0 | 0 | 0 |
| BSTpr | 0 | 0 | 0 | 0 | 0 | 0 | 0 | 0 | 0 | 0 | 0 | 0 |
| BSTif | 0 | 0 | 0 | 0 | 0 | 0 | 0 | 0 | 1 | 0 | 0 | 0 |
| BSTtr | 0 | 0 | 0 | 0 | 0 | 0 | 0 | 0 | 0 | 0 | 0 | 0 |
| BSTd | 0 | 0 | 0 | 0 | 0 | 0 | 0 | 0 | 0 | 0 | 0 | 0 |
| BSTse | 0 | 0 | 0 | 0 | 0 | 0 | 0 | 0 | 0 | 0 | 0 | 0 |
| BAC | 0 | 0 | 0 | 0 | 0 | 0 | 0 | 0 | 0 | 0 | 0 | 0 |
| OT | 0 | 0 | 0 | 0 | 0 | 0 | 0 | 0 | 0 | 0 | 0 | 0 |
| ACB | 0 | 0 | 0 | 0 | 0 | 0 | 0 | 0 | 0 | 0 | 0 | 0 |
| LSr.m.v | 0 | 0 | 0 | 0 | 0 | 0 | 0 | 0 | 0 | 0 | 0 | 0 |
| LSr.m.d | 0 | 0 | 0 | 0 | 0 | 0 | 0 | 0 | 0 | 0 | 0 | 0 |
| LSr.vl | 0 | 0 | 0 | 0 | 0 | 0 | 0 | 0 | 0 | 0 | 0 | 0 |
| LSr.dl | 0 | 0 | 0 | 0 | 0 | 0 | 0 | 0 | 0 | 0 | 0 | 0 |
| LSc.v | 0 | 0 | 0 | 0 | 0 | 0 | 0 | 0 | 0 | 0 | 0 | 0 |
| LSc.d | 0 | 0 | 0 | 0 | 0 | 0 | 0 | 0 | 0 | 0 | 0 | 0 |
| LSv | 0 | 0 | 0 | 0 | 0 | 0 | 0 | 0 | 0 | 0 | 0 | 0 |
| SH | 0 | 0 | 0 | 0 | 0 | 0 | 0 | 0 | 0 | 0 | 0 | 0 |
| SF | 0 | 0 | 0 | 0 | 0 | 0 | 0 | 0 | 0 | 0 | 0 | 0 |
| FS | 0 | 0 | 0 | 0 | 0 | 0 | 0 | 0 | 0 | 0 | 0 | 0 |
| CP | 0 | 0 | 0 | 0 | 0 | 0 | 0 | 0 | 0 | 0 | 0 | 0 |
| AAA | 0 | 0 | 0 | 0 | 0 | 0 | 0 | 0 | 0 | 0 | 0 | 0 |
| CEAm | 0 | 0 | 0 | 0 | 0 | 0 | 0 | 0 | 0 | 0 | 0 | 0 |
| CEAl | 0 | 0 | 0 | 0 | 0 | 0 | 0 | 0 | 0 | 0 | 0 | 0 |
| CEAc | 0 | 0 | 0 | 0 | 0 | 0 | 0 | 0 | 0 | 0 | 0 | 0 |
| IA | 0 | 0 | 0 | 0 | 0 | 0 | 0 | 0 | 0 | 0 | 0 | 0 |
| MEAav | 0 | 0 | 0 | 0 | 0 | 0 | 0 | 0 | 0 | 0 | 0 | 0 |
| MEAad | 0 | 0 | 0 | 0 | 0 | 0 | 0 | 0 | 0 | 0 | 0 | 0 |
| MEApv | 0 | 0 | 0 | 0 | 0 | 0 | 0 | 0 | 0 | 0 | 0 | 0 |
| MEApd | 0 | 0 | 0 | 0 | 0 | 0 | 0 | 0 | 0 | 0 | 0 | 0 |
| BA | 0 | 0 | 0 | 0 | 0 | 0 | 0 | 0 | 0 | 0 | 0 | 0 |

TABLE 7A
Data matrices for the association and commissural connections of the rat cerebral
nuclei derived from collation of connection reports from the primary literature

| | GPm | GPl | SI | MA | MS | NDB | TRS | BSTam | BSTfu | BSTv | BSTmg |
|---|---|---|---|---|---|---|---|---|---|---|---|
| GPm | 0 | 6 | 3 | 3 | 3 | 3 | 3 | 3 | 3 | 3 | 3 |
| GPl | 11 | 0 | 6 | 3 | 3 | 3 | 3 | 3 | 3 | 3 | 3 |
| SI | 3 | 6 | 0 | 7 | 3 | 6 | 3 | 7 | 1 | 7 | 7 |
| MA | 3 | 3 | 5 | 0 | 6 | 7 | 3 | 1 | 1 | 1 | 1 |
| MS | 3 | 3 | 4 | 6 | 0 | 10 | 4 | 4 | 3 | 3 | 3 |
| NDB | 3 | 3 | 4 | 6 | 6 | 0 | 3 | 7 | 3 | 3 | 3 |
| TRS | 3 | 3 | 3 | 3 | 3 | 3 | 0 | 3 | 3 | 3 | 3 |
| BSTam | 3 | 3 | 11 | 3 | 3 | 5 | 3 | 0 | 11 | 9 | 9 |
| BSTfu | 3 | 3 | 9 | 3 | 3 | 6 | 3 | 11 | 0 | 9 | 4 |
| BSTv | 3 | 3 | 7 | 3 | 4 | 3 | 3 | 10 | 10 | 0 | 10 |
| BSTmg | 3 | 3 | 7 | 3 | 3 | 3 | 3 | 9 | 11 | 11 | 0 |
| BSTdm | 3 | 3 | 10 | 5 | 5 | 5 | 3 | 9 | 10 | 11 | 11 |
| BSTal | 3 | 5 | 11 | 5 | 3 | 5 | 3 | 11 | 11 | 7 | 9 |
| BSTov | 3 | 5 | 10 | 3 | 3 | 3 | 3 | 7 | 12 | 6 | 6 |
| BSTju | 3 | 5 | 12 | 3 | 3 | 3 | 3 | 5 | 5 | 5 | 5 |
| BSTrh | 3 | 6 | 12 | 6 | 3 | 5 | 3 | 12 | 12 | 11 | 10 |
| BSTpr | 3 | 3 | 5 | 3 | 3 | 3 | 3 | 6 | 6 | 7 | 7 |
| BSTif | 3 | 3 | 9 | 5 | 5 | 5 | 3 | 7 | 3 | 7 | 7 |
| BSTtr | 3 | 3 | 9 | 3 | 3 | 3 | 3 | 9 | 7 | 7 | 6 |
| BSTd | 3 | 3 | 11 | 3 | 3 | 3 | 3 | 11 | 11 | 9 | 9 |
| BSTse | 1 | 1 | 1 | 1 | 1 | 1 | 1 | 1 | 1 | 1 | 1 |
| BAC | 1 | 1 | 1 | 1 | 1 | 1 | 1 | 1 | 1 | 1 | 1 |
| OT | 3 | 3 | 11 | 3 | 3 | 3 | 3 | 3 | 3 | 3 | 3 |
| ACB | 6 | 8 | 11 | 3 | 3 | 3 | 3 | 3 | 3 | 3 | 3 |
| LSr.m.v | 3 | 3 | 6 | 3 | 6 | 4 | 3 | 3 | 3 | 5 | 3 |
| LSr.m.d | 3 | 3 | 6 | 3 | 6 | 9 | 3 | 3 | 3 | 3 | 3 |
| LSr.vl | 3 | 3 | 6 | 3 | 3 | 3 | 3 | 7 | 3 | 5 | 5 |
| LSr.dl | 3 | 3 | 6 | 3 | 4 | 9 | 5 | 3 | 3 | 3 | 3 |
| LSc.v | 3 | 3 | 6 | 3 | 7 | 10 | 6 | 5 | 3 | 3 | 3 |
| LSc.d | 3 | 3 | 6 | 5 | 6 | 11 | 3 | 3 | 3 | 3 | 3 |
| LSv | 3 | 3 | 5 | 3 | 3 | 6 | 5 | 6 | 3 | 5 | 5 |
| SH | 3 | 3 | 7 | 7 | 5 | 12 | 3 | 3 | 3 | 3 | 3 |
| SF | 3 | 3 | 5 | 3 | 11 | 11 | 6 | 3 | 3 | 3 | 3 |
| FS | 3 | 11 | 11 | 6 | 3 | 3 | 3 | 9 | 9 | 6 | 6 |
| CP | 9 | 11 | 3 | 3 | 3 | 3 | 3 | 3 | 3 | 3 | 3 |
| AAA | 3 | 8 | 8 | 8 | 5 | 8 | 1 | 9 | 5 | 6 | 3 |
| CEAm | 1 | 1 | 7 | 3 | 3 | 3 | 1 | 10 | 11 | 6 | 7 |
| CEAl | 3 | 3 | 7 | 3 | 3 | 3 | 3 | 7 | 11 | 3 | 6 |
| CEAc | 1 | 1 | 7 | 1 | 3 | 5 | 1 | 9 | 9 | 7 | 9 |
| IA | 3 | 3 | 8 | 3 | 8 | 8 | 1 | 3 | 3 | 3 | 3 |
| MEAav | 3 | 3 | 7 | 3 | 3 | 5 | 3 | 7 | 3 | 6 | 2 |
| MEAad | 3 | 3 | 9 | 5 | 5 | 9 | 3 | 11 | 3 | 9 | 7 |
| MEApv | 3 | 3 | 7 | 5 | 3 | 5 | 3 | 10 | 3 | 9 | 9 |
| MEApd | 3 | 3 | 6 | 3 | 3 | 3 | 3 | 6 | 3 | 6 | 5 |
| BA | 1 | 1 | 1 | 1 | 1 | 1 | 1 | 1 | 1 | 1 | 1 |

TABLE 7B
Data matrices for the association and commissural connections of the rat cerebral nuclei
derived from collation of connection reports from the primary literature (continued)

| | BST | BST | BST | BST | BST | BST | BST | BST | BST | BST | |
|---|---|---|---|---|---|---|---|---|---|---|---|
| | dm | al | ov | ju | rh | pr | If | tr | d | se | BAC |
| GPm | 3 | 3 | 3 | 3 | 3 | 3 | 3 | 3 | 3 | 3 | 3 |
| GPl | 3 | 3 | 3 | 3 | 3 | 3 | 3 | 3 | 3 | 3 | 3 |
| SI | 5 | 7 | 1 | 1 | 7 | 6 | 1 | 1 | 5 | 1 | 1 |
| MA | 1 | 1 | 1 | 1 | 1 | 1 | 1 | 1 | 1 | 1 | 1 |
| MS | 4 | 3 | 3 | 3 | 3 | 3 | 3 | 3 | 3 | 3 | 3 |
| NDB | 3 | 7 | 3 | 3 | 3 | 3 | 3 | 3 | 3 | 3 | 3 |
| TRS | 3 | 3 | 3 | 3 | 3 | 3 | 3 | 3 | 3 | 3 | 3 |
| BSTam | 9 | 11 | 4 | 3 | 9 | 6 | 9 | 9 | 3 | 4 | 3 |
| BSTfu | 9 | 10 | 9 | 4 | 9 | 4 | 4 | 4 | 3 | 4 | 3 |
| BSTv | 10 | 7 | 5 | 3 | 7 | 3 | 3 | 5 | 3 | 3 | 3 |
| BSTmg | 11 | 7 | 6 | 3 | 11 | 5 | 5 | 3 | 3 | 3 | 3 |
| BSTdm | 0 | 10 | 6 | 3 | 9 | 6 | 9 | 7 | 5 | 3 | 3 |
| BSTal | 7 | 0 | 9 | 9 | 11 | 5 | 6 | 6 | 3 | 3 | 3 |
| BSTov | 6 | 9 | 0 | 9 | 9 | 3 | 7 | 7 | 3 | 3 | 3 |
| BSTju | 5 | 7 | 5 | 0 | 5 | 3 | 4 | 4 | 3 | 11 | 3 |
| BSTrh | 9 | 12 | 11 | 7 | 0 | 3 | 9 | 9 | 3 | 3 | 3 |
| BSTpr | 6 | 4 | 3 | 3 | 3 | 0 | 7 | 6 | 6 | 3 | 3 |
| BSTif | 5 | 5 | 3 | 3 | 3 | 6 | 0 | 9 | 6 | 5 | 3 |
| BSTtr | 6 | 6 | 6 | 3 | 7 | 6 | 9 | 0 | 5 | 3 | 3 |
| BSTd | 9 | 11 | 6 | 6 | 6 | 6 | 12 | 12 | 0 | 11 | 3 |
| BSTse | 1 | 1 | 1 | 1 | 1 | 1 | 1 | 1 | 1 | 0 | 1 |
| BAC | 1 | 1 | 1 | 1 | 1 | 1 | 1 | 1 | 1 | 1 | 0 |
| OT | 3 | 3 | 3 | 3 | 3 | 3 | 3 | 3 | 3 | 3 | 3 |
| ACB | 3 | 3 | 3 | 3 | 3 | 3 | 3 | 3 | 3 | 3 | 3 |
| LSr.m.v | 3 | 3 | 3 | 3 | 3 | 3 | 5 | 3 | 3 | 3 | 3 |
| LSr.m.d | 3 | 5 | 3 | 3 | 3 | 3 | 3 | 3 | 3 | 3 | 3 |
| LSr.vl | 5 | 6 | 3 | 3 | 3 | 6 | 3 | 3 | 3 | 3 | 6 |
| LSr.dl | 3 | 3 | 3 | 3 | 3 | 3 | 3 | 3 | 3 | 3 | 3 |
| LSc.v | 3 | 3 | 3 | 3 | 3 | 3 | 3 | 3 | 3 | 3 | 3 |
| LSc.d | 3 | 3 | 3 | 3 | 3 | 3 | 3 | 3 | 3 | 3 | 3 |
| LSv | 5 | 3 | 3 | 3 | 3 | 6 | 5 | 5 | 7 | 3 | 3 |
| SH | 3 | 5 | 3 | 3 | 3 | 3 | 3 | 3 | 3 | 3 | 3 |
| SF | 3 | 3 | 3 | 3 | 3 | 3 | 3 | 3 | 3 | 3 | 3 |
| FS | 6 | 7 | 3 | 3 | 6 | 6 | 3 | 3 | 3 | 3 | 3 |
| CP | 3 | 3 | 3 | 3 | 3 | 3 | 3 | 3 | 3 | 3 | 3 |
| AAA | 5 | 9 | 6 | 3 | 11 | 6 | 7 | 11 | 3 | 3 | 3 |
| CEAm | 6 | 11 | 11 | 5 | 12 | 5 | 7 | 10 | 6 | 3 | 3 |
| CEAl | 5 | 9 | 10 | 3 | 6 | 3 | 5 | 6 | 3 | 3 | 3 |
| CEAc | 6 | 12 | 6 | 5 | 11 | 5 | 6 | 7 | 6 | 3 | 3 |
| IA | 3 | 3 | 3 | 3 | 3 | 3 | 3 | 3 | 3 | 3 | 3 |
| MEAav | 2 | 9 | 3 | 3 | 2 | 6 | 7 | 7 | 2 | 11 | 3 |
| MEAad | 9 | 7 | 5 | 3 | 9 | 6 | 11 | 11 | 6 | 6 | 3 |
| MEApv | 6 | 6 | 5 | 3 | 3 | 7 | 11 | 12 | 6 | 3 | 3 |
| MEApd | 5 | 5 | 3 | 3 | 3 | 12 | 9 | 7 | 6 | 3 | 3 |
| BA | 1 | 1 | 1 | 1 | 1 | 1 | 1 | 1 | 1 | 1 | 1 |

TABLE 7C
Data matrices for the association and commissural connections of the rat cerebral nuclei
derived from collation of connection reports from the primary literature (continued)

| | OT | ACB | LSr.m.v | LSr.m.d | LSr.vl | LSr.dl | LSc.v | LSc.d | LSv | SH | SF |
|---|---|---|---|---|---|---|---|---|---|---|---|
| GPm | 3 | 3 | 3 | 3 | 3 | 3 | 3 | 3 | 3 | 3 | 3 |
| GPl | 3 | 3 | 3 | 3 | 3 | 3 | 3 | 3 | 3 | 3 | 3 |
| SI | 2 | 11 | 7 | 9 | 3 | 3 | 3 | 3 | 6 | 3 | 5 |
| MA | 3 | 3 | 3 | 3 | 3 | 3 | 3 | 3 | 3 | 3 | 3 |
| MS | 4 | 4 | 4 | 4 | 4 | 4 | 1 | 4 | 4 | 4 | 4 |
| NDB | 6 | 3 | 3 | 7 | 3 | 7 | 7 | 5 | 3 | 4 | 4 |
| TRS | 3 | 3 | 3 | 3 | 3 | 3 | 3 | 3 | 3 | 3 | 3 |
| BSTam | 7 | 12 | 7 | 3 | 7 | 6 | 6 | 5 | 7 | 3 | 5 |
| BSTfu | 3 | 9 | 6 | 3 | 6 | 5 | 3 | 5 | 6 | 3 | 5 |
| BSTv | 3 | 7 | 6 | 5 | 5 | 5 | 5 | 5 | 5 | 3 | 5 |
| BSTmg | 3 | 7 | 5 | 3 | 5 | 5 | 3 | 3 | 3 | 3 | 5 |
| BSTdm | 5 | 7 | 7 | 5 | 6 | 7 | 6 | 5 | 5 | 3 | 3 |
| BSTal | 5 | 6 | 6 | 5 | 6 | 6 | 5 | 5 | 3 | 3 | 3 |
| BSTov | 3 | 7 | 3 | 3 | 3 | 3 | 3 | 3 | 3 | 3 | 3 |
| BSTju | 3 | 6 | 3 | 3 | 3 | 5 | 3 | 3 | 3 | 3 | 5 |
| BSTrh | 7 | 10 | 6 | 3 | 6 | 6 | 5 | 5 | 6 | 3 | 5 |
| BSTpr | 3 | 5 | 5 | 3 | 7 | 6 | 6 | 3 | 7 | 3 | 3 |
| BSTif | 6 | 5 | 7 | 6 | 7 | 7 | 6 | 3 | 6 | 3 | 5 |
| BSTtr | 5 | 6 | 7 | 5 | 7 | 6 | 6 | 3 | 5 | 3 | 3 |
| BSTd | 9 | 9 | 3 | 3 | 10 | 3 | 3 | 3 | 11 | 3 | 3 |
| BSTse | 1 | 1 | 1 | 1 | 1 | 1 | 1 | 1 | 1 | 1 | 1 |
| BAC | 1 | 1 | 1 | 1 | 1 | 1 | 1 | 1 | 1 | 1 | 1 |
| OT | 0 | 3 | 3 | 3 | 3 | 3 | 3 | 3 | 3 | 3 | 3 |
| ACB | 3 | 0 | 3 | 3 | 3 | 3 | 3 | 3 | 3 | 3 | 3 |
| LSr.m.v | 3 | 3 | 0 | 5 | 5 | 3 | 3 | 3 | 3 | 3 | 9 |
| LSr.m.d | 3 | 3 | 7 | 0 | 3 | 3 | 5 | 7 | 3 | 3 | 6 |
| LSr.vl | 3 | 9 | 9 | 3 | 0 | 6 | 6 | 3 | 6 | 3 | 6 |
| LSr.dl | 3 | 3 | 7 | 6 | 6 | 0 | 7 | 6 | 3 | 3 | 7 |
| LSc.v | 3 | 3 | 9 | 3 | 3 | 3 | 0 | 5 | 3 | 3 | 7 |
| LSc.d | 3 | 3 | 9 | 9 | 3 | 9 | 3 | 0 | 3 | 3 | 3 |
| LSv | 3 | 3 | 5 | 3 | 6 | 3 | 6 | 3 | 0 | 3 | 5 |
| SH | 3 | 3 | 3 | 7 | 7 | 3 | 3 | 9 | 3 | 0 | 3 |
| SF | 3 | 3 | 6 | 7 | 3 | 6 | 3 | 3 | 3 | 3 | 0 |
| FS | 6 | 6 | 3 | 3 | 3 | 5 | 5 | 3 | 3 | 3 | 3 |
| CP | 3 | 3 | 3 | 3 | 3 | 3 | 3 | 3 | 3 | 3 | 3 |
| AAA | 8 | 8 | 5 | 3 | 3 | 3 | 3 | 3 | 3 | 3 | 3 |
| CEAm | 1 | 1 | 3 | 3 | 3 | 1 | 1 | 1 | 1 | 1 | 1 |
| CEAl | 3 | 3 | 3 | 3 | 3 | 3 | 3 | 3 | 3 | 3 | 3 |
| CEAc | 1 | 1 | 5 | 3 | 3 | 3 | 3 | 3 | 3 | 1 | 1 |
| IA | 8 | 8 | 3 | 3 | 3 | 3 | 3 | 3 | 3 | 3 | 3 |
| MEAav | 6 | 6 | 6 | 3 | 3 | 3 | 3 | 3 | 3 | 3 | 3 |
| MEAad | 6 | 6 | 9 | 5 | 10 | 6 | 6 | 6 | 7 | 3 | 5 |
| MEApv | 6 | 3 | 6 | 3 | 7 | 6 | 6 | 3 | 7 | 3 | 3 |
| MEApd | 3 | 3 | 5 | 3 | 7 | 6 | 6 | 3 | 7 | 3 | 5 |
| BA | 1 | 1 | 1 | 1 | 1 | 1 | 1 | 1 | 1 | 1 | 1 |

TABLE 7D
Data matrices for the association and commissural connections of the rat cerebral nuclei
derived from collation of connection reports from the primary literature (continued)

| | FS | CP | AAA | CEAm | CEAl | CEAc | IA | MEAav | MEAad | MEApv | MEApd | BA |
|---|---|---|---|---|---|---|---|---|---|---|---|---|
| GPm | 3 | 9 | 3 | 3 | 3 | 3 | 3 | 3 | 3 | 3 | 3 | 3 |
| GPl | 3 | 9 | 3 | 3 | 4 | 3 | 3 | 3 | 3 | 3 | 3 | 3 |
| SI | 7 | 6 | 6 | 9 | 6 | 5 | 6 | 6 | 9 | 7 | 9 | 5 |
| MA | 3 | 3 | 7 | 6 | 3 | 3 | 3 | 3 | 6 | 6 | 7 | 1 |
| MS | 3 | 3 | 3 | 3 | 3 | 6 | 3 | 3 | 3 | 3 | 7 | 3 |
| NDB | 3 | 3 | 3 | 3 | 6 | 7 | 3 | 7 | 7 | 7 | 7 | 3 |
| TRS | 3 | 3 | 3 | 3 | 3 | 3 | 3 | 3 | 3 | 3 | 3 | 3 |
| BSTam | 6 | 3 | 5 | 9 | 6 | 6 | 3 | 3 | 6 | 5 | 5 | 3 |
| BSTfu | 5 | 3 | 3 | 11 | 5 | 5 | 5 | 3 | 3 | 3 | 3 | 3 |
| BSTv | 3 | 3 | 3 | 9 | 5 | 7 | 5 | 3 | 6 | 3 | 3 | 3 |
| BSTmg | 5 | 3 | 5 | 9 | 3 | 6 | 3 | 3 | 3 | 3 | 3 | 3 |
| BSTdm | 6 | 3 | 6 | 10 | 5 | 6 | 3 | 3 | 7 | 6 | 6 | 3 |
| BSTal | 6 | 3 | 6 | 12 | 6 | 6 | 6 | 3 | 6 | 3 | 6 | 3 |
| BSTov | 5 | 5 | 3 | 10 | 7 | 6 | 5 | 3 | 3 | 3 | 3 | 3 |
| BSTju | 5 | 7 | 5 | 9 | 6 | 6 | 3 | 3 | 5 | 3 | 3 | 3 |
| BSTrh | 9 | 5 | 6 | 12 | 9 | 12 | 7 | 5 | 6 | 5 | 5 | 5 |
| BSTpr | 3 | 3 | 5 | 5 | 3 | 5 | 5 | 3 | 6 | 5 | 11 | 3 |
| BSTif | 3 | 3 | 6 | 6 | 3 | 6 | 5 | 6 | 9 | 9 | 7 | 3 |
| BSTtr | 6 | 3 | 9 | 10 | 3 | 7 | 7 | 6 | 7 | 6 | 6 | 3 |
| BSTd | 3 | 3 | 3 | 8 | 2 | 2 | 2 | 3 | 11 | 6 | 11 | 3 |
| BSTse | 1 | 1 | 1 | 1 | 1 | 1 | 1 | 1 | 1 | 1 | 1 | 1 |
| BAC | 1 | 1 | 1 | 1 | 1 | 1 | 1 | 1 | 1 | 1 | 1 | 1 |
| OT | 3 | 3 | 3 | 3 | 3 | 3 | 3 | 3 | 3 | 3 | 3 | 3 |
| ACB | 3 | 7 | 3 | 5 | 3 | 3 | 3 | 3 | 3 | 3 | 3 | 3 |
| LSr.m.v | 3 | 3 | 3 | 3 | 3 | 3 | 3 | 3 | 3 | 3 | 3 | 3 |
| LSr.m.d | 3 | 3 | 3 | 3 | 3 | 3 | 3 | 3 | 3 | 3 | 3 | 3 |
| LSr.vl | 6 | 3 | 3 | 3 | 3 | 3 | 3 | 3 | 3 | 3 | 3 | 3 |
| LSr.dl | 3 | 3 | 3 | 3 | 3 | 3 | 3 | 3 | 6 | 3 | 3 | 3 |
| LSc.v | 3 | 3 | 3 | 3 | 3 | 3 | 3 | 3 | 3 | 3 | 3 | 3 |
| LSc.d | 3 | 3 | 3 | 3 | 3 | 3 | 3 | 3 | 5 | 3 | 3 | 3 |
| LSv | 3 | 3 | 3 | 3 | 3 | 3 | 3 | 3 | 3 | 3 | 6 | 3 |
| SH | 9 | 3 | 6 | 6 | 3 | 3 | 3 | 3 | 3 | 3 | 3 | 3 |
| SF | 3 | 3 | 3 | 3 | 3 | 3 | 3 | 3 | 3 | 3 | 3 | 3 |
| FS | 0 | 6 | 6 | 11 | 5 | 9 | 6 | 3 | 6 | 3 | 5 | 3 |
| CP | 3 | 0 | 3 | 3 | 3 | 3 | 3 | 3 | 3 | 3 | 3 | 3 |
| AAA | 11 | 3 | 0 | 6 | 6 | 6 | 1 | 1 | 1 | 1 | 1 | 1 |
| CEAm | 6 | 3 | 5 | 0 | 5 | 6 | 3 | 3 | 3 | 3 | 3 | 3 |
| CEAl | 3 | 3 | 3 | 9 | 0 | 6 | 3 | 3 | 3 | 3 | 3 | 3 |
| CEAc | 7 | 3 | 5 | 6 | 9 | 0 | 3 | 3 | 3 | 3 | 3 | 3 |
| IA | 8 | 1 | 1 | 3 | 3 | 3 | 0 | 1 | 1 | 1 | 1 | 1 |
| MEAav | 6 | 3 | 4 | 6 | 6 | 7 | 5 | 0 | 7 | 7 | 7 | 3 |
| MEAad | 7 | 3 | 12 | 9 | 5 | 11 | 11 | 11 | 0 | 11 | 9 | 12 |
| MEApv | 6 | 3 | 7 | 6 | 3 | 7 | 7 | 12 | 11 | 0 | 9 | 12 |
| MEApd | 3 | 3 | 6 | 6 | 3 | 6 | 5 | 9 | 9 | 9 | 0 | 7 |
| BA | 1 | 1 | 1 | 1 | 1 | 1 | 1 | 1 | 1 | 1 | 1 | 0 |

TABLE 8A
Data matrices for the association and commissural connections of the rat cerebral nuclei
derived from collation of connection reports from the primary literature

| | GPm | GPl | SI | MA | MS | NDB | TRS | BSTam | BSTfu | BSTv | BSTmg |
|---|---|---|---|---|---|---|---|---|---|---|---|
| GPm | 3 | 3 | 3 | 3 | 3 | 3 | 3 | 3 | 3 | 3 | 3 |
| GPl | 3 | 3 | 3 | 3 | 3 | 3 | 3 | 3 | 3 | 3 | 3 |
| SI | 3 | 3 | 6 | 3 | 3 | 3 | 3 | 3 | 1 | 3 | 3 |
| MA | 3 | 3 | 3 | 6 | 3 | 3 | 3 | 3 | 3 | 3 | 3 |
| MS | 3 | 3 | 3 | 3 | 4 | 3 | 1 | 3 | 3 | 3 | 3 |
| NDB | 3 | 3 | 3 | 3 | 5 | 3 | 3 | 3 | 3 | 3 | 3 |
| TRS | 3 | 3 | 3 | 3 | 3 | 3 | 2 | 3 | 3 | 3 | 3 |
| BSTam | 3 | 3 | 5 | 3 | 3 | 5 | 3 | 6 | 5 | 3 | 3 |
| BSTfu | 3 | 3 | 5 | 3 | 3 | 3 | 3 | 6 | 5 | 3 | 3 |
| BSTv | 3 | 3 | 3 | 3 | 3 | 3 | 3 | 5 | 5 | 5 | 3 |
| BSTmg | 3 | 3 | 3 | 3 | 3 | 3 | 3 | 6 | 3 | 3 | 3 |
| BSTdm | 3 | 3 | 6 | 3 | 3 | 3 | 3 | 6 | 3 | 5 | 5 |
| BSTal | 3 | 3 | 5 | 3 | 3 | 3 | 3 | 5 | 6 | 5 | 5 |
| BSTov | 3 | 3 | 3 | 3 | 3 | 3 | 3 | 3 | 3 | 3 | 3 |
| BSTju | 3 | 3 | 3 | 3 | 3 | 3 | 3 | 3 | 3 | 3 | 3 |
| BSTrh | 3 | 3 | 6 | 3 | 3 | 3 | 3 | 6 | 5 | 3 | 3 |
| BSTpr | 3 | 3 | 3 | 3 | 3 | 3 | 3 | 3 | 3 | 5 | 3 |
| BSTif | 3 | 3 | 3 | 3 | 3 | 3 | 3 | 5 | 3 | 3 | 5 |
| BSTtr | 3 | 3 | 3 | 3 | 3 | 3 | 3 | 5 | 3 | 3 | 3 |
| BSTd | 3 | 3 | 3 | 3 | 3 | 3 | 3 | 3 | 3 | 3 | 3 |
| BSTse | 1 | 1 | 1 | 1 | 1 | 1 | 1 | 1 | 1 | 1 | 1 |
| BAC | 1 | 1 | 1 | 1 | 1 | 1 | 1 | 1 | 1 | 1 | 1 |
| OT | 1 | 1 | 1 | 1 | 1 | 1 | 1 | 1 | 1 | 1 | 1 |
| ACB | 3 | 3 | 3 | 3 | 3 | 3 | 3 | 3 | 3 | 3 | 3 |
| LSr.m.v | 3 | 3 | 3 | 3 | 3 | 3 | 3 | 3 | 3 | 3 | 3 |
| LSr.m.d | 3 | 3 | 3 | 3 | 3 | 3 | 3 | 3 | 3 | 3 | 3 |
| LSr.vl | 3 | 3 | 3 | 3 | 3 | 3 | 3 | 3 | 3 | 3 | 3 |
| LSr.dl | 3 | 3 | 3 | 3 | 3 | 5 | 3 | 3 | 3 | 3 | 3 |
| LSc.v | 3 | 3 | 3 | 3 | 3 | 3 | 5 | 3 | 3 | 3 | 3 |
| LSc.d | 3 | 3 | 3 | 3 | 3 | 3 | 3 | 3 | 3 | 3 | 3 |
| LSv | 3 | 3 | 3 | 3 | 3 | 3 | 3 | 3 | 3 | 3 | 3 |
| SH | 3 | 3 | 3 | 3 | 3 | 3 | 3 | 3 | 3 | 3 | 3 |
| SF | 3 | 3 | 3 | 3 | 3 | 3 | 3 | 3 | 3 | 3 | 3 |
| FS | 3 | 3 | 3 | 3 | 3 | 3 | 3 | 3 | 3 | 3 | 3 |
| CP | 3 | 3 | 3 | 3 | 3 | 3 | 3 | 3 | 3 | 3 | 3 |
| AAA | 3 | 3 | 3 | 3 | 3 | 3 | 1 | 3 | 3 | 3 | 3 |
| CEAm | 1 | 1 | 3 | 3 | 3 | 3 | 1 | 3 | 3 | 3 | 3 |
| CEAl | 3 | 3 | 3 | 3 | 3 | 3 | 3 | 3 | 3 | 3 | 3 |
| CEAc | 1 | 1 | 3 | 1 | 3 | 3 | 1 | 3 | 3 | 3 | 3 |
| IA | 3 | 3 | 3 | 3 | 3 | 3 | 1 | 3 | 3 | 3 | 3 |
| MEAav | 3 | 3 | 3 | 3 | 3 | 3 | 3 | 3 | 3 | 3 | 2 |
| MEAad | 3 | 3 | 3 | 3 | 3 | 3 | 3 | 3 | 3 | 3 | 3 |
| MEApv | 3 | 3 | 3 | 3 | 3 | 3 | 3 | 3 | 3 | 3 | 3 |
| MEApd | 3 | 3 | 3 | 3 | 3 | 3 | 3 | 3 | 3 | 3 | 3 |
| BA | 1 | 1 | 1 | 1 | 1 | 1 | 1 | 1 | 1 | 1 | 1 |

TABLE 8B
Data matrices for the association and commissural connections of the rat cerebral
nuclei derived from collation of connection reports from the primary literature (continued)

| | BSTdm | BSTal | BSTov | BSTju | BSTrh | BSTpr | BSTIf | BSTtr | BSTd | BSTse | BAC |
|---|---|---|---|---|---|---|---|---|---|---|---|
| GPm | 3 | 3 | 3 | 3 | 3 | 3 | 3 | 3 | 3 | 3 | 3 |
| GPl | 3 | 3 | 3 | 3 | 3 | 3 | 3 | 3 | 3 | 3 | 3 |
| SI | 3 | 3 | 1 | 1 | 3 | 3 | 1 | 1 | 3 | 1 | 1 |
| MA | 3 | 3 | 3 | 3 | 3 | 3 | 3 | 3 | 3 | 3 | 3 |
| MS | 3 | 3 | 3 | 3 | 3 | 3 | 3 | 3 | 3 | 3 | 3 |
| NDB | 3 | 3 | 3 | 3 | 3 | 3 | 3 | 3 | 3 | 3 | 3 |
| TRS | 3 | 3 | 3 | 3 | 3 | 3 | 3 | 3 | 3 | 3 | 3 |
| BSTam | 5 | 5 | 3 | 3 | 3 | 5 | 3 | 3 | 3 | 3 | 3 |
| BSTfu | 5 | 5 | 5 | 3 | 3 | 3 | 3 | 3 | 3 | 3 | 3 |
| BSTv | 5 | 3 | 3 | 3 | 3 | 3 | 3 | 3 | 3 | 3 | 3 |
| BSTmg | 5 | 3 | 3 | 3 | 5 | 3 | 3 | 3 | 3 | 3 | 3 |
| BSTdm | 5 | 5 | 3 | 3 | 3 | 5 | 6 | 5 | 5 | 3 | 3 |
| BSTal | 5 | 6 | 3 | 3 | 5 | 3 | 3 | 5 | 3 | 3 | 3 |
| BSTov | 3 | 3 | 3 | 3 | 3 | 3 | 3 | 3 | 3 | 3 | 3 |
| BSTju | 3 | 3 | 3 | 3 | 3 | 3 | 3 | 3 | 3 | 3 | 3 |
| BSTrh | 4 | 5 | 5 | 3 | 5 | 3 | 4 | 3 | 3 | 3 | 3 |
| BSTpr | 3 | 3 | 3 | 3 | 3 | 3 | 3 | 3 | 3 | 3 | 3 |
| BSTif | 3 | 3 | 3 | 3 | 3 | 3 | 5 | 3 | 3 | 3 | 3 |
| BSTtr | 3 | 3 | 3 | 3 | 3 | 3 | 3 | 3 | 3 | 3 | 3 |
| BSTd | 3 | 3 | 3 | 3 | 3 | 3 | 3 | 3 | 3 | 3 | 3 |
| BSTse | 1 | 1 | 1 | 1 | 1 | 1 | 1 | 1 | 1 | 1 | 1 |
| BAC | 1 | 1 | 1 | 1 | 1 | 1 | 1 | 1 | 1 | 1 | 1 |
| OT | 1 | 1 | 1 | 1 | 1 | 1 | 1 | 1 | 1 | 1 | 1 |
| ACB | 3 | 3 | 3 | 3 | 3 | 3 | 3 | 3 | 3 | 3 | 3 |
| LSr.m.v | 3 | 3 | 3 | 3 | 3 | 3 | 3 | 3 | 3 | 3 | 3 |
| LSr.m.d | 3 | 3 | 3 | 3 | 3 | 3 | 3 | 3 | 3 | 3 | 3 |
| LSr.vl | 3 | 3 | 3 | 3 | 3 | 3 | 3 | 3 | 3 | 3 | 3 |
| LSr.dl | 3 | 3 | 3 | 3 | 3 | 3 | 3 | 3 | 3 | 3 | 3 |
| LSc.v | 3 | 3 | 3 | 3 | 3 | 3 | 3 | 3 | 3 | 3 | 3 |
| LSc.d | 3 | 3 | 3 | 3 | 3 | 3 | 3 | 3 | 3 | 3 | 3 |
| LSv | 3 | 3 | 3 | 3 | 3 | 3 | 3 | 3 | 3 | 3 | 3 |
| SH | 3 | 3 | 3 | 3 | 3 | 3 | 3 | 3 | 3 | 3 | 3 |
| SF | 3 | 3 | 3 | 3 | 3 | 3 | 3 | 3 | 3 | 3 | 3 |
| FS | 3 | 3 | 3 | 3 | 3 | 3 | 3 | 3 | 3 | 3 | 3 |
| CP | 3 | 3 | 3 | 3 | 3 | 3 | 3 | 3 | 3 | 3 | 3 |
| AAA | 3 | 3 | 3 | 3 | 3 | 3 | 3 | 3 | 3 | 3 | 3 |
| CEAm | 3 | 3 | 3 | 3 | 3 | 3 | 3 | 3 | 3 | 3 | 3 |
| CEAl | 3 | 3 | 3 | 3 | 3 | 3 | 3 | 3 | 3 | 3 | 3 |
| CEAc | 3 | 3 | 3 | 3 | 3 | 3 | 3 | 3 | 3 | 3 | 3 |
| IA | 3 | 3 | 3 | 3 | 3 | 3 | 3 | 3 | 3 | 3 | 3 |
| MEAav | 2 | 3 | 3 | 3 | 2 | 3 | 3 | 3 | 2 | 3 | 3 |
| MEAad | 3 | 3 | 3 | 3 | 3 | 3 | 3 | 3 | 3 | 3 | 3 |
| MEApv | 3 | 3 | 3 | 3 | 3 | 3 | 3 | 3 | 3 | 3 | 3 |
| MEApd | 3 | 3 | 3 | 3 | 3 | 3 | 3 | 3 | 3 | 3 | 3 |
| BA | 1 | 1 | 1 | 1 | 1 | 1 | 1 | 1 | 1 | 1 | 1 |

TABLE 8C
Data matrices for the association and commissural connections of the rat cerebral
nuclei derived from collation of connection reports from the primary literature (continued)

| | OT | ACB | LSr.m.v | LSr.m.d | LSr.vl | LSr.dl | LSc.v | LSc.d | LSv | SH | SF |
|---|---|---|---|---|---|---|---|---|---|---|---|
| GPm | 3 | 3 | 3 | 3 | 3 | 3 | 3 | 3 | 3 | 3 | 3 |
| GPl | 3 | 3 | 3 | 3 | 3 | 3 | 3 | 3 | 3 | 3 | 3 |
| SI | 2 | 3 | 3 | 3 | 3 | 3 | 3 | 3 | 3 | 3 | 3 |
| MA | 3 | 3 | 3 | 3 | 3 | 3 | 3 | 3 | 3 | 3 | 3 |
| MS | 3 | 3 | 4 | 4 | 1 | 1 | 1 | 4 | 1 | 3 | 3 |
| NDB | 3 | 3 | 3 | 7 | 3 | 7 | 6 | 3 | 3 | 3 | 1 |
| TRS | 3 | 3 | 3 | 3 | 3 | 3 | 3 | 3 | 3 | 3 | 3 |
| BSTam | 3 | 5 | 5 | 3 | 5 | 5 | 3 | 5 | 5 | 3 | 3 |
| BSTfu | 3 | 5 | 3 | 3 | 3 | 3 | 3 | 3 | 3 | 3 | 3 |
| BSTv | 3 | 3 | 5 | 5 | 5 | 3 | 3 | 3 | 3 | 3 | 3 |
| BSTmg | 3 | 3 | 3 | 3 | 5 | 3 | 3 | 3 | 3 | 3 | 3 |
| BSTdm | 3 | 3 | 5 | 3 | 3 | 3 | 3 | 3 | 3 | 3 | 3 |
| BSTal | 3 | 3 | 5 | 3 | 3 | 3 | 5 | 5 | 3 | 3 | 3 |
| BSTov | 3 | 3 | 3 | 3 | 3 | 3 | 3 | 3 | 3 | 3 | 3 |
| BSTju | 3 | 3 | 3 | 3 | 3 | 3 | 3 | 3 | 3 | 3 | 3 |
| BSTrh | 3 | 3 | 3 | 3 | 3 | 3 | 3 | 3 | 3 | 3 | 3 |
| BSTpr | 3 | 3 | 3 | 3 | 3 | 3 | 3 | 3 | 3 | 3 | 3 |
| BSTif | 3 | 3 | 3 | 3 | 3 | 6 | 3 | 3 | 3 | 3 | 3 |
| BSTtr | 3 | 3 | 5 | 5 | 3 | 3 | 3 | 3 | 3 | 3 | 3 |
| BSTd | 3 | 3 | 3 | 3 | 3 | 3 | 3 | 3 | 3 | 3 | 3 |
| BSTse | 1 | 1 | 1 | 1 | 1 | 1 | 1 | 1 | 1 | 1 | 1 |
| BAC | 1 | 1 | 1 | 1 | 1 | 1 | 1 | 1 | 1 | 1 | 1 |
| OT | 1 | 1 | 1 | 1 | 1 | 1 | 1 | 1 | 1 | 1 | 1 |
| ACB | 3 | 3 | 3 | 3 | 3 | 3 | 3 | 3 | 3 | 3 | 3 |
| LSr.m.v | 3 | 3 | 3 | 3 | 3 | 3 | 3 | 3 | 3 | 3 | 3 |
| LSr.m.d | 3 | 3 | 3 | 3 | 3 | 3 | 3 | 3 | 3 | 3 | 3 |
| LSr.vl | 3 | 3 | 5 | 3 | 5 | 3 | 3 | 3 | 3 | 3 | 5 |
| LSr.dl | 3 | 3 | 5 | 3 | 3 | 3 | 3 | 3 | 3 | 3 | 5 |
| LSc.v | 3 | 3 | 3 | 3 | 3 | 3 | 3 | 3 | 3 | 3 | 5 |
| LSc.d | 3 | 3 | 3 | 3 | 3 | 3 | 3 | 5 | 3 | 3 | 3 |
| LSv | 3 | 3 | 3 | 3 | 3 | 3 | 3 | 3 | 3 | 3 | 3 |
| SH | 3 | 3 | 3 | 3 | 3 | 3 | 3 | 3 | 3 | 3 | 3 |
| SF | 3 | 3 | 3 | 3 | 3 | 3 | 3 | 3 | 3 | 3 | 3 |
| FS | 3 | 3 | 3 | 3 | 3 | 3 | 3 | 3 | 3 | 3 | 3 |
| CP | 3 | 3 | 3 | 3 | 3 | 3 | 3 | 3 | 3 | 3 | 3 |
| AAA | 3 | 3 | 3 | 3 | 3 | 3 | 3 | 3 | 3 | 3 | 3 |
| CEAm | 1 | 1 | 3 | 3 | 3 | 1 | 1 | 1 | 1 | 1 | 1 |
| CEAl | 3 | 3 | 3 | 3 | 3 | 3 | 3 | 3 | 3 | 3 | 3 |
| CEAc | 1 | 1 | 3 | 3 | 3 | 3 | 3 | 3 | 3 | 1 | 1 |
| IA | 3 | 3 | 3 | 3 | 3 | 3 | 3 | 3 | 3 | 3 | 3 |
| MEAav | 3 | 3 | 3 | 3 | 3 | 3 | 3 | 3 | 3 | 3 | 3 |
| MEAad | 3 | 3 | 3 | 3 | 3 | 3 | 3 | 3 | 3 | 3 | 3 |
| MEApv | 3 | 3 | 3 | 3 | 3 | 3 | 3 | 3 | 3 | 3 | 3 |
| MEApd | 3 | 3 | 3 | 3 | 3 | 3 | 3 | 3 | 3 | 3 | 3 |
| BA | 1 | 1 | 1 | 1 | 1 | 1 | 1 | 1 | 1 | 1 | 1 |

TABLE 8D
Data matrices for the association and commissural connections of the rat cerebral
nuclei derived from collation of connection reports from the primary literature (continued)

| | FS | CP | AAA | CEAm | CEAl | CEAc | IA | MEAav | MEAad | MEApv | MEApd | BA |
|---|---|---|---|---|---|---|---|---|---|---|---|---|
| GPm | 3 | 3 | 3 | 3 | 3 | 3 | 3 | 3 | 3 | 3 | 3 | 3 |
| GPl | 3 | 3 | 3 | 3 | 3 | 3 | 3 | 3 | 3 | 3 | 3 | 3 |
| SI | 3 | 3 | 3 | 3 | 3 | 3 | 3 | 3 | 3 | 3 | 3 | 3 |
| MA | 3 | 3 | 3 | 3 | 3 | 3 | 3 | 3 | 3 | 3 | 3 | 3 |
| MS | 3 | 3 | 3 | 3 | 3 | 3 | 3 | 3 | 3 | 3 | 3 | 3 |
| NDB | 3 | 3 | 3 | 3 | 3 | 3 | 3 | 3 | 3 | 3 | 3 | 3 |
| TRS | 3 | 3 | 3 | 3 | 3 | 3 | 3 | 3 | 3 | 3 | 3 | 3 |
| BSTam | 3 | 3 | 3 | 5 | 3 | 3 | 3 | 3 | 3 | 3 | 3 | 3 |
| BSTfu | 3 | 3 | 3 | 6 | 3 | 3 | 3 | 3 | 3 | 3 | 3 | 3 |
| BSTv | 3 | 3 | 3 | 3 | 3 | 3 | 3 | 3 | 3 | 3 | 3 | 3 |
| BSTmg | 3 | 3 | 3 | 3 | 3 | 3 | 3 | 3 | 3 | 3 | 3 | 3 |
| BSTdm | 3 | 3 | 3 | 6 | 3 | 3 | 3 | 3 | 3 | 3 | 3 | 3 |
| BSTal | 3 | 3 | 3 | 3 | 3 | 3 | 3 | 3 | 3 | 3 | 3 | 3 |
| BSTov | 3 | 3 | 3 | 3 | 3 | 3 | 3 | 3 | 3 | 3 | 3 | 3 |
| BSTju | 3 | 3 | 3 | 3 | 3 | 3 | 3 | 3 | 3 | 3 | 3 | 3 |
| BSTrh | 3 | 3 | 3 | 6 | 5 | 5 | 3 | 3 | 3 | 3 | 3 | 3 |
| BSTpr | 3 | 3 | 3 | 3 | 3 | 3 | 3 | 3 | 3 | 3 | 3 | 3 |
| BSTif | 3 | 3 | 3 | 3 | 3 | 3 | 3 | 3 | 5 | 3 | 3 | 3 |
| BSTtr | 3 | 3 | 3 | 3 | 3 | 3 | 3 | 3 | 3 | 3 | 3 | 3 |
| BSTd | 3 | 3 | 3 | 3 | 2 | 2 | 2 | 3 | 3 | 3 | 3 | 3 |
| BSTse | 1 | 1 | 1 | 1 | 1 | 1 | 1 | 1 | 1 | 1 | 1 | 1 |
| BAC | 1 | 1 | 1 | 1 | 1 | 1 | 1 | 1 | 1 | 1 | 1 | 1 |
| OT | 1 | 1 | 1 | 1 | 1 | 1 | 1 | 1 | 1 | 1 | 1 | 1 |
| ACB | 3 | 3 | 3 | 3 | 3 | 3 | 3 | 3 | 3 | 3 | 3 | 3 |
| LSr.m.v | 3 | 3 | 3 | 3 | 3 | 3 | 3 | 3 | 3 | 3 | 3 | 3 |
| LSr.m.d | 3 | 3 | 3 | 3 | 3 | 3 | 3 | 3 | 3 | 3 | 3 | 3 |
| LSr.vl | 3 | 3 | 3 | 3 | 3 | 3 | 3 | 3 | 3 | 3 | 3 | 3 |
| LSr.dl | 3 | 3 | 3 | 3 | 3 | 3 | 3 | 3 | 3 | 3 | 3 | 3 |
| LSc.v | 3 | 3 | 3 | 3 | 3 | 3 | 3 | 3 | 3 | 3 | 3 | 3 |
| LSc.d | 3 | 3 | 3 | 3 | 3 | 3 | 3 | 3 | 3 | 3 | 3 | 3 |
| LSv | 3 | 3 | 3 | 3 | 3 | 3 | 3 | 3 | 3 | 3 | 3 | 3 |
| SH | 3 | 3 | 3 | 3 | 3 | 3 | 3 | 3 | 3 | 3 | 3 | 3 |
| SF | 3 | 3 | 3 | 3 | 3 | 3 | 3 | 3 | 3 | 3 | 3 | 3 |
| FS | 3 | 3 | 3 | 3 | 3 | 3 | 3 | 3 | 3 | 3 | 3 | 3 |
| CP | 3 | 3 | 3 | 3 | 3 | 3 | 3 | 3 | 3 | 3 | 3 | 3 |
| AAA | 3 | 3 | 3 | 3 | 3 | 3 | 1 | 1 | 1 | 1 | 1 | 1 |
| CEAm | 3 | 3 | 3 | 3 | 3 | 3 | 3 | 3 | 3 | 3 | 3 | 3 |
| CEAl | 3 | 3 | 3 | 3 | 3 | 3 | 3 | 3 | 3 | 3 | 3 | 3 |
| CEAc | 3 | 3 | 3 | 3 | 3 | 3 | 3 | 3 | 3 | 3 | 3 | 3 |
| IA | 3 | 1 | 1 | 3 | 3 | 3 | 3 | 1 | 1 | 1 | 1 | 1 |
| MEAav | 3 | 3 | 3 | 3 | 3 | 3 | 3 | 3 | 3 | 3 | 3 | 3 |
| MEAad | 3 | 3 | 3 | 3 | 3 | 3 | 3 | 3 | 3 | 3 | 3 | 3 |
| MEApv | 3 | 3 | 3 | 3 | 3 | 3 | 3 | 3 | 3 | 3 | 3 | 3 |
| MEApd | 3 | 3 | 3 | 3 | 3 | 3 | 3 | 3 | 3 | 3 | 3 | 3 |
| BA | 1 | 1 | 1 | 1 | 1 | 1 | 1 | 1 | 1 | 1 | 1 | 1 |

TABLE 9
Key for Tables 5-8

| Description | Raw Value | Binned Value |
|---|---|---|
| Same origin & termination= | 0 | 0 |
| No data= | 1 | 0 |
| Unclear= | 2 | 0 |
| Absent= | 3 | 0 |
| Axons of passage= | 4 | 2 |
| Very weak= | 5 | 1 |
| Weak= | 6 | 2 |
| Weak to moderate= | 7 | 3 |
| Present (value unreported)= | 8 | 4 |
| Moderate= | 9 | 4 |
| Moderate to strong= | 10 | 5 |
| Strong= | 11 | 6 |
| Very strong= | 12 | 7 |

Table 5A-5D: CN 1-1 binned. Table 6A-D: CN 1-2 binned. Cerebral nuclei (CN) association and commissural connection matrices with connection weights represented on an ordinal 0-7 scale—these data were used for modularity analysis. Table 7A-D: CN 1-1 raw. Table 8A-D: CN 1-2 raw. CN association and commissural connection matrices based on connection reported values and represented as weights on an ordinal 0-12 scale. Connection matrix directionality is from y axis to x axis. The key to Tables 5-8 is provided in Table 9.

### Example 3

### Further Elaboration of the Distribution of Agouti-Related Peptide-Immunoreactive Axons Using a Canonical Rat Brain Atlas in the Adult Male Rat: High Spatial Resolution Analysis of Rostral Forebrain Regions

Agouti-related peptide (AgRP) is a neuropeptide intensively studied for its role in feeding control; despite this, its brain expression is only partially determined. Here, an immunocytochemical investigation of AgRP chemoarchitecture in the rat forebrain is described, with additional rostral forebrain analysis of AGRP axon distribution. AgRP-immunoreactive (ir) axons were identified and mapped their distribution digitally to sequential levels of a canonical rat brain atlas (L. W. Swanson, Brain Maps, 2004). This was accomplished with referenced Nissl cytoarchitecture, the use of camera lucida drawings, and careful determination of plane of section (Zséli G et al., (2016) J Comp Neurol 524:2803). Fixed frozen brain sections of an adult male Sprague-Dawley rat were incubated with a rabbit polyclonal antibody raised against the 83-132 amino acid sequence of human AgRP (Phoenix). Labelling was visualized with 3,3′-diaminobenzidine, and the data were mapped with the aid of darkfield microscopy. AGRP-ir axon distribution was enumerated for semi-quantitative analysis with the use of Axiome C software.

The cerebral cortex displayed no AgRP-ir except for very sparse labeling in midline structures, notably the dorsal tenia tecta (TTd). In the striatum, there was low to moderate AgRP-ir in the nucleus accumbens (ACB) and the rostroventral part of the lateral septal nucleus (LSr); surrounding areas were devoid of axons. In the pallidum, there was sparse labelling in the substantia innominata (SI). In contrast, the bed nuclei of the stria terminalis (BST) had high expression of AgRP-ir axons, but low to moderate labeling in some BST subdivisions (oval, juxtacapsular). In the thalamus, the paraventricular thalamic- (PVT) and paratenial (PT) nuclei displayed a low (caudal) to high (rostral) AGRP-ir axon density. In the hypothalamus, dense AgRP-ir axons were observed in the paraventricular- (PVH), periventricular- (PV), arcuate- (ARH) and dorsomedial (DMH) hypothalamic nuclei. Moderately dense AgRP-ir was present in the anterior hypothalamic- (AHA), medial preoptic- (MPO), and lateral hypothalamic (LHA) areas. Sparse AgRP-ir was found in the anterior hypothalamic- (AHN) and ventromedial hypothalamic (VMH) nuclei, and the retrochiasmatic area (RCH).

Collectively, this data provide high spatial resolution rat brain atlas maps of AgRP-ir distribution and will aid in comparing other chemoarchitecture mapped to the same reference atlas. These data may also allow the precise targeting of interventions in forebrain regions that receive inputs from AgRP-expressing neurons.

### Example 4

A Comparative High Spatial Resolution Analysis of Genetic Markers for Neuronal GABA and Glutamate in the Rat Hypothalamus with Axiome C

Fast synaptic neurotransmission in the brain predominantly involves the amino acid neurotransmitters glutamate (GLU) and gamma-Aminobutyric acid (GABA). Both play a critical and pervasive role in normal brain function. Dysfunction of GABA and GLU neural circuits is implicated in numerous brain diseases. While much is known about the actions and brain expression of GLU and GABA in the cerebral cortex, cerebral nuclei, and thalamus, substantially less is known at the level of the hypothalamus. To increase understanding of GABAergic and glutamatergic hypothalamic neural circuits, a systematic high spatial resolution comparative analysis of genetic markers for both using in situ hybridization (ISH) is performed. For GABA, 35S-labeled riboprobes were used to detect the presence of mRNA for two isoforms of the GABA synthetic enzyme glutamate decarboxylase (GAD-65, GAD-67); for GLU a 35S-labeled riboprobe was used to detect a vesicular glutamate transporter (VGLUT2) mRNA, which shows abundant hypothalamic expression. Series of sequential brain sections were subjected to ISH for each riboprobe; an adjacent series was processed for Nissl cytoarchitecture.

Analysis of 83 cytoarchitecturally defined hypothalamic regions (following the rat brain atlas of Swanson, 2004) using Axiome C revealed the percentage of regions with detectable signal: GAD-65 (88%), GAD-67 (82%), VGLUT2 (94%). Using a 4-rank approach, regions with high or very high ranked signal included, for GAD-65: subfornical organ (SFO), lateral preoptic area (LPO), lateral hypothalamic area perifornical region, and the following hypothalamic nuclei: anterodorsal- and ventrolateral preoptic, suprachiasmatic (SCH), medial preoptic (MPN), anterior (AHN), arcuate (ARH), dorsomedial, periventricular posterior part (PVp), and tuberal; for GAD-67: SFO, SCH, AHN, ARH, PVp, tuberomammillary nucleus, and LHA posterior region; for VGLUT2: LHA anterior region, median preoptic- and anteroventral periventricular nuclei, SFO, MPN, AHN, and the following hypothalamic nuclei: supraoptic, ventromedial, posterior, medial mammillary, and subthalamic. Differences in inter- and intraregional reporter signal level and distribution were most apparent between VGLUT2 and the GAD isoforms; however, substantial differences were also noted between GAD-65 and GAD-67. These data inform a developing model of hypothalamic neural circuitry and support a continuing effort to obtain a comprehensive network model for the mammalian brain.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

1. A computer-implemented method for modeling connections of the central nervous system (CNS), the method comprising:
generating, with a processor, a plurality of connections corresponding to CNS data, wherein each of the plurality of connections comprises an origin, a termination, and a degree of connection;
storing the plurality of connections in a first memory location;
receiving a nervous system atlas and storing the nervous system atlas in a second memory location;
matching, with a processor on a connection-by-connection basis, each origin and each termination of the plurality of connections to a corresponding position of the nervous system atlas to produce an annotated connection matrix; and
converting the annotated connection matrix to one or more modules, wherein the one or modules comprise an aggregated ranking of connections exceeding a threshold level.

2. The method of claim 1, further comprising deriving the data from CNS connection information.

3. The method of claim 1, further comprising deriving the data from CNS gene expression data.

4. The method of claim 3, wherein the CNS gene expression data comprises expressions of a neurotransmitter, a neurotransmitter receptor, or a CNS cellular marker.

5. The method of claim 1, further comprising presenting the one or more modules as two-dimensional models.

6. The method of claim 1, further comprising projecting the one or more modules onto the nervous system atlas.

7. The method of claim 1, wherein the ranking of connections comprises one or more of: a node degree, node strength, node betweenness, and node closeness.

8. The method of claim 1, wherein converting the annotated connection matrix includes partitioning the annotated connection matrix into a plurality of modules by modularity maximization.

9. The method of claim 1, wherein the plurality of connections correspond to data of cerebral nuclei.

10. A non-transitory computer-readable medium with instructions stored thereon, that upon execution by a processor of a computing device, perform operations comprising:
generating, with the processor, a plurality of connections corresponding to CNS data, wherein each of the plurality of connections comprises an origin, a termination, and a degree of connection;
storing the plurality of connections in a first memory location;
receiving a nervous system atlas and storing the nervous system atlas in a second memory location;
matching, with the processor on a connection-by-connection basis, each origin and each termination of the plurality of connections to a corresponding position of the nervous system atlas to produce an annotated connection matrix; and
converting the annotated connection matrix to one or more modules, wherein the one or modules comprise an aggregated ranking of connections exceeding a threshold level.

11. The non-transitory computer-readable medium of claim 10, wherein the operations further comprise deriving the data from CNS connection information.

12. The non-transitory computer-readable medium of claim 10, wherein the operations further comprise deriving the data from CNS gene expression data.

13. The non-transitory computer-readable medium of claim 12, wherein the CNS gene expression data comprises expressions of a neurotransmitter, a neurotransmitter receptor, or a CNS cellular marker.

14. The non-transitory computer-readable medium of claim 10, wherein the plurality of connections correspond to data of cerebral nuclei.

15. The non-transitory computer-readable medium of claim 10, wherein the steps further comprise presenting the one or more modules as two-dimensional models.

16. The non-transitory computer-readable medium of claim 10, wherein the steps further comprise projecting the one or more modules onto the nervous system atlas.

17. The non-transitory computer-readable medium of claim 10, wherein the ranking of connections comprises one or more of: a node degree, node strength, node betweenness, and node closeness.

18. A system for modeling connections of the CNS, the system comprising:
a processor that generates a plurality of connections corresponding to CNS data, wherein each of the plurality of connections comprises an origin, a termination, and a degree of connection;
a first memory location for storing the plurality of connections;
a second memory location for storing a received a nervous system atlas and storing the nervous system atlas;
wherein the processor is further configured to:
match, on a connection-by-connection basis, each origin and each termination of the plurality of connections to a corresponding position of the nervous system atlas to produce an annotated connection matrix; and
convert the annotated connection matrix to one or more modules, wherein the one or modules comprise an aggregated ranking of connections exceeding a threshold level.

19. The system of claim 18, wherein the processor is further configured to derive the data from CNS connection information.

20. The system of claim 18, wherein the processor is configured to derive the data from CNS gene expression data.

21-24. (canceled)
